SEQUENCE LISTING (60 sequences)

SEQ ID NO: 1 - DNA, 56 bp, Escherichia coli
acaacacattttactgctgtgaactttgttgtaatcctgcctgtgctggatgttat

SEQ ID NO: 2 - protein, 19 aa, Escherichia coli
NNTFYCCELCCNPACAGCY

SEQ ID NO: 3 - protein, 18 aa, Escherichia coli
NTFYCCELCCYPACAGCN

SEQ ID NO: 4 - DNA, 56 bp, Escherichia coli
atagtagcaattactgctgtgaattgtgttgtaatcctgcttgtaacgggtgctat

SEQ ID NO: 5 - protein, 19 aa, Artificial Sequence
  Synthetic Sequence
NSSNYCCELCCNPACNGCY

SEQ ID NO: 6 - protein, 15 aa, Artificial Sequence
  Synthetic Sequence
PNTCEICAYAACTGC

SEQ ID NO: 7 - protein, 18 aa, Artificial Sequence
  Synthetic Sequence
NNTFYCCELCCNPACAGC

SEQ ID NO: 8 - protein, 17 aa, Artificial Sequence
  Synthetic Sequence
NTFYCCELCCNPACAGC

SEQ ID NO: 9 - protein, 16 aa, Artificial Sequence
  Synthetic Sequence
TFYCCELCCNPACAGC

SEQ ID NO: 10 - protein, 15 aa, Artificial Sequence
  Synthetic Sequence
FYCCELCCNPACAGC

SEQ ID NO: 11 - protein, 14 aa, Artificial Sequence
  Synthetic Sequence
YCCELCCNPACAGC

SEQ ID NO: 12 - protein, 13 aa, Artificial Sequence
  Synthetic Sequence
CCELCCNPACAGC

SEQ ID NO: 13 - protein, 18 aa, Artificial Sequence
  Synthetic Sequence
NTFYCCELCCNPACAGCY

SEQ ID NO: 14 - protein, 17 aa, Artificial Sequence
  Synthetic Sequence
TFYCCELCCNPACAGCY

SEQ ID NO: 15 - protein, 16 aa, Artificial Sequence
  Synthetic Sequence
FYCCELCCNPACAGCY

SEQ ID NO: 16 - protein, 15 aa, Artificial Sequence
  Synthetic Sequence
YCCELCCNPACAGCY

SEQ ID NO: 17 - protein, 14 aa, Artificial Sequence
  Synthetic Sequence
CCELCCNPACAGCY

SEQ ID NO: 18 - protein, 17 aa, Artificial Sequence
  Synthetic Sequence
NTFYCCELCCYPACAGC

SEQ ID NO: 19 - protein, 16 aa, Artificial Sequence
  Synthetic Sequence
TFYCCELCCYPACAGC

SEQ ID NO: 20 - protein, 15 aa, Artificial Sequence
  Synthetic Sequence
FYCCELCCYPACAGC

SEQ ID NO: 21 - protein, 14 aa, Artificial Sequence
  Synthetic Sequence
YCCELCCYPACAGC

SEQ ID NO: 22 - protein, 13 aa, Artificial Sequence
  Synthetic Sequence
CCELCCYPACAGC

SEQ ID NO: 23 - protein, 17 aa, Artificial Sequence
  Synthetic Sequence
TFYCCELCCYPACAGCN

SEQ ID NO: 24 - protein, 16 aa, Artificial Sequence
  Synthetic Sequence
FYCCELCCYPACAGCN

SEQ ID NO: 25 - protein, 15 aa, Artificial Sequence
  Synthetic Sequence
YCCELCCYPACAGCN

SEQ ID NO: 26 - protein, 14 aa, Artificial Sequence
  Synthetic Sequence
CCELCCYPACAGCN

SEQ ID NO: 27 - protein, 18 aa, Artificial Sequence
  Synthetic Sequence
NSSNYCCELCCNPACTGC

SEQ ID NO: 28 - protein, 17 aa, Artificial Sequence
  Synthetic Sequence
SSNYCCELCCNPACTGC

SEQ ID NO: 29 - protein, 16 aa, Artificial Sequence
  Synthetic Sequence
SNYCCELCCNPACTGC

SEQ ID NO: 30 - protein, 15 aa, Artificial Sequence
  Synthetic Sequence
NYCCELCCNPACTGC

SEQ ID NO: 31 - protein, 14 aa, Artificial Sequence
  Synthetic Sequence
YCCELCCNPACTGC

SEQ ID NO: 32 - protein, 13 aa, Artificial Sequence
  Synthetic Sequence
CCELCCNPACTGC

SEQ ID NO: 33 - protein, 18 aa, Artificial Sequence
  Synthetic Sequence
SSNYCCELCCNPACTGCY

SEQ ID NO: 34 - protein, 17 aa, Artificial Sequence
  Synthetic Sequence
SNYCCELCCNPACTGCY

SEQ ID NO: 35 - protein, 16 aa, Artificial Sequence
  Synthetic Sequence
NYCCELCCNPACTGCY

SEQ ID NO: 36 - protein, 15 aa, Artificial Sequence
  Synthetic Sequence
YCCELCCNPACTGCY

SEQ ID NO: 37 - protein, 14 aa, Artificial Sequence
  Synthetic Sequence
CCELCCNPACTGCY

SEQ ID NO: 38 - protein, 18 aa, Artificial Sequence
  Synthetic Sequence
NTFYCCELCCNPACAGCY

SEQ ID NO: 39 - protein, 18 aa, Artificial Sequence
  Synthetic Sequence
NTFYCCELCCAPACAGCY

SEQ ID NO: 40 - protein, 18 aa, Artificial Sequence
  Synthetic Sequence
NTFYCCELCCNAACAGCY

SEQ ID NO: 41 - protein, 17 aa, Artificial Sequence
  Synthetic Sequence
NTFYCCELCCNPACAGC

SEQ ID NO: 42 - protein, 15 aa, Artificial Sequence
  Synthetic Sequence
YCCELCCNPACAGCY

SEQ ID NO: 43 - protein, 14 aa, Artificial Sequence
  Synthetic Sequence
YCCELCCNPACAGC

SEQ ID NO: 44 - protein, 14 aa, Artificial Sequence
  Synthetic Sequence
CCELCCNPACAGCY

SEQ ID NO: 45 - protein, 13 aa, Artificial Sequence
  Synthetic Sequence
CCELCCNPACAGC

SEQ ID NO: 46 - protein, 25 aa, Yersinia enterocolitica
QACDPPSPPAEVCCDVCCNPACAGC

SEQ ID NO: 47 - protein, 16 aa, Vibrio cholerae
IDCCICCNPACFGCLN

SEQ ID NO: 48 - protein, 18 aa, Yersinia enterocolitica
SSDWDCCDVCCNPACAGC

SEQ ID NO: 49 - protein, 19 aa, Artificial Sequence
  Synthetic Sequence
NSSNYCCELCCYPACTGCY

SEQ ID NO: 50 - protein, 13 aa, Artificial Sequence
  Synthetic Sequence
CCDVCCNPACTGC

SEQ ID NO: 51 - protein, 14 aa, Artificial Sequence
  Synthetic Sequence
CCDVCCYPACTGCY

SEQ ID NO: 52 - protein, 14 aa, Artificial Sequence
  Synthetic Sequence
CCDLCCNPACAGCY

SEQ ID NO: 53 - protein, 14 aa, Artificial Sequence
  Synthetic Sequence
CCQLCCNPACTGCY

SEQ ID NO: 54 - protein, 15 aa, Homo sapiens
PGTCEICAYAACTGC

SEQ ID NO: 55 - protein, 15 aa, Didelphis virginiana
QEDCELCINVACTGC

SEQ ID NO: 56 - protein, 16 aa, Homo sapiens
NDDCELCVNVACTGCL

SEQ ID NO: 57 - protein, 94 aa, Homo sapiens
VTVQDGNFSFSLESVKKLKDLQEPQEPRVGKLRNFAPIPGEPVVPILCSNPNFPEELKPLCKEPNAQEILQRLEEIAEDPGTCEICAYAACTGC

SEQ ID NO: 58 - protein, 86 aa, Homo sapiens
VYIQYQGFRVQLESMKKLSDLEAQWAPSPRLQAQSLLPAVCHHPALPQDLQPVCASQEASSIFKTLRTIANDDCELCVNVACTGCL

SEQ ID NO: 59 - protein, 14 aa, Artificial Sequence
  Synthetic Sequence
CCEYCCNPACTGCY

SEQ ID NO: 60 - protein, 16 aa, Artificial Sequence
  Synthetic Sequence
NDECELCVNVACTGCL

(19) United States
(12) Patent Application Publication
(10) Pub. No.: US 2020/0376068 A1
(43) Pub. Date: Dec. 3, 2020

(54) PROTECTION OF NORMAL TISSUE IN CANCER TREATMENT

(71) Applicant: THOMAS JEFFERSON UNIVERSITY, Philadelphia, AP (US)

(72) Inventor: Scott A. Waldman, Philadelphia, PA (US)

(21) Appl. No.: 16/639,742

(22) PCT Filed: Aug. 10, 2018
(86) PCT No.: PCT/US18/46273

§ 371 Date: Feb. 17, 2020

Related U.S. Application Data
(60) Provisional application No. 62/547,560, filed on Aug. 18, 2017.

(51) Int. Cl. A61K 38/00; A61P 35/00

(52) U.S. Cl. CPC A61K 38/005; A61K 45/06; A61P 35/00

(57) ABSTRACT

Methods of treating individuals who have cancer are disclosed. In some methods, the cancers may lack functional guanylyl cyclase C and/or p53. In some methods, the methods comprise protecting gastrointestinal cells from genotoxic damage by administering one or more compounds sufficient to elevate intracellular cGMP in the gastrointestinal cells, and then administering chemotherapy and/or radiation therapy to kill cancer cells. In some methods, the method comprise administering one or more guanylyl cyclase C agonist compounds to intestinal stem cells in the individual an amount of sufficient to activate guanylyl cyclase C of the intestinal stem cells and elevate intracellular cGMP in the intestinal stem cells, and then administering chemotherapy and/or radiation therapy to kill cancer cells.

### FIELD OF THE INVENTION

The present invention relates to compositions for and methods of protecting an individual from serious and possibly lethal side effects associated with cancer chemotherapy and radiation therapy.

### BACKGROUND OF THE INVENTION

Cancer is a leading cause of death worldwide: it accounted for 7-8 million deaths (approximately 13% of all deaths) yearly since 2004. Deaths from cancer worldwide are projected to continue rising, with an estimated 12 million deaths in 2030. Lung, stomach, liver, colon and breast cancer cause the most cancer deaths each year. In US, cancer is the second cause of death in adults and causes above half a million deaths each year. Lung, prostate, breast and colon cancers are the leading causes of cancer related deaths.

Chemotherapy and radiation therapy, the two most common types of cancer treatment, work by destroying fast-growing cells such as cancer cells. Chemotherapy and radiation therapy are extremely toxic treatments because they kill rapidly dividing cells including normal, non-cancerous dividing cells. Therefore, as an unwanted side effect of chemotherapy and radiation, other types of fast-growing normal cells in the body, such as hematopoietic, hair and gastrointestinal tract (GI) cells, are also damaged and killed. Severe side effects of chemo- and radiation therapy discourage people from continuing their therapy, limit the efficacy of the treatments and sometimes even kill patients. The toxicity which is manifested by these side effects limits the dosages of chemotherapeutic and radiation a patient can be administered.

Gastrointestinal toxicities occur in clinical practice as a side effect of treatment with radiation and some chemotherapeutic agents. Additionally, a 1-3% treatment related death rate has been observed in this and many other large Phase III clinical trials. While side effects can be lethal, most acute side effects improve over time. Some chronic side effects of cancer treatment, however, can lead to lifelong morbidity. Minimizing the side effects of chemotherapy and radiation remains one of the top priorities for patients and doctors like.

Mice irradiated with >15 Gy of radiation die between 7 and 12 days after treatment from complications of damage to the small intestine—gastrointestinal (GI) syndrome—prior to development of lethal effect of hemopoietic cells. Massive p53-dependent apoptosis is observed following lethal doses of radiation, suggesting that p53 is a determinant of radiation-induced death. However, while the reaction of small intestine to gamma radiation has been well examined at a pathomorphological level, the exact cause of GI lethality has not been fully elucidated. Death may occur as a direct consequence of the damage of epithelial crypt cells and followed denudation of villi leading to fluid and electrolyte imbalance, bacteremia and endotoxemia. Besides inflammation and stromal responses, endothelial dysfunctions may also contribute to lethality.

Garin-Laflam, et al. Am. J. Physiol Gastrointest Liver Physiol 2009 296 G740-9, report the involvement of GCC and cGMP in the prevention of radiation induced intestinal epithelial apoptosis. These studies which relate relative number of intestinal cells undergoing apoptosis, not survival from GI syndrome, were conducted to resolve whether GCC activation has a pro-apoptotic effect, an anti-apoptotic effect or neither in a model of apoptosis involving cells that express GCC. In these studies, intestinal tissue was removed from mice and the number of cells in the resected tissue undergoing apoptosis was measured. Tissue was obtained from various wild type and genetically modified mice as well as mice injected with a cGMP analog. The experiments showed that tissue removed from irradiated mice included a larger number of cells undergoing apoptosis compared to levels observed in tissue from non-irradiated animals. Further, the data show tissue removed from irradiated mice that lacked genes encoding GCC or uroguanylin included a larger number of cells undergoing apoptosis compared to levels observed in tissue from irradiated wild type mice. Experiments also showed cGMP supplementation ameliorated the level of apoptosis in irradiated intestinal tissue of mice lacking genes encoding GCC or uroguanylin but not in wild type mice.

Hendry et al. Radiation Research 1997148(3):254-9 report that radiation induced apoptosis of intestinal cells does not correlate with the survival rate of clonogenic cells responsible for the recovery of epithelial cells of the intestine.

Komarova et al. Oncogene (2004) 23, 3265-3271 use p53 deficient mice to show that cell cycle arrest following irradiation prolongs survival by delaying crypt cells from entry into a mitotic catastrophe and fast death after being damaged by radiation. Arresting proliferation of crypt cells after irradiation enhances survival of epithelium of the small intestine. The cycle arrest is attributed to a protective role of p53 through its growth arrest rather than apoptotic function.

Kirsch et al, Science 2010 327:593-6 report that radiation induced gastrointestinal syndrome is apoptosis independent. Using genetically modified mice which have tissue specific suppression of apoptosis essential genes, the authors show that radiation induced gastrointestinal syndrome can proceed in the absence of a complete compliment of proteins required to undergo apoptosis, and therefore that radiation induced gastrointestinal syndrome is independent of the intrinsic apoptosis pathway. Deletion of p53 expression in epithelial cells sensitized irradiated mice to radiation induced gastrointestinal syndrome while overexpression of p53 was protective. The data show that p53 expression is linked to survival following high doses of ionizing radiation even in animals which lack other proteins essential to the intrinsic apoptosis pathway; radiation induced gastrointestinal syndrome is independent of apoptosis.

U.S. Ser. No. 14/114,272, which is incorporated in its entirety herein by reference, refers to compositions for and methods of protecting an individual from serious and possibly lethal effects associated with exposure to radiation and some toxic compounds, to compositions for and methods of protecting an individual from serious and possibly lethal side effects associated with cancer chemotherapy and radiation therapy and to compositions and methods that are particularly useful to protect the gastrointestinal (GI) tract from GI syndrome caused by radiation.

There remains a need for treatments which minimize the side effects chemotherapy and radiation therapy in order to increase patient comfort and to allow for an increase in dosage which would otherwise be prevented due to unacceptable levels of side effects. Potentiating the therapeutic efficacy for cancer treatment by prevention of the side effects of chemotherapy and radiation therapy and increasing susceptibility to cancer cells represents a major advance in the treatment of cancer. There remains a need to identify compositions and methods of preventing GI syndrome and reducing the severity of gastrointestinal side effects following exposure to toxic chemotherapy or radiation. There remains a need to protect gastrointestinal cells from damage by exposure to toxic chemotherapy or radiation leading to GI syndrome. There remains a need to reduce lethal effects of radiation and chemotherapy due to damage to gastrointestinal cells and increasing the tolerable levels of toxic chemotherapy and radiation in order to provide more effective therapy.

### SUMMARY OF THE INVENTION

Methods of treating individuals who have cancer identified as lacking functional guanylyl cyclase C are provided. The methods comprise administering to gastrointestinal cells in the individual who has been identified as having cancer which lacks functional guanylyl cyclase C, an amount of one or more guanylyl cyclase C agonist compounds sufficient to activate guanylyl cyclase C of the gastrointestinal cells and elevate intracellular cGMP in the gastrointestinal cells to a level that protects gastrointestinal cells from genotoxic damage. Activation of guanylyl cyclase C of the gastrointestinal cells results in elevation of intracellular cGMP in the gastrointestinal cells which causes arrest of cell proliferation of the gastrointestinal cells, and/or inhibition of DNA synthesis and prolongation of cell cycle of the gastrointestinal cells by imposing a G1-S delay and/or genomic integrity of the gastrointestinal cells to be maintained by enhanced DNA damage sensing and repair and thereby causes protection if the gastrointestinal cells from genotoxic damage caused by chemotherapy and/or radiation. Thus, reference to a level of intracellular cGMP in the gastrointestinal cells that protects gastrointestinal cells refers to that level which causes arrest of cell proliferation of the gastrointestinal cells, and/or inhibition of DNA synthesis and prolongation of cell cycle of the gastrointestinal cells by imposing a G1-S delay and/or genomic integrity of the gastrointestinal cells to be maintained by enhanced DNA damage sensing and repair, thereby rendering the gastrointestinal cells protected from genotoxic damage caused by chemotherapy and/or radiation. The method additionally provides the step of administering chemotherapy and/or radiation therapy to kill cancer cells that lack functional guanylyl cyclase C. The chemotherapy and/or radiation is administered when normal gastrointestinal cells have been rendered to protected from genotoxic damage cell by the effects of elevated intracellular cGMP in the gastrointestinal cells.

Methods of treating individuals who have primary colorectal cancer which lacks functional p53 in an individual are provided. Methods may include the step of identifying such individual. The methods comprise the step of administering to gastrointestinal cells in the individual who has been identified as having primary colorectal cancer which lacks functional p53, an amount of one or more guanylyl cyclase C agonist compounds sufficient to activate guanylyl cyclase C of the gastrointestinal cells and elevate intracellular cGMP in the gastrointestinal cells to a level that protects gastrointestinal cells from genotoxic damage. The method further provides administering chemotherapy and/or radiation therapy to kill primary colorectal cancer cells that lack functional p53. The chemotherapy and/or radiation is administered when normal gastrointestinal cells have been rendered protected from genotoxic damage cell by the effects of elevated intracellular cGMP in the gastrointestinal cells.

Methods of treating individuals who have cancer are provided. The methods comprise administering to intestinal stem cells in the individual an amount of one or more guanylyl cyclase C agonist compounds sufficient to activate guanylyl cyclase C of the intestinal stem cells and elevate intracellular cGMP in the intestinal stem cells to a level that that causes an increase in intestinal stem cell number and a shift of relative balance of intestinal stem cells to increase intestinal stem cells with a Lgr5+ active phenotype and to decrease intestinal stem cells with a Bmi1+ reserve phenotype. The methods also provide administering chemotherapy and/or radiation therapy to kill cancer cells when intestinal stem cell number is increased and relative balance of intestinal stem cells is shifted to increase intestinal stem cells with a Lgr5+ active phenotype and to decrease intestinal stem cells with a Bmi1+ reserve phenotype. Fewer and less severe gastrointestinal side effects occur when the chemotherapy and/or radiation is administered when intestinal stem cell number is increased and relative balance of intestinal stem cells is shifted to increase intestinal stem cells with a Lgr5+ active phenotype and to decrease intestinal stem cells with a Bmi1+ reserve phenotype.

Methods of treating individuals who have been identified as having cancer which lacks functional p53 are provided. In the methods the individual as having cancer which lacks functional p53 are identified. One or more compounds selected from the group consisting of: Guanylyl cyclase A (GCA) agonists (ANP, BNP), Guanylyl cyclase B (GCB) agonists (CNP), Soluble guanylyl cyclase activators (nitric oxide, nitro-vasodilators, protoprophyrin IX, and direct activators), PDE Inhibitors, MRP inhibitors, cyclic GMP and cGMP analogues are administered to gastrointestinal cells in the individual in an amount sufficient to elevate intracellular cGMP in normal cells and protect the normal cells from genotoxic effects of chemotherapy and/or radiation. Chemotherapy and/or radiation therapy are administered to kill cancer cells. The chemotherapy and/or radiation is administered when the normal cells are protected from genotoxic effects of chemotherapy and/or radiation.

Compositions comprising a guanylyl cyclase C agonist in an amount effective to protect intestinal tissue against radiation or chemotherapy are disclosed as are methods of preventing GI syndrome or RIGS and of for reducing side effects in cancer patient undergoing radiation or chemotherapy.

Some embodiments of the invention relate to methods of reducing gastrointestinal side effects in individuals undergoing chemotherapy or radiation therapy to treat cancer. The individuals may have cancer that is guanylyl cyclase C deficient, p53 deficient or both. Methods of treating primary colorectal cancer that is p53 deficient are provided. The methods comprise the steps of, prior to administration of chemotherapy or radiation to the individual, administering to the individual an amount of one or more compounds that elevates intracellular cGMP levels in gastrointestinal cells sufficient to arrest cell proliferation of gastrointestinal cells and/or maintain genomic integrity by enhanced DNA damage sensing and repair for a period sufficient to increase survival of gastrointestinal cells and reduce severity of chemotherapy or radiation therapy side effects. In some embodiments, reduction of side effects occurs by activation of guanylyl cyclase C in intestinal stem cells.

### BRIEF DESCRIPTION OF THE DRAWINGS

FIGS. 1A, 1B, 1C, 1D, 1E, 1F, 1G, 1H and 1I disclose data from experiments showing GUCY2C silencing amplifies RIGS. (1A) Gucy2c−/− mice are more susceptible to death, compared to Gucy2c−/− mice, induced by high dose (15 Gy) whole body irradiation (TBI, Kaplan-Meier analysis, ***p<0.001; n=34 Gucy2c+/+ mice, n=39 Gucy2c−/− mice). (1B) Mortality from low dose (8 Gy) TBI reflected hematopoictic toxicity, which was abrogated by bone marrow transplantation (BMT). In contrast, mortality from high dose TBI (15 Gy) reflected both hematopoietic and GI toxicity which could not be rescued by BMT [Kaplan-Meier analysis, ***p<0.001 between Gucy2c+/+ mice (n=11) and Gucy2+/+ mice with BMT (n=5) following low dose (8 Gy) TBI; p<0.05 (not significant) between Gucyc2c+/+ mice (n=21) and Gucy2c+/+ mice with BMT (n=15) following high dose TBI], Following 18 Gy STBI, Gucy2c−/− mice were more susceptible to diarrhea (1C, Chi-square test, two sided, *p<0.05), mortality (1D, Kaplan-Meier analysis, **p<0.01), weight loss (1E, Frailty model analysis, *p<0.05), intestinal bleeding (1F, fecal occult blood; FOB; Cochran-Mantel-Haenszel test, *p<0.05), debilitation (1G, untidy fur; Cochran-Mantel-Haenszel test, *p<0.05), and stool water accumulation [1H, Loess smoothing curves with 95% confidence bands and comparison of area under curve (AUC), *p<0.05; dashed line indicates stool water content before irradiation]. (I-L) Gucy2c−/− mice were more susceptible to radiation-induced GI injury in both small (I-J) and large (1K-1L) intestines quantified by crypt enumeration post irradiation (15 Gy TBI), compared to Gucy2c+/+ mice (ANOVA, *p<0.05, **p<0.01, n>3 Gucy2c+/+ and Gucy2c−/− mice, respectively, at each time point). Bars in low power images represent 500 μm, and in high power images 50 μm, in 1I and 1K. n=34 Gucy2c+/+ mice, n=35 Gucy2c−/− mice in 1C-1E and 1G; n=13 Gucy2c+/+ mice, n=13 Gucy2c−/− mice in 1F; n=26 Gucy2c+/+ mice and n=28 Gucy2c−/− mice in 1H.

FIGS. 2A, 2B, 2C, 2D, 2E and 2F disclose data from experiments that show GUCY2C hormone axis is preserved in RIGS. (2A-2F) TBI (15 Gy) did not significantly alter the relative expression of (2A) GUCY2C, (2B) guanylin (GUCA2A), or (2C) uroguanylin (GUCA2B) mRNA or (2D) GUCY2C, (2E) GUCA2A, or (2F) GUCA2B protein in jejunum over time [n=4-8 per time point; p>0.05 (not significant) for mRNA or protein by ANOVA]. (2G-2I) Representative immunofluorescence images for the expression of GUCY2C, GUCA2A, and GUCA2B before and 48 h after 15 Gy TBI [green, GUCY2C; red, hormone (GUA2A, GUCA2B); blue, DAPI]. Bar represents 50 μm.

FIGS. 3A, 3B, 3C, 3D, 3E, 3F, 3G, 3H, 3I, 3J and 3K disclose data from experiments showing Oral ST selectively opposes RIGS. (3A-3F) Gucy2c+/+ mice preconditioned with oral ST for 14 d prior to and following 18 Gy STBI (on d 0), exhibited a lower incidence of diarrhea (3A) induced by STBI, compared to mice treated with control peptide (CP) (Fisher's exact test, two sided, *p<0.05). Similarly, following 18 Gy STBI, ST improved (3B) diarrhea-free survival (Kaplan-Meier survival analysis, **p<0.01); (3C) weight loss and weight recovery (Frailty model analysis, *p<0.05); (3D) FOB and (3E) untidy fur (Cochran-Mantel-Haenszel test, *p<0.05); and (3F) stool water content [Loess smoothing curves with 95% confidence bands and comparison of area under curve (AUC), ***p<0.001; dashed line, stool water content before irradiation]. (3G-3H) ST reduced radiation-induced intestinal damage 15 d following STBI, reflected by (3G) gross morphology, with lower hyperemia, edema, and unformed stool, and (3H) histology, without disruption of normal crypt-villus architecture quantified by crypt depth, stromal hypertrophy reflected by intestinal transmural thickness and lymphocytic infiltration (Fisher's exact test, two sided, *p<0.05 in 3G; t-test, two sided, ***p<0.001 in 3H; n=4 CP-treated mice, n=5 ST-treated mice). Bar in 3H represents 100 μm. (3I) Oral ST did not alter radiation responses by subcutaneous thymoma or melanoma (t test, two-sided, p>0.05; dashed line, original tumor size). (3J-3K) Chronic (>2 weeks) oral ST did not produce diarrhea (3J) or growth retardation (3K) (p>0.05, ANOVA). In 3A-3F and 3I-3K, n=9 CP-treated mice, n=9 ST-treated mice.

FIGS. 4A, 4B, 4C, 4D, 4E, 4F, 4G and 4H disclose data from experiments showing GUCY2C signaling amplifies p53 response to RIGS by disrupting p53-Mdm2 interaction. (4A) Silencing GUCY2C did not affect apoptosis induced by 15 Gy TBI in small intestine and colon (t-test, two sided, *p<0.05; Gucy2c−/− mice, n>3 and Gucy2c+/+ mice, n≥3 at each time point). Oral ST improved (4B) diarrhea-free survival [Kaplan-Meier analysis, **p<0.01 between p53int+/+ mice treated with CP (n=17) or ST (n=16); p>0.05 between p53int−/− mice treated with CP (n=11) or ST (n=11)]; (4C) weight [Frailty model analysis, *p<0.05 between p53int+/+ mice treated with CP (n=17) or ST (n=16); p>0.05 between p53int−/− mice treated with CP (n=11) or ST (n=11)]; and (4D) FOB and (4E) untidy fur [Cochran-Mantel-Haenszel test, *p<0.05 between p53int+/+ mice treated with CP (n=17) or ST (n=16); p>0.05 between p53int−/− mice treated with CP (n=11) or ST (n=11)] following 18 Gy STBI in p53int+/+, but not p53int−/−, mice. (4F) Oral ST promoted p53 phosphorylation in small intestine 7 d after 18 Gy STBI (t-test, two sided, *p<0.05, n=6 in CP and n=6 in ST treated mice). Bar represents 50 μm. (4G) 8-Br-cGMP increased total and phosphorylated p53 in response to 5 Gy radiation in HCT116 human colon carcinoma cells (n≥3 in each treatment group; *p<0.05, **p<0.01, ANOVA). (4H) 8-Br-cGMP increased p53 activation in HCT116 cells in response to 5 Gy radiation by disrupting p53-Mdm2 interaction, quantified by immunoprecipitation (IP) and Western Blot (WB) with antibodies to p53 or Mdm2 (n≥3 in each group; *p<0.05, **p<0.01, ANOVA). Mouse and rabbit IgG were used as isotype controls in (4H).

FIGS. 5A, 5B, 5C, SD, SE, 5F, 5G and 5H disclose data from experiments showing GUCY2C signaling requires p53 to oppose mitotic catastrophe. (5A) Oral ST reduced DNA double strand breaks (γ-H2AX; t-tests, two sided, ***p<0.001, n=4 mice treated with CP treated, n=5 mice treated with ST; >200 crypts were examined in each mouse) and (5B) abnormal mitotic Orientation [(%) of metaphase plates oriented non-orthogonally to the crypt surface axis)] (t-tests, two sided, *p<0.05; n=4 mice treated with CP treated, n=5 mice treated with ST; >50 mitotic figures were evaluated in each mouse intestine) following 18 Gy STBI (red, β-catenin; green, γ-H2AX; blue, DAPI). Bars represent 50 μm. (5C) Radiation (5 Gy)-induced anaphase bridging, a marker of abnormal mitosis quantified by the anaphase bridge index (ABI), in wild type (parental) and p53-null (p53−/−) HCT116 human colon carcinoma cells, was reduced by pretreatment with a cell-permeant analogue of cGMP in a p53-dependent fashion. Representative images of ABI: i, normal mitosis without anaphase bridge, ii-iii, abnormal mitoses with anaphase bridges (Chi-square tests, two sided, *p<0.05; >100 cells in anaphase were examined in each group). (5D) Radiation (5 Gy)-induced aneuploidy, quantified by centrosome enumeration, in parental and p53-null HCT116 cells, was reduced in a p53-dependent fashion by pretreatment with 8-Br-cGM P. Representative images of ploidy: i, normal diploidy, ii, abnormal diploidy, iii, triploidy, iv, tetraploidy. (red, α/β-tubulin; green, γ-tubulin; purple, DAPI) (Chi-square tests, two sided, ***p<0.001, >200 mitotic cells were examined in each group). (5E) Cytogenetic toxicity induced by increasing doses of radiation, quantified by colony formation, in parental and p53-null HCT116 cells was reduced in a p53-dependent fashion by pretreatment with 8-Br-cGMP [Pairwise comparison of isotherm slopes, *p<0.05: HCT116 cells treated with cGMP compared to the three other groups including HCT116 cells treated with PBS, HCT116 p53-null cells treated with PBS or cGMP; p>0.05 (not significant) between any two of these three latter groups].

FIG. 6 panels A-L show data from Example 2. Gucy2c maintains the balance of Lgr5⁺ and Bmi1⁺ cells in crypts. (A-B) Enumeration of CBC ISCs in small intestinal sections using transmission electron microscopy (n=3 mice, ≥30 crypts/mousc). (C) Ex vivo enteroid forming capacity of crypts from Gucy2c^{-/-} mice relative to Gucy2c^{+/+} mice. (D) Quantification of Lgr5⁺ (GFP^{High}) cells by flow cytometry in crypts from Lgr5-EGFP-Cre-Gucy2c^{+/+} and Gucy2c^{-/-} mice. (E-F) Enumeration of Lgr5⁺GFP⁺ cells in intestinal crypts by EGFP IF (≥4 sections/mouse). (G-H) Crypt Lgr5⁺ cell lineage tracing events expressed as a percent of total crypts per section (≥4 sections/mouse). (I-J) Bmi1⁺ cells per intestinal section (≥4 sections/mouse). (K-L) Quantification of Bmi1 expressed in isolated crypt lysates, relative to β-actin (n=5 Gucy2c^{+/+}, 4 Gucy2c^{-/-}). *, p<0.05; ***, p<0.001. Bars in E and G represent 50 μm; bar in I represents 20 μm.

FIG. 7 panels A-G. Functional GUCY2C is expressed in Lgr5⁺ cells. (A) Flow sorting of GFP⁺ and GFP⁻ cells from crypts of Lgr5-EGFP-Cre-Gucy2c^{+/+} mice produced populations of active stem (Lgr5^{High}/SI^{Low}) and differentiated (Lgr5^{Low}/SI^{High}) cells (n=3). (B) GUCY2C mRNA expression, quantified by RT-PCR, was compared in Lgr5^{High}/SI^{Low} and Lgr5^{Low}/SI^{High} cells. (C) GUCY2C (green), immunofluorescence in GFP⁺ (red) cells. β-catenin (cyan) highlights individual cells and DAPI (blue) highlights nuclei. (D) ST activates GUCY2C and downstream VASP serine 239 phosphorylation (P-VASP-239) (white) in GFP⁺ (green) cells in Gucy2c^{+/+}, but not Gucy2c^{-/-}, mice. β-catenin (red) highlights individual cells and DAPI(blue) highlights nuclei. (E-F) 8Br-cGMP reconstitutes levels of (E) Lgr5⁺GFP⁺ and (F) Bmi1⁺ cells in crypts of Gucy2c^{-/-} mice that are comparable to those in Gucy2c^{+/+} mice. (G) Linaclotide enhances the enteroid-forming capacity of crypts in Gucy2c^{-/-} mice relative to Gucy2c^{+/+} mice. *, p<0.05; ns, not significant. Bar in C represents 50 μm; bar in D represents 20 μm.

FIG. 8 panels A-F. GUCY2C opposes ER stress balancing active and reserve ISCs. (A, B) Quantification of crypt ER stress marker expression, relative to tubulin, in Gucy2c^{+/+} and Gucy2c^{-/-} mice (n=3). (C, D) Grp78 (BiP) expression in crypts (*) of Gucy2c^{+/+} mice, and Gucy2c^{-/-} mice before and after treatment with TUDCA or 8Br-cGMP. (E, F) Quantification of crypt Lgr5⁺GFP⁺ and Bmi1⁺ cells in Gucy2c^{+/+} and Gucy2c^{+/+} mice following oral TUDCA for 3 d. *, p<0.05; ***, p<0.001; ns, not significant. Bar in C represents 20 μm.

FIG. 9 panels A-D. Maintenance of ISCs by GUCY2C contributes to regenerative responses following radiation-induced intestinal injury. Lgr5-EGFP-Cre-Gucy2c^{+/+} and −Gucy2c^{-/-} mice received 10 Gy of irradiation and the dynamics of (A-B) viable crypts, (C) GFP⁺Lgr5⁺ active stem cells, and (D) Bmi1⁺ reserve stem cells were quantified over the subsequent 3 days. Bar in B represents 100 μm.

FIG. 10 Enumeration of intestinal crypts containing Lgr5⁺GFP⁺ cells (≥4 sections/mouse) in Lgr5-EGFP-Cre-Gucy2c^{+/+} and Lgr5-EGFP-Cre-Gucy2c^{-/-} mice.

FIG. 11 Enumeration of intestinal crypts containing Bmi1⁺ cells (≥4 sections/mouse) in Gucy2c^{+/+} and Gucy2c^{-/-} mice.

### DESCRIPTION OF PREFERRED EMBODIMENTS

The cell signaling molecule cyclic GMP can prevent genotoxic damage to cells through a p53-dependent mechanism. Compounds that promote or otherwise result in accumulation of cGMP can therefore be administered to protect the cells of an individual from genotoxic damage caused by chemotherapy or radiation. In cases in which the individual is being treated for cancer with chemotherapy and/or radiation, compounds that promote or otherwise result in accumulation of cGMP are particularly useful if the cancer cells lack functional p53. In such cases, administration of such compounds protects the cells of an individual from genotoxic damage caused by chemotherapy or radiation while not protecting the cancer cells from genotoxic damage. In such methods, the individual is identified as having a tumor that lacks functional p53 and is then administered the compounds to protect normal cells.

GCC agonists are well known. When a GCC agonists interacts with cells that have the cellular receptor GCC (also referred to as GUCY2C), activation of GCC leads to accumulation of cGMP in the cell. Thus, GCC agonists can be administered to protect the GCC expressing cells of an individual from genotoxic damage caused by chemotherapy or radiation. In cases in which the individual is being treated for cancer with chemotherapy and/or radiation, GCC agonists are particularly useful to treat cancer cells that lack GCC. In such cases, administration of GCC agonists protects the cells of an individual from genotoxic damage caused by chemotherapy or radiation while not protecting the GCC deficient cancer cells from genotoxic damage. In such methods, the individual is identified as having a tumor that lacks functional GCC and is then administered the GCC agonist compounds to protect normal cells.

In the case of GCC deficient tumors and GCC agonists, this method is particularly useful. GCC is primarily expressed in normal intestinal cells. In such intestinal cells, the GCC extracellular portion of the protein is present on the side of the cells that make up the inside of the intestine. Oral administration of GCC agonist delivers the GCC agonist to the GCC of the intestinal cells and the intestinal cells accumulate cGMP. The intestinal cells are thereby protected from radiation and chemotherapy. This method is particularly useful in the treatment of non-GCC expressing cancers.

Most colorectal cancers express GCC as do some cancers of other alimentary canal organs and tissues such as stomach, esophageal and pancreatic cancers for example. While most colorectal cancer cells express GCC, some colorectal cancer cells lack GCC. Such GCC deficient phenotype may be correlated to particularly aggressive and difficult to treat colorectal cancers. In the case of cancer of organs or tissues that have cancer cells that are known to typically or sometimes express GCC, such as colorectal cancers express GCC, cancers of other alimentary canal organs and tissues such as stomach, esophageal and pancreatic cancers, methods may include a step of testing the tumor for GCC expression to identify the cancer as being GCC deficient and then administering GCC agonist to normal GCC expressing intestinal cells in order to activate GCC in such normal intestinal cells to bring about accumulation cGMP. Following such treatment with GCC agonist, the individual may undergo radiation and/or chemotherapy to treat the GCC deficient cancer while protecting the normal intestinal cells from damage.

In cases in which normal intestinal tissue is protected using GCC agonist, the normal intestine is protected from chemotherapy that operates by a genotoxic mechanism as well as radiation therapy. Patients undergoing abdominopelvic radiation are particularly prone to genotoxic damage to there intestines by such radiation therapy and protection of normal intestinal tissue using GCC agonist is particularly useful in the treatment of such patients.

Protection of normal cells allows for higher doses of radiation to be used and/or minimizes unpleasant and possible lethal side effects of radiation therapy due to damage to normal intestinal tissue exposed to such radiation during a radiation treatment directed to the abdominopelvic region.

In some embodiments, in addition to protection using GCC agonists, the patient may additionally be treated with other compounds that promote GCC accumulation provided that if such compounds are delivered systemically, the cancer is p53 defective.

Of the known GCC agonists, the heat stable enterotoxin ST, and the US FDA approved drugs linaclotide (SEQ ID NO:59) and plecanatide (SEQ ID NO:60) are particularly useful to protect normal intestinal epithelium in patients undergoing cancer therapy that employs genotoxic agents (e.g., radiation, chemotherapy), particularly when the cancer is GCC deficient, that is cancers that do not express GCC, such as most colorectal cancers, and some cancers of other alimentary canal organs and tissues such as stomach, esophageal and pancreatic cancers. Methods of detecting GCC expression in a tumor sample are well known and prior to treating a patient with a GCC agonist administered orally or by other means directly to the intestine in order to protect the intestine, the patient may be first identified as having a GCC deficient (lacking GCC function) cancer by analyzing a tumor sample to confirm the absence of GCC expression. If the tumor is also identified as being p53 deficient (lacking p53 function), other compounds may be used to induce accumulation of GCC in normal tissues alone or in combination with GCC agonists to protect normal intestine.

To protect normal intestine using GCC agonists, it is preferred that the normal intestine be exposed to the GCC agonist for a period of time sufficient to allow for cGMP accumulation to protective levels. In some embodiments, such accumulation may take 1-14 days, 3-10 days, 4, 5, 6, 7 8 or 9 days. GCC agonists, such as for example the heat stable enterotoxin ST, guanylin, uroguanylin and the US FDA approved drugs linaclotide (SEQ ID NO:59) and plecanatide (SEQ ID NO:60) may not be effective to induce accumulation ofcGMP sufficient to protect normal intestinal cells. When treating patients, the effectiveness of GCC agonist may be assessed by monitoring changes in bowel activity in patients being administered GCC agonist. Patients who experience changes in bowel activity after initiation of GCC agonist administration are likely going to be protected. Those that do not experience changes in bowel movement are likely to be non-responders and will not be protected. Some embodiments comprise the step of identifying the patient as being a patient with a GCC deficient cancer such as non-alimentary cancers and some colorectal cancers, and some cancers of other alimentary canal organs and tissues such as stomach, esophageal and pancreatic cancers. Cancers whose treatment may involve abdominopelvic radiation include pancreas, liver, stomach, biliary system, peritoneum, bladder, kidney, ureter, prostate, ovaries, uterus and soft tissues of the abdomen and pelvis such as sarcomas. Protecting normal intestine from radiation using GCC agonist is particularly useful. Identifying the cancers as not expressing GCC may be useful if there is a chance the cancer will come into contact with the GCC agonist. The determination may be made by testing a sample of tumor to determine the presence of GCC or other evidence of GCC expression such as GCC mRNA. The method further comprising administering GCC agonist, such as the heat stable enterotoxin ST, or US FDA approved drug linaclotide (SEQ ID NO:59) or US FDA approved drug plecanatide (SEQ ID NO:60) in an amount effective to protect normal intestinal epithelium in patients undergoing cancer therapy that employs genotoxic agents (e.g., radiation, chemotherapy). The GCC agonist is preferably delivered orally. The GCC agonist delivery is continued provided bowel movement changes are observed to indicate that the patient is likely going to be protected. In some embodiments, the cancer is removed surgically prior to radiation. In such cases, the methods may include treating patients who have GCC+ cancers if the tumors are surgically removed before treating with abdominopelvic radiation.

In some embodiments, methods are provided for treating an individual who has been identified as having cancer which lacks functional guanylyl cyclase C. In some embodiments, the cancer that lacks functional guanylyl cyclase C is selected from the group consisting of: colorectal cancer which lacks functional guanylyl cyclase C, esophageal cancer which lacks functional guanylyl cyclase C, pancreatic cancer which lacks functional guanylyl cyclase C, liver cancer which lacks functional guanylyl cyclase C, stomach cancer which lacks functional guanylyl cyclase C, biliary system cancer which lacks functional guanylyl cyclase C, cancer of the peritoneum which lacks functional guanylyl cyclase C, bladder cancer which lacks functional guanylyl cyclase C, kidney cancer which lacks functional guanylyl cyclase C, cancer of the ureter which lacks functional guanylyl cyclase C, prostate cancer which lacks functional guanylyl cyclase C, ovarian cancer which lacks functional guanylyl cyclase C, uterus cancer which lacks functional guanylyl cyclase C and soft tissues of the abdomen and pelvis such as sarcomas which lack functional guanylyl cyclase C. In some embodiments, the methods provide the step of administering to gastrointestinal cells in the individual who has been identified as having cancer which lacks functional guanylyl cyclase C, an amount of one or more guanylyl cyclase C agonist compounds sufficient to activate guanylyl cyclase C of the gastrointestinal cells and elevate intracellular cGMP in the gastrointestinal cells to a level that protects gastrointestinal cells from genotoxic damage. Protection from genotoxic damage and gastrointestinal side effects caused by chemotherapy and radiation arises from the effect elevated intracellular cGMP in the gastrointestinal cells has on the cells. The elevated cGMP occurs because of activation of the guanylyl cyclase C. As a result, cell proliferation of the gastrointestinal cells is arrested, and/or DNA synthesis is inhibited and the cell cycle of the gastrointestinal cells is prolonged by imposing a G1-S delay, and/or genomic integrity of the gastrointestinal cells is maintained by enhanced DNA damage sensing and repair.

In some embodiments, the method comprises the step of identifying the individual as having cancer which lacks functional guanylyl cyclase C. In some embodiments, the lack of functional guanylyl cyclase C is determined by detecting the absence of guanylyl cyclase C or RNA that encodes guanylyl cyclase C in a sample of cancer cells from the individual. In some embodiments, the method comprises the step of identifying the individual as having cancer which lacks functional guanylyl cyclase C by detecting the absence of guanylyl cyclase C in a sample of cancer cells from the individual by contacting the sample of cancer cells with a reagent that binds to guanylyl cyclase C and detecting the absence of binding of the reagent to the sample cancer cells. In some embodiments, the method comprises the step of identifying the individual as having cancer which lacks functional guanylyl cyclase C by detecting the absence of guanylyl cyclase C in a sample of cancer cells from the individual by contacting the sample of cancer cells with a reagent that binds to guanylyl cyclase C and detecting the absence of binding of the reagent to the sample cancer cells, wherein the reagent is an anti-guanylyl cyclase C or a guanylyl cyclase C ligand. In some embodiments, the method comprises the step of identifying the individual as having cancer which lacks functional guanylyl cyclase C by detecting the absence of RNA that encodes guanylyl cyclase C in a sample of cancer cells from the individual by performing PCR on mRNA from the sample of cancer cells using PCR primers that amplify RNA that encodes guanylyl cyclase C and detecting the absence of amplified RNA in the sample cancer cells or by contacting an oligonucleotide with mRNA from the sample of cancer cells wherein the oligonucleotide has a sequence that hybridizes to RNA that encodes guanylyl cyclase C and detecting the absence of oligonucleotide hybridized to mRNA from the sample of cancer cells.

In some embodiments, the methods further comprising identifying the cancer which lacks guanylyl cyclase C as also lacking functional p53. In such embodiments, one or more active agents selected from the group consisting of: Guanylyl cyclase A (GCA) agonists (ANP, BNP), Guanylyl cyclase B (GCB) agonists (CNP), Soluble guanylyl cyclase activators (nitric oxide, nitro-vasodilators, protoprophyrin IX, and direct activators), PDE Inhibitors, MRP inhibitors, cyclic GMP and cGMP analogues may be administered to the individual to protect normal cells by increasing intracellular cGMP. In some such embodiments, the cancer is identified as lacking functional p53 by detecting the absence of p53 or RNA that encodes p53 in a sample of cancer cells from the individual.

In some embodiments, methods are provided for treating an individual who has primary colorectal cancer which lacks functional p53. The methods may comprise administering to gastrointestinal cells in the individual who has been identified as having primary colorectal cancer which lacks functional p53, an amount of one or more guanylyl cyclase C agonist compounds sufficient to activate guanylyl cyclase C of the gastrointestinal cells and elevate intracellular cGMP in the gastrointestinal cells to a level that protects gastrointestinal cells from genotoxic damage. The methods further provide administering chemotherapy and/or radiation therapy to kill primary colorectal cancer cells that lack functional p53. The chemotherapy and/or radiation administration is performed when normal gastrointestinal cells are protected from genotoxic damage cell by the effects of elevated intracellular cGMP in the gastrointestinal cells. Some embodiments provide the step of identifying the individual as having primary colorectal cancer which lacks functional p53.

Some methods are provided for treating an individual who has cancer by administering one or more guanylyl cyclase C agonist compounds to intestinal stem cells in the individual. The guanylyl cyclase C agonist compounds are administered in an amount of sufficient to activate guanylyl cyclase C of the intestinal stem cells and elevate intracellular cGMP in the intestinal stem cells to a level that that causes an increase in intestinal stem cell number and a shift of relative balance of intestinal stem cells to increase intestinal stem cells with a Lgr5+ active phenotype and to decrease intestinal stem cells with a Bmi1+ reserve phenotype. Chemotherapy and/or radiation therapy is administered to kill cancer cells. By increasing in intestinal stem cell numbers and shift from active to reserve phenotype and then treating with chemotherapy or radiation when the stems cells are as such, the gastrointestinal track regenerates and heals more effectively.

Some methods provide administering chemotherapy. Some methods provide administering radiation. Some methods provide administering administered abdominopelvic radiation.

In some embodiments, the one or more GCC agonist compounds is a GCC agonist peptide. In some embodiments the one or more GCC agonist compounds is selected from the group consisting of SEQ ID NOs:2, 3 and 5-60. In some embodiments the one or more GCC agonist compounds is selected from guanylin, uroguanylin, SEQ ID NO:59, SEQ ID NO:60 and combinations thereof. In some embodiments, the GCC agonist compound is administered to gastrointestinal cells or intestinal stem cells by oral administration of the one or more GCC agonist compounds to the individual. In some embodiments, the GCC agonist compound is administered by oral administration in a controlled release composition.

In some embodiments, the GCC agonist compound is administered to an individual 24 hours to 48 hours to 72 hours to 96 hours prior to administering to said individual chemotherapy or radiation an amount sufficient to treat cancer. In some embodiments, the GCC agonist compound is administered to the individual daily for 2, 3, 4, 5, 6, 7, 8, 9, 10, 11, 12, 13 or 14 days. In some embodiments, the GCC agonist compound is administered in multiple doses.

In some embodiments, tumor is surgically removed from the individual prior to administration of the guanylyl cyclase C agonist.

Since not everyone will respond to the GCC agonist compound by creating conditions in which the gastrointestinal cells are protected, individuals may be identified as responding to protective action of guanylyl cyclase C agonist compound by detecting changes in bowel movements of the individual following administration of the guanylyl cyclase C agonist. If the individual being administered guanylyl cyclase C agonist experiences changes in bowel movements in response to guanylyl cyclase C agonist, the methods can continue as described. Failure to respond suggests the individual is less likely to benefit and the methods may be discontinued.

Some methods are provided for treating individual who have been identified as having cancer which lacks functional p53. Such methods may comprise the step of identifying the individual as having cancer which lacks functional 53. Such methods may provide administering to gastrointestinal cells in the individual an amount of one or more compounds selected from the group consisting of: Guanylyl cyclase A (GCA) agonists (ANP, BNP), Guanylyl cyclase B (GCB) agonists (CNP), Soluble guanylyl cyclase activators (nitric oxide, nitro-vasodilators, protoprophyrin IX, and direct activators), PDE Inhibitors, MRP inhibitors, cyclic GMP and cGMP analogues in an amount sufficient to elevate intracellular cGMP in normal cells and protect the normal cells from genotoxic effects of chemotherapy and/or radiation. In such embodiments, chemotherapy and/or radiation therapy may be administered to kill cancer cells. In some embodiment, the method comprises the step of identifying the individual as having cancer which lacks functional p53 by detecting the absence of p53 or RNA that encodes p53 in a sample of cancer cells from the individual. In some embodiment, the method comprises administering one or more compounds selected from the group consisting of: Guanylyl cyclase A (GCA) agonists (ANP, BNP), Guanylyl cyclase B(GCB) agonists (CNP), Soluble guanylyl cyclase activators (nitric oxide, nitrovasodilators, protoprophyrin IX, and direct activators), PDE Inhibitors, MRP inhibitors, cyclic GMP and cGMP analogues is administered to said individual 24 hours prior to administering to said individual chemotherapy or radiation an amount sufficient to treat cancer; 48 hours prior to administering to said individual chemotherapy or radiation an amount sufficient to treat cancer; 72 hours prior to administering to said individual chemotherapy or radiation an amount sufficient to treat cancer; or 96 hours prior to administering to said individual chemotherapy or radiation an amount sufficient to treat cancer and/or Administering one or more compounds selected from the group consisting of: Guanylyl cyclase A (GCA) agonists (ANP, BNP), Guanylyl cyclase B (GCB) agonists (CNP), Soluble guanylyl cyclase activators (nitric oxide, nitrovasodilators, protoprophyrin IX, and direct activators), PDE Inhibitors, MRP inhibitors, cyclic GMP and cGMP analogues daily for 2, 3, 4, 5, 6, 7, 8, 9, 10, 11, 12, 13 or 14 days. In some embodiment, the one or more compounds selected from the group consisting of Guanylyl cyclase A (GCA) agonists (ANP, BNP), Guanylyl cyclase B (GCB) agonists (CNP), Soluble guanylyl cyclase activators (nitric oxide, nitrovasodilators, protoprophyrin IX, and direct activators), PDE Inhibitors, MRP inhibitors, cyclic GM P and cGMP analogues is administered in multiple doses. In some embodiments the tumor is surgically removed from the individual prior to administration of one or more compounds selected from the group consisting of: Guanylyl cyclase A (GCA) agonists (ANP, BNP), Guanylyl cyclase B (GCB) agonists (CNP), Soluble guanylyl cyclase activators (nitric oxide, nitrovasodilators, protoprophyrin IX, and direct activators), PDE Inhibitors, MRP inhibitors, cyclic GMP and cGMP analogues.

### Definitions

As used herein the terms “guanylyl cyclase A agonist” and “GCA agonists” are used interchangeably and refer to molecules which bind to guanylyl cyclase A on a cell surface and thereby induce its activity which results in cGMP accumulation within the cell.

As used herein the terms “guanylyl cyclase B agonist” and “GCB agonists” are used interchangeably and refer to molecules which bind to guanylyl cyclase B on a cell surface and thereby induce its activity which results in cGMP accumulation within the cell.

As used herein the terms “guanylyl cyclase C agonist” and “GCC agonists” are used interchangeably and refer to molecules which bind to guanylyl cyclase Con a cell surface and thereby induce its activity which results in cGMP accumulation within the cell.

As used herein the terms “soluble guanylyl cyclase activator” and “sGC activator” are used interchangeably and refer to molecules which bind to soluble guanylyl cyclase and thereby induce its activity which results in cGMP accumulation within the cell.

As used herein the terms “phosphodiesterase inhibitor” and “PDE inhibitors” are used interchangeably and refer to molecules which inhibit the activity of one or more forms or subtypes of the cGMP-hydrolyzing phosphodiesterase enzyme and thereby bringing about cGMP accumulation within the cell.

As used herein the terms “multidrug resistance-associated protein inhibitors” and “MRP inhibitors” are used interchangeably and refer to molecules which inhibit the activity of one or more forms or subtypes of the cGMP-transporting MRPs and thereby bringing about cGMP accumulation within the cell.

As used herein the term “effective amount” refers to the amount of compound(s) effective to result in the accumulation of intracellular cGMP levels to arrest cell proliferation of gastrointestinal cells and/or maintain genomic integrity by enhanced DNA damage sensing and repair for a period sufficient to reduce cell damage caused by chemotherapy or radiation sufficient to reduce the severity of side effects or prevent GUI syndrome and/or radiation sickness.

### Detecting GCC and Mutated Forms of p53

In situ imaging or in vitro screening and diagnostic compositions, methods and kits can be used determine if a tumor expresses guanylyl cyclase C (GCC). In vivo imaging is disclosed in U.S. Pat. No. 6,268,158, which is incorporated herein by reference in its entirety.

In vitro screening and diagnostic compositions, kits and methods for detecting GCC protein or RNA encoding GCC protein are disclosed in U.S. Pat. No. 6,060,037, which is incorporated herein by reference in its entirety.

In vitro screening and diagnostic compositions, kits and methods for detecting cells that contain mutated forms of p53 are disclosed in U.S. Pat. No. 5,552,283, which is incorporated herein by reference in its entirety.

cGMP

The intracellular accumulation of cGMP helps the cell maintain genomic integrity by enhanced DNA damage sensing and repair for a period sufficient to reduce cell damage caused by chemotherapy or radiation. The p53 protects irradiated cells from mitotic catastrophe by mediating arrest of cell proliferation to allow repair prior to cell division and thereby preventing cell death by mitotic catastrophe.

Side effects caused by radiation and chemotherapy including G syndrome can be reduced by p53 mediated cell arrest. Increasing intracellular cGMP levels results in enhanced p53 mediated cell arrest when such cells are exposed to lethal toxic chemotherapy or ionizing radiation insults. Increasing intracellular cGMP may be achieved by increasing its production and/or inhibiting its degradation or expulsion from cells. DNA damage repair may be promoted which in turn prevents the death of normal intestinal epithelial cells in response to chemotherapy and ionizing radiation insults.

Accordingly, in conjunction with administration of chemotherapy or radiation to individuals, individuals are administered an amount of one or more compounds that elevates intracellular cGMP levels in gastrointestinal cells sufficient to arrest cell proliferation of said gastrointestinal cells and/or maintain genomic integrity by enhanced DNA damage sensing and repair for a period sufficient to prevent GI syndrome. The one or more compounds that elevates intracellular cGMP levels may be administered prior to and/or simultaneous with and/or subsequent to administration of chemotherapy or radiation to the individual although typically, pretreatment one or more compounds that elevates intracellular cGMP levels is performed to ensure the p53 mediated cell protection is initiated before exposure to toxic chemicals or radiation.

While increases in cGMP levels protect intestinal cells following a toxic insult, cGMP may potentiate cell death in other cancer cells such as human breast, liver and prostate cancer. By inducing cGMP levels in intestinal epithelial cells to levels sufficient to maintain p53 mediated cell arrest prior to and in conjunction with administration of chemotherapy or radiation therapy, lethal side effects can be reduced, increased doses of chemotherapy or radiation therapy can be utilized and such therapy may be rendered more effective against cancer. When cGMP levels in intestinal epithelial cells are increased sufficient to result in a protection of such cells from toxins and radiation, chemotherapy and radiation therapy may proceed with reduced side effects and risks, even in some cases at higher doses which could not be tolerated absent the protection afforded by the elevated cGMP levels in the intestinal epithelial cells. Moreover, a simultaneous increase in cGMP in cancer cells in the patient may provide synergistic effects on chemotherapy and radiation therapy. The preconditioning of GI tract and targeted organs with treatments that result in intracellular accumulation of cGMP may dramatically increase the efficacy of chemotherapy or radiation therapy by broadening the therapeutic window and increasing the therapeutic index.

The intracellular increase of cGMP levels enhances p53 mediated cell survival in the intestine thereby limiting side effect of chemotherapy and radiation therapy in cancer patients. Thus, increasing intracellular cGMP levels in intestinal cells in particular can be affected prior to chemotherapy and radiation therapy at a time such that during the time when the patient is undergoing chemotherapy or and radiation therapy, the intestinal cells with are protected by p53 thus reducing typical side effects of chemotherapy and radiation therapy. To protect intestinal epithelial cells during chemotherapy and radiation therapy cGM P levels must be increased to an amount effective to enhance p53 mediated cell survival. Since radiation damage and the GI syndrome which results in severe and sometimes lethal side effects in patients receiving radiation is reduced by p53 and independent of apoptosis, the increased level cGMP levels must be sufficient to enhance p53 mediated cell survival.

On the other hand, an increase in intracellular cGMP may also potentiate cancer cell death in response to genetic insults by chemotherapy or ionizing radiation by promoting cell apoptosis in lung, prostate, breast, colorectal and liver cancer cells. Data suggest that cellular preconditioning with cGMP, or agents that result in increased levels of cGMP, in target organs and in the GI tract potentiate chemotherapy and radiation therapy (kill cancer cells) in the target organs while preventing GI tract (normal intestinal cell) damage.

The use of compounds which increase cGMP productions and/or compounds which inhibit cGMP degradation or export from the cell result in an increase in cGMP levels. When administered to the normal GI tract, the increase in cGMP levels serves to protect the cells from cell death which is associated with side effects associated with chemotherapy and radiation therapy, thereby increasing safety of these therapies. In addition, the reduction of side effects allows for toleration of increasing and more effective doses. When delivered to cancer cells such as lung, breast, prostate, colorectal, and liver cancers in order to increase cGMP levels, the cancer cells may become more susceptible to chemotherapy and radiation therapy thereby increasing the efficacy of the treatment.

Compounds which increase cGMP production include activators of guanylyl cyclases including three cellular receptor forms guanylyl cyclase A (GCA), guanylyl cyclase B (GCB) and guanylyl cyclase C (GCC) as well as soluble guanylyl cyclase (sGC).

Compounds which inhibit cGMP degradation and/or export from the include phosphodiesterase enzyme (PDE) inhibitors which inhibit PDE forms and subtypes involved in converting cGMP.

Compounds which inhibit cGMP export from the cell include multidrug resistance protein (MRP) inhibitors which inhibit MRP forms and subtypes involved in transport of cGMP.

These compounds can be used alone or in combinations of two or more to increase intracellular cGMP levels to protect cells of the intestines from cell death associated with chemotherapy and radiation therapy side effects and may render cancer cells more susceptible to cell death.

### GCC

GCC is the predominant guanylyl cyclase in the GI tract. Accordingly, the use of GCC activators or agonists is particularly effective to increase intracellular cGMP in the GI tract. The GCC activators include endogenous peptides guanylin and uroguanylin as well as heat stable enterotoxins produced by bacteria, such as E. coli STs. PDE inhibitors and MRP inhibitors are also known. In some embodiments, one or more GCC agonists is used. In some embodiments, one or more PDE inhibitors is used. In some embodiments, one or more MRP inhibitors is used. In some embodiments, a combination of one or more GCC agonists and/or one or more PDE inhibitors and/or one or more MRP inhibitors is used.

Activation of the cellular receptor guanylyl cyclase C (GCC), a protein expressed primarily in the GI tract, protects cells in the GI tract from dying in response to toxic chemotherapy or ionizing radiation insults. The activation of GCC leads to intracellular accumulation of cGMP which enhances p53 mediated cell survival. Many side effects caused by radiation and chemotherapy can be reduced by enhancing p53 mediated cell survival. By activating GCC, intracellular cGMP levels are increased resulting in enhanced p53 mediated cell survival when such cells are exposed to lethal toxic chemotherapy or ionizing radiation insults.

GCC is the intestinal epithelial cell receptor for the endogenous paracrine hormones guanylin and uroguanylin. Diarrheagenic bacterial heat-stable enterotoxins (STs) also target GCC. Hormone-receptor interaction between guanylin or uroguanylin and the extracellular domain of GCC or ST-receptor interaction between the peptide enterotoxin ST and the extracellular domain of GCC each activates the intracellular catalytic domain of GCC which converts GTP to cyclic GMP (cGMP). This cyclic nucleotide, as a second messenger, activates its downstream effectors mediating GCC's cellular effects. Increasing intracellular cGMP by activating guanylyl cyclase (including particulate and soluble forms) or by inhibiting cGMP degradation or expulsion by inhibitors of phosphodiesterases (PDEs) or multi-drug resistance associated proteins (MRPs), respectively, promotes DNA damage repair which in turn prevents the death of normal intestinal epithelial cells in response to chemotherapy and ionizing radiation insults.

Increases in cGMP levels such as those increases associated with GCC activation protect intestinal cells through p53 mediated cell survival following a toxic insult. Thus, activation of GCC can be affected prior to chemotherapy and radiation therapy at a time such that during the time when the patient is undergoing chemotherapy or and radiation therapy, the GCC activated intestinal cells are protected from typical side effect of chemotherapy and radiation therapy by p53 mediated cell survival. In addition to activation of GCC, protection of intestinal epithelial cells during chemotherapy and radiation therapy can be undertaken by increasing cGM P levels to an amount effective to enhance p53 mediated cell survival.

Since radiation damage and the GI syndrome which results in severe and sometimes lethal side effects in patients receiving radiation is independent of apoptosis and can be mitigated by p53, the level of GCC activation or other increase in cGMP levels must be sufficient to enhance p53 mediated cell survival.

Administration of a GCC agonist refers to administration of one or more compounds that bind to and activate GCC.

Guanylyl cyclase C (GCC) is a cellular receptor expressed by cells lining the large and small intestines. The binding of GCC agonists to GCC in the gastrointestinal track is known to activate GCC, leading to an increase in intracellular cGMP, which results in activation of downstream signaling events.

### GCC Agonists

GCC agonists are known. Two native GCC agonists, guanylin and uroguanylin, have been identified (see U.S. Pat. Nos. 5,969,097 and 5,489,670, which are each incorporated herein by reference. In addition, several small peptides, which are produced by enteric pathogens, are toxigenic agents which cause diarrhea (see U.S. Pat. No. 5,518,888, which is incorporated herein by reference). The most common pathogen derived GCC agonist is the heat stable enterotoxin produced by strains of pathogenic E. coli. Native heat stable enterotoxin produced by pathogenic E. coli is also referred to as ST. A variety of other pathogenic organisms including Yersinia and Enterobacter, also make enterotoxins which can bind to guanylyl cyclase C in an agonistic manner. In nature, the toxins are generally encoded on a plasmid which can “jump” between different species. Several different toxins have been reported to occur in different species. These toxins all possess significant sequence homology, they all bind to ST receptors and they all activate guanylate cyclase, producing diarrhea.

ST has been both cloned and synthesized by chemical techniques. The cloned or synthetic molecules exhibit binding characteristics which are similar to native ST. Native ST isolated from E. coli is 18 or 19 amino acids in length. The smallest “fragment” of ST which retains activity is the 13 amino acid core peptide extending toward the carboxy terminal from cysteine 6 to cysteine 18 (of the 19 amino acid form). Analogues of ST have been generated by cloning and by chemical techniques. Small peptide fragments of the native ST structure which include the structural determinant that confers binding activity may be constructed. Once a structure is identified which binds to ST receptors, non-peptide analogues mimicking that structure in space are designed.

U.S. Pat. Nos. 5,140,102 and 7,041,786, and U.S. Published Applications US 2004/0258687 A1 and US 2005/0287067 A1 also refer to compounds which may bind to and activate guanylyl cyclase C.

SEQ ID NO:1 discloses a nucleotide sequence which encodes 19 amino acid ST, designated ST Ia, reported by So and McCarthy (1980) Proc. Natl. Acad. Sci. USA 77:4011, which is incorporated herein by reference.

The amino acid sequence of ST Ia is disclosed in SEQ ID NO:2.

SEQ ID NO:3 discloses the amino acid sequence of an 18 amino acid peptide which exhibits ST activity, designated ST I*, reported by Chan and Giannella (1981) J. Biol. Chem. 256:7744, which is incorporated herein by reference.

SEQ ID NO:4 discloses a nucleotide sequence which encodes 19 amino acid ST, designated ST Ib, reported by Mosely et al. (1983) Infect. Immun. 39:1167, which is incorporated herein by reference.

The amino acid sequence of ST Ib is disclosed in SEQ ID NO:5.

A 15 amino acid peptide called guanylin which has about 50% sequence homology to ST has been identified in mammalian intestine (Currie, M. G. et al. (1992) Proc. Natl. Acad Sci. USA 89:947-951, which is incorporated herein by reference). Guanylin binds to ST receptors and activates guanylate cyclase at a level of about 10- to 100-fold less than native ST. Guanylin may not exist as a 15 amino acid peptide in the intestine but rather as part of a larger protein in that organ. The amino acid sequence of guanylin from rodent is disclosed as SEQ ID NO:6.

SEQ ID NO:7 is an 18 amino acid fragment of SEQ ID NO:2. SEQ ID NO:8 is a 17 amino acid fragment of SEQ ID NO:2. SEQ ID NO:9 is a 16 amino acid fragment of SEQ ID NO:2. SEQ ID NO:10 is a 15 amino acid fragment of SEQ ID NO:2. SEQ ID NO:11 is a 14 amino acid fragment of SEQ ID NO:2. SEQ ID NO:12 is a 13 amino acid fragment of SEQ ID NO:2. SEQ ID NO:13 is an 18 amino acid fragment of SEQ ID NO:2. SEQ ID NO:14 is a 17 amino acid fragment of SEQ ID NO:2. SEQ ID NO:15 is a 16 amino acid fragment of SEQ ID NO:2. SEQ ID NO:16 is a 15 amino acid fragment of SEQ ID NO:2. SEQ ID NO:17 is a 14 amino acid fragment of SEQ ID NO:2.

SEQ ID NO:18 is a 17 amino acid fragment of SEQ ID NO:3. SEQ ID NO:19 is a 16 amino acid fragment of SEQ ID NO:3. SEQ ID NO:20 is a 15 amino acid fragment of SEQ ID NO:3. SEQ ID NO:21 is a 14 amino acid fragment of SEQ ID NO:3. SEQ ID NO:22 is a 13 amino acid fragment of SEQ ID NO:3. SEQ ID NO:23 is a 17 amino acid fragment of SEQ ID NO:3. SEQ ID NO:24 is a 16 amino acid fragment of SEQ ID NO:3. SEQ ID NO:25 is a 15 amino acid fragment of SEQ ID NO:3. SEQ ID NO:26 is a 14 amino acid fragment of SEQ ID NO:3.

SEQ ID NO:27 is an 18 amino acid fragment of SEQ ID NO:5. SEQ ID NO:28 is a 17 amino acid fragment of SEQ ID NO:5. SEQ ID NO:29 is a 16 amino acid fragment of SEQ ID NO:5. SEQ ID NO:30 is a 15 amino acid fragment of SEQ ID NO:5. SEQ ID NO:31 is a 14 amino acid fragment of SEQ ID NO:5. SEQ ID NO:32 is a 13 amino acid fragment of SEQ ID NO:5. SEQ ID NO:33 is an 18 amino acid fragment of SEQ ID NO:5. SEQ ID NO:34 is a 17 amino acid fragment of SEQ ID NO:5. SEQ ID NO:35 is a 16 amino acid fragment of SEQ ID NO:5. SEQ ID NO:36 is a 15 amino acid fragment of SEQ ID NO:5. SEQ ID NO:37 is a 14 amino acid fragment of SEQ ID NO:5.

SEQ ID NO:27, SEQ ID NO:31, SEQ ID NO:36 AND SEQ ID NO:37 are disclosed in Yoshimura, S., et al. (1985) FEBS Lett. 181:138, which is incorporated herein by reference.

SEQ ID NO:38, SEQ ID NO:39 and SEQ ID NO:40, which are derivatives of SEQ ID NO:3, are disclosed in Waldman, S. A. and O'Hanley, P. (1989) Infect. Immun. 57:2420, which is incorporated herein by reference.

SEQ ID NO:41, SEQ ID NO:42, SEQ ID NO:43, SEQ ID NO:44 and SEQ ID NO:45, which are derivatives of SEQ ID NO:3, are disclosed in Yoshimura, S., et al. (1985) FEBS Lett. 181:138, which is incorporated herein by reference.

SEQ ID NO:46 is a 25 amino acid peptide derived from Y. enterocolitica which binds to the ST receptor.

SEQ ID NO:47 is a 16 amino acid peptide derived from V. cholerae which binds to the ST receptor. SEQ ID NO:47 is reported in Shimonishi, Y., et al. FEBS Lett. 215:165, which is incorporated herein by reference.

SEQ ID NO:48 is an 18 amino acid peptide derived from Y. enterocolitica which binds to the ST receptor. SEQ ID NO:48 is reported in Okamoto, K., et al. Infec. Immun. 55:2121, which is incorporated herein by reference.

SEQ ID NO:49, is a derivative of SEQ ID NO:5.

SEQ ID NO:50, SEQ ID NO:51, SEQ ID NO:52 and SEQ ID NO:53 are derivatives. SEQ ID NO:54 is the amino acid sequence of guanylin from human.

A 15 amino acid peptide called uroguanylin has been identified in mammalian intestine from opossum (Hamra, S. K. et al. (1993) Proc. Natl. Acad Sci. USA 90:10464-10468, which is incorporated herein by reference; see also Forte L. and M. Curry 1995 FASEB 9:643-650: which is incorporated herein by reference). SEQ ID NO:55 is the amino acid sequence of urognanylin from opossum.

A 16 amino acid peptide called uroguanylin has been identified in mammalian intestine from human (Kita, T. et al. (1994) Amer. J. Physiol. 266:F342-348, which is incorporated herein by reference; see also Forte L. and M. Curry 1995 FASEEB 9:643-650; which is incorporated herein by reference). SEQ ID NO:56 is the amino acid sequence of uroguanylin from human.

SEQ ID NO:57 is the amino acid sequence of proguanylin, a guanylin precursor which is processed into active guanylin.

SEQ ID NO:58 is the amino acid sequence of prouroguanylin, a uroguanylin precursor which is processed into active uroguanylin.

Two recently approved products in the US, linaclotide (SEQ ID NO:59) and plecanatide (SEQ ID NO:60) may be used as GCC agonists in the methods set forth herein.

Although proguanylin and prouroguanylin are precursors for mature guanylin and mature uroguanylin respectively, they may be used as GCC agonists as described herein provide they are delivered such that they can be processed into the mature peptides.

U.S. Pat. Nos. 5,140,102, 7,041,786 and 7,304,036, and U.S. Published Applications US 2004/0258687, US 2005/0287067, 20070010450, 20040266989, 20060281682, 20060258593, 20060094658, 20080025966, 20030073628, 20040121961 and 20040152868, which are each incorporated herein by reference, also refer to compounds which may bind to and activate guanylyl cyclase C.

In addition to human guanylin and human uroguanylin, guanylin or uroguanylin may be isolated or otherwise derived from other species such as cow, pig, goat, sheep, horse, rabbit, bison, etc. Such guanylin or uroguanylin may be administered to individuals including humans.

Antibodies including GCC binding antibody fragments can also be GCC agonists. Antibodies may include for example polyclonal and monoclonal antibodies including chimeric, primatized, humanized or human monoclonal antibodies as well as antibody fragments that bind to GCC with agonist activity such as CDRs, FAbs, F(Ab), Fv's including single chain Fv and the like. Antibodies may be IgE, IgA or IgM for example.

To reduce side effects caused by intestinal cell death, GCC agonists are delivered to the colorectal track by the oral delivery of such GCC agonists. ST peptides and the endogenous GCC agonist peptides, for example, are stable and can survive the stomach acid and pass through the small intestine to the colorectal track. Sufficient dosages are provided to ensure that GCC agonist reaches the large intestine in sufficient quantities to induce accumulation of cGM P in those cells as well.

GCC agonists such as for example ST, guanylin and uroguanylin, can survive the gastric environment. Thus, they may be administered without coating or protection against stomach acid. However, in order to more precisely control the release of GCC agonists administered orally, the GCC agonist may be enterically coated so that some or all of the GCC agonist is released after passing through the stomach. Such enteric coating may also be designed to provide a sustained or extended release of the GCC agonist over the period of time with which the coated GCC agonist passes through the intestines. In some embodiments, the GCC agonist may be formulated to ensure release of some compound upon entering the large intestine. In some embodiments, the GCC agonist may be delivered rectally.

Most enteric coatings are intended to protect contents from stomach acid. Accordingly, they are designed to release active agent upon passing through the stomach. The coatings and encapsulations used herein are provided to begin releasing the GCC agonist in the small intestine and preferably over an extended period of time so that GCC agonist concentrations can be maintained t an effective level for a greater period of time.

According to some embodiments, the GCC agonists are coated or encapsulated with a sufficient amount of coating material that the time required for the coating material to dissolve and release the GCC agonists corresponds with the time required for the coated or encapsulated composition to travel from the mouth to intestines.

According to some embodiments, the GCC agonists are coated or encapsulated with coating material that does not fully dissolve and release the GCC agonists until it comes in contact with conditions present in the small intestine. Such conditions may include the presence of enzymes in the colorectal track, pH, tonicity, or other conditions that vary relative to the stomach.

According to some embodiments, the GCC agonists are coated or encapsulated with coating material that is designed to dissolve in stages as it passes from stomach to small intestine to large intestine.

According to some embodiments, the GCC agonists are complexed with another molecular entity such that they are inactive until the GCC agonists cease to be complexed with molecular entity and are present in active form. In such embodiments, the GCC agonists are administered as “prodrugs” which become processed into active GCC agonists in the colorectal track.

Examples of technologies which may be used to formulate GCC agonists for sustained release when administered orally include, but are not limited to: U.S. Pat. Nos. 5,007,790, 4,451,260, 4,132,753, 5,407,686, 5,213,811, 4,777,033, 5,512,293, 5,047,248 and 5,885,616.

Examples of technologies which may be used to formulate GCC agonists or inducers for large intestine specific release when administered include, but are not limited to: U.S. Pat. No. 5,108,758 issued to Allwood, et al. on Apr. 28, 1992 which discloses delayed release formulations; U.S. Pat. No. 5,217,720 issued to Sekigawa, et al. on Jun. 8, 1993 which discloses coated solid medicament form having releasability in large intestine; U.S. Pat. No. 5,541,171 issued to Rhodes, et al. on Jul. 30, 1996 which discloses orally administrable pharmaceutical compositions; U.S. Pat. No. 5,688,776 issued to Bauer, et al. on Nov. 18, 1997 which discloses crosslinked polysaccharides, process for their preparation and their use; U.S. Pat. No. 5,846,525 issued to Maniar, et al. on Dec. 8, 1998 which discloses protected biopolymers for oral administration and methods of using same; U.S. Pat. No. 5,863,910 to Bolonick, et al. on Jan. 26, 1999 which discloses treatment of chronic inflammatory disorders of the gastrointestinal tract; U.S. Pat. No. 6,849,271 to Vaghefi, et al. on Feb. 1, 2005 which discloses microcapsule matrix microspheres, absorption-enhancing pharmaceutical compositions and methods; U.S. Pat. No. 6,972,132 to Kudo, et al. on Dec. 6, 2005 which discloses a system for release in lower digestive tract; U.S. Pat. No. 7,138,143 to Mukai, et al. Nov. 21, 2006 which discloses coated preparation soluble in the lower digestive tract; U.S. Pat. Nos. 6,309,666; 6,569,463, 6,214,378; 6,248,363; 6,458,383, 6,531,152, 5,576,020, 5,654,004, 5,294,448, 6,309,663, 5,525,634, 6,248,362, 5,843,479, and 5,614,220, which are each incorporated herein by reference.

In some embodiments, the effective amount is delivered so that sufficient accumulation of cGMP occurs. In some embodiments, the effective amount is delivered for at least a period of 2 hours. In some embodiments, the effective amount is present for up to 12 hours to several days. Multiple doses may be administered to maintain levels such that the amount of GCC agonist present, either free or bound to GCC, remains any or above the effective dose. In some embodiments, an initial loading dose and/or multiple administrations are required for cells of the intestine to become protected from radiation and chemotherapy induced cell death. After cells exposed to GCC agonist become resistant to cell death induced by radiation and chemotherapy, radiation or chemotherapeutics may be administered, in some cases in doses much higher than could be tolerated by patients who have not been pretreated with GCC agonist.

In some embodiments, GCC agonists which are peptides may be administered in an amount ranging from 100 ug to 1 gram every 4-48 hours. In some embodiments, GCC agonists are administered in an amount ranging from 1 mg to 750 mg every 4-48 hours. In some embodiments, GCC agonists are administered in an amount ranging from 10 mg to 500 mg every 4-48 hours. In some embodiments, GCC agonists are administered in an amount ranging from 50 mg to 250 mg every 4-48 hours. In some embodiments, GCC agonists are administered in an amount ranging from 75 mg to 150 mg every 4-48 hours,

In some embodiments, doses are administered every 4 or more hours. In some embodiments, doses are administered every 6 or more hours. In some embodiments, doses are administered every 8 or more hours. In some embodiments, doses are administered every 12 or more hours. In some embodiments, doses are administered every 24 or more hours. In some embodiments, doses are administered every 48 or more hours. In some embodiments, doses are administered every 4 hours or less. In some embodiments, doses are administered every 6 hours or less. In some embodiments, doses are administered every 8 hours or less. In some embodiments, doses are administered every 12 hours or less. In some embodiments, doses are administered every 24 hours or less. In some embodiments, doses are administered every 48 hours or less.

In some embodiments, additives or co-agents are administered in combination with GCC agonists to a minimize diarrhea or cramping/intestinal contractions-increased motility. For example, the individual may be administered a compound that before, simultaneously or after administration with a compound that relieves diarrhea. Such anti-diarrheal component may be incorporated in the formulation. Anti-diarrheal compounds and preparations, such as loperamide, bismuth subsalicylate and probiotic treatments such as strains of Lactobaccilus, are well known and widely available.

According to some aspects of the invention, innocuous bacteria of species that normally populate the colon are provided with genetic information needed to produce a guanylyl cyclase C agonist in the colon, making such guanylyl cyclase C agonist available to produce the effect of activating the guanylyl cyclase C on colon cells. The existence of a population of bacteria which can produce guanylyl cyclase C agonist provides a continuous administration of the guanylyl cyclase C agonist. In some embodiments, the nucleic acid sequences that encode the guanylyl cyclase C agonist may be under the control of an inducible promoter. Accordingly, the individual may turn expression on or off depending upon whether or not the inducer is ingested. In some embodiments, the inducer is formulated to be specifically released in the colon, thereby preventing induction of expression by the bacteria that may be populating other sites such as the small intestine. In some embodiments, the bacteria are is sensitive to a particular drug or auxotrophic such that it can be eliminated by administration of the drug or withholding an essential supplement.

The technology for introducing expressible forms of genes into bacteria is well known and the materials needed are widely available.

In some embodiments, bacteria which comprise coding sequences for a GCC agonist may be those of a species which commonly inhabits the intestinal track of an individual. Common gut flora include species from the genera Bacteroides, Clostridiun, Fusobacterium, Eubacterium, Ruminococcus, Peptococcus, Peptostreptococcus, Bifidobacteriu, Escherichia and Lactobacillus. In some embodiments, the bacteria selected is from a strain known to be useful as a probiotic. Examples of species of bacteria used as compositions for administration to humans include Bifdobacterium bifudum; Escherichia coli, Lactobacillus acidophilus, Lactobacillus rhammus, Lactobacillus casei, and Lactobaclus johnsonii. Other species include Lactobacillus hulgaricus, Streptococcus thermophilus, Bacillus coagdans and Lactobacillus bifidus. Examples of strains of bacteria used as compositions for administration to humans include: B. infantis 35624, (Align); Lactobacillus plantarum 299V; Bifdobacterium animalis DN-173 010; Bifidobacteriun animalis DN 173 010 (Activia Danone); Bfidobacterium animalis subsp. lactis BB-12 (Chr.Hansen); Bfidobacterium breve Yakult Bifiene Yakult; Bifidobacterium infntis 35624 Bipidobacterium lactis HN019 (DR 10) Howaru™ Bifido Danisco; Bifdobacterium longum BB536; Escherichia coli Nissle 1917; Lactobacillus acidophilus LA-5 Chr. Hansen; Lactobacillus acidophilus NCFM Rhodia Inc.; Lactobacillus casei DNI 14-001; Lactobacillus casei CRL431 Chr. Hansen; Lactobacillus casei F19 Cutura Arla Foods; Lactobacillus casei Shirota Yakult; Lactobacillus casei immunitass Actime1 Danone; Lactobacillus johnsonnii La1 (=Lactobacillus LC1) Nestlé; Lactobacillus plantarum 299V ProViva Probi IBS; Lactobacillus reuteri A TC 55730 BioGaia Biologics; Lactobacillus reuteri SD2112; Lactobacillus rhamnosus ATCC 53013 Vifit and others Valio; Lactobacillus rhamnosus LB21 Verum Norrmejerier; Lactobacillus salivarius UCC118; Lactococcus lactis L1A Verum Norrmejerier; Saccharomyces cerevisiae (boulardii) lyo; Streptococcus salivarius ssp thermophilus; Lactobacillus rhamnsus GR-1; Lactobacillus reuteri RC-14; Lactobacillus acidophilus CUL60; Bifidobacterium hifidum CUL 20; Lactobacillus helveticus R0052; and Lactobacillus rhamnosus R0011.

The following U.S. Patents, which are each incorporated herein by reference, disclose non-pathogenic bacteria which can be administered to individuals. U.S. Pat. Nos. 6,200,609; 6,524,574, 6,841,149, 6,878,373, 7,018,629, 7,101,565, 7,122,370, 7,172,777, 7,186,545, 7,192,581, 7,195,906, 7,229,818, and 7,244,424.

Accordingly, the aspects of the invention, bacteria would first be provided with genetic material encoding a GCC agonist in a form that would permit expression le of the agonist peptide within the bacteria, either constitutively or upon induction by the presence of an inducer that would turn on an inducible promoter.

Some embodiments comprise inducible regulatory elements such as inducible promoters. Typically, an inducible promoter is one in which an agent, when present, interacts with the promoter such that expression of the coding sequence operably linked to the promoter proceeds. Alternatively, an inducible promoter can include a repressor which is an agent that interacts with the promoter and prevent expression of the coding sequence operably linked to the promoter. Removal of the repressor results in expression of the coding sequence operably linked to the promoter.

The agents that induce an inducible promoter are preferably not naturally present in the organism where expression of the transgene is sought. Accordingly, the transgene is only expressed when the organism is affirmatively exposed to the inducing agent. Thus, in a bacterium that includes a transgene operably linked to an inducible promoter, when the bacterium is living within the gut of an individual, the promoter may be turned on and the transgene expressed when the individual ingests the inducing agent.

The agents that induce an inducible promoter are preferably not toxic. Thus, in a bacterium that includes a transgene operably linked to an inducible promoter, the inducing agent is preferably not toxic to the individual in whose gut the bacterium is living such that when the individual ingests the inducing agent to turn on expression of the transgene the inducing agent dose not have any severe toxic side effects on the individual.

The agents that induce an inducible promoter preferably affect only the expression of the gene of interest. Thus, in a bacterium that includes a transgene operably linked to an inducible promoter, the inducing agent does not have any significant effect on the expression of any other genes in the individual.

The agents that induce an inducible promoter preferably are easy to apply or removal. Thus, in a bacterium that includes a transgene operably linked to an inducible promoter that is living in the gut of an individual, the inducing agent is preferably an agent that can be easily delivered to the gut and that can be removed, either by affirmative neutralization for example or by metabolism/passing such that gene expression can be controlled

The agents that induce an inducible promoter preferably induce a clearly detectable expression pattern of either high or very low gene expression.

In some preferred embodiments, the chemically-regulated promoters are derived from organisms distant in evolution to the organisms where its action is required. Examples of inducible or chemically-regulated promoters include tetracycline-regulated promoters. Tetracycline-responsive promoter systems can function either to activate or repress gene expression system in the presence of tetracycline. Some of the elements of the systems include a tetracycline repressor protein (TetR), a tetracycline operator sequence (tetO) and a tetracycline transactivator fusion protein (tTA), which is the fusion of TetR and a herpes simplex virus protein 16 (VP16) activation sequence. The Tetracycline resistance operon is carried by the Escherichia coli transposon (Tn) 10. This operon has a negative mode of operation. The interaction between a repressor protein encoded by the operon, TetR, and a DNA sequence to which it binds, the tet operator (tetO), represses the activity of a promoter placed near the operator. In the absence of an inducer, TetR binds to tetO and prevents transcription. Transcription can be turned on when an inducer, such as tetracycline, binds to TetR and causes a conformation change that prevents TetR from remaining bound to the operator. When the operator site is not bound, the activity of the promoter is restored. Tetracycline, the antibiotic, has been used to create two beneficial enhancements to inducible promoters. One enhancement is an inducible on or off promoter. The investigators can choose to have the promoter always activated until Tet is added or always inactivated until Tet is added. This is the Tet on/off promoter. The second enhancement is the ability to regulate the strength of the promoter. The more Tet added, the stronger the effect.

Examples of inducible or chemically-regulated promoters include Steroid-regulated promoters. Steroid-responsive promoters are provided for the modulation of gene expression include promoters based on the rat glucocorticoid receptor (GR); human estrogen receptor (ER); ecdysone receptors derived from different moth species; and promoters from the steroid/retinoid/thyroid receptor superfamily. The hormone binding domain (HBD) of GR and other steroid receptors can also be used to regulate heterologous proteins in cis, that is, operatively linked to protein-encoding sequences upon which it acts. Thus, the HBD of GR, estrogen receptor (ER) and an insect ecdysone receptor have shown relatively tight control and high inducibility

Examples of inducible or chemically-regulated promoters include metal-regulated promoters. Promoters derived from metallothionein (proteins that bind and sequester metal ions) genes from yeast, mouse and human are examples of promoters in which the presence of metals induces gene expression.

IPTG is a classic example of a compound added to cells to activate a promoter. IPTG can be added to the cells to activate the downstream gene or removed to inactivate the gene.

U.S. Pat. No. 6,180,391, which is incorporated herein by reference, refers to a copper-inducible promoter.

U.S. Pat. No. 6,943,028, which is incorporated herein by reference, refers to highly efficient controlled expression of exogenous genes in E. coli.

U.S. Pat. No. 6,180,367, which is incorporated herein by reference, refers to a process for bacterial production of polypeptides.

Other examples of inducible promoters suitable for use with bacterial hosts include the beta.-lactamase and lactose promoter systems (Chang et al., Nature, 275: 615 (1978, which is incorporated herein by reference); Goeddel et al. Nature, 281: 544 (1979), which is incorporated herein by reference), the arabinose promoter system, including the araBAD promoter (Guzman et al., J. Bacteriol., 174: 7716-7728 (1992), which is incorporated herein by reference; Guzman et al., J. Bacteriol., 177: 4121-4130 (1995), which is incorporated herein by reference; Siegele and Hu, Proc. Natl. Acad. Sci. USA, 94: 8168-8172 (1997), which is incorporated herein by reference), the rhamnose promoter (Haldimann et al., J. Bacteriol., 180: 1277-1286 (1998), which is incorporated herein by reference), the alkaline phosphatase promoter, a tryptophan (trp) promoter system (Goeddel, Nucleic Acids Res., 8: 4057 (1980), which is incorporated herein by reference), the P.sub.LtetO-1 and P.sub.lac/are-1 promoters (Lutz and Bujard, Nucleic Acids Res., 25: 1203-1210 (1997), which is incorporated herein by reference), and hybrid promoters such as the tac promoter. deBoer et al., Proc. Natl. Acad. Sci. USA, 80: 21-25 (1983), which is incorporated herein by reference, However, other known bacterial inducible promoters and low-basal-expression promoters are suitable.

U.S. Pat. No. 6,083,715, which is incorporated herein by reference, refers to methods for producing heterologous disulfide bond-containing polypeptides in bacterial cells.

U.S. Pat. No. 5,830,720, which is incorporated herein by reference, refers to recombinant DNA and expression vector for the repressible and inducible expression of foreign genes.

U.S. Pat. No. 5,789,199, which is incorporated herein by reference, refers to a process for bacterial production of polypeptides.

U.S. Pat. No. 5,085,588, which is incorporated herein by reference, refers to bacterial promoters inducible by plant extracts.

U.S. Pat. No. 6,242,194, which is incorporated herein by reference, refers to probiotic bacteria host cells that contain a DNA of interest operably associated with a promoter of the invention can be orally administered to a subject . . . .

U.S. Pat. No. 5,364,780, which is incorporated herein by reference, refers to external regulation of gene expression by inducible promoters.

U.S. Pat. No. 5,639,635, which is incorporated herein by reference, refers to a process for bacterial production of polypeptides.

U.S. Pat. No. 5,789,199, which is incorporated herein by reference, refers to a process for bacterial production of polypeptides.

U.S. Pat. No. 5,689,044, which is incorporated herein by reference, refers to chemically inducible promoter of a plant PR-1 gene.

U.S. Pat. No. 5,063,154, which is incorporated herein by reference, refers to a pheromone-inducible yeast promoter.

U.S. Pat. No. 5,658,565, which is incorporated herein by reference, refers to an inducible nitric oxide synthase gene.

U.S. Pat. Nos. 5,589,392, 6,002,069, 5,693,531, 5,480,794, 6,171,816 6,541,224, 6,495,318, 5,498,538, 5,747,281, 6,635,482 and 5,364,780, which are each incorporated herein by reference, each refer to IPTG-inducible promoters.

U.S. Pat. Nos. 6,420,170, 5,654,168, 5,912,411, 5,891,718, 6,133,027, 5,739,018, 6,136,954, 6,258,595, 6,002,069 and 6,025,543, which are each incorporated herein by reference, each refer to tetracycline-inducible promoters.

### Guanylyl Cyclase A (GCA) Agonists (ANP, BNP)

Guanylyl cyclase-A/natriuretic peptide receptor-A (GCA) is a cellular protein involved in maintaining renal and cardiovascular homeostasis. GCA is a receptor found in kidney cells that binds to and is activated by two peptides made in the heart. Atrial natriuretic peptide (ANP, also referred to as cardiac atrial natriuretic peptide) is stored in the heart as pro-ANP and when released, is processed into mature ANP. B-type natriuretic peptide (BNP, also referred to as brain natriuretic peptide) is also produced in the heart. when ANP or BNP bounds to GCA, the GCA-expressing cells produce cGMP as a second messenger. Thus, ANP and BNP are GCA agonists which activate GCA and lead to accumulation of cGMP in cells expressing GCA.

ANP analogs that are GCA agonists are disclosed in Schiller P W, et al. Superactive analogs of the atrial natriuretic peptide (ANP), Biochem Biophys Res Commun. 1987 Mar. 13; 143(2):499-505; Schiller P W, et al. Synthesis and activity profiles of atrial natriuretic peptide (ANP) analogs with reduced ring size. Biochem Biophys Res Commun. 1986 Jul. 31; 138(2):880-6; Goghari M H, et al. Synthesis and biological activity profiles of atrial natriuretic factor (ANF) analogs., Int J Pept Protein Res. 1990 August; 36(2):156-60; Bovy P R, et al. A synthetic linear decapeptide binds to the atrial natriuretic peptide receptors and demonstrates cyclase activation and vasorelaxant activity. J Biol Chem. 1989 Dec. 5; 264(34):20309-13, and Schoenfeld et al. Molecular Pharmacology January 1995 vol. 47 no. 1 172-180.

### Guanylyl Cyclase B (GCB) Agonists (CNP)

Guanylyl cyclase B (GCB) is also referred to as natriuretic peptide receptor B, atrionatriuretic peptide receptor B and NPR2. GCB is the receptor for a small peptide (C-type natriuretic peptide) produced locally in many different tissues. GCA expression is reported in the kidney, ovarian cells, aorta, chondrocytes, the corpus cavernosum, the pineal gland among other.

While GCB is reported to bind to and be activated by ANP and BNP, C-type natriuretic peptide (CNP) is the most potent activator of GCB. ANP, BNP and CNP are GCB agonists. U.S. Pat. No. 5,434,133 and Furuya, M et al. Biochemical and Biophysical Research Communications, Volume 183, Issue 3, 31 Mar. 1992, Pages 964-%9, disclose CNP analogs.

### Soluble Guanylyl Cyclase Activators (Nitric Oxide, Nitrovasodilators, Protoprophyrin IX, and Direct Activators)

Soluble guanylyl cyclase (sGC) is heterodimeric protein made up of an alpha domain with C terminal region that has cyclase activity and a heme-binding beta domain which also has with a C terminal region that has cyclase activity. The sGC which is the only known receptor for nitric oxide has one heme per dimmer. The heme moiety in Fe(II) form is the target of NO. NO binding results in activation of sGC, i.e. a substantial increase in sGC activity. Activation of sGC is involved in vasodilation.

YC-1, which is 5-[1-(phenylmethyl)-1H-indazol-3-yl]-2-furanmethanol, is a nitric oxide (NO)-independent activator of soluble guanylyl cyclase. Ko F N et al. YC-1, a novel activator of platelet guanylate cyclase. Blood. 1994 Dec. 15; 84(12):4226-33.

Two drugs that activate sGC are cinaciguat (4-({(4-carboxybutyl)[2-(2-{[4-(2-phenylethyl) phenyl]methoxy}phenyl)ethyl]amino}methyl) benzoic acid) WO-0119780 7,087,644, 7,517,896 WO 20008003414 WO 2008148474 and riociguat, (Methyl N-[4,6-Diamino-2-[1-[(2-fluorophenyl)methyl]-1H-pyrazolo[3,4-b]pyridin-3-yl]-5-pyrimidinyl]-N-methyl-carbaminate) WO-03095451, which has been granted in the US as U.S. Ser. No. 07/173,037.

Other examples of sGC activators include 3-(5′-hydroxymethyl-2′-furyl)-1-benzylindazole (YC-1, Wu et al., Blood 84 (1994), 4226; Mulsch et al., Brit. J. Pharmacol. 120 (1997), 681), fatty acids (Goldberg et al, J. Biol. Chem. 252(1977), 1279), diphenyliodonium hexafluorophosphate (Pettibone et al., Eur. J. Pharmacol. 116 (1985), 307), isoliquiritigenin (Yu et. al., Brit. J. Pharmacol. 114 (1995), 1587) and various substituted pyrazole derivatives (WO 98/16223). In addition, WO 98/16507, WO 98/23619, WO 00/06567, WO 00/06568, WO 00/06569, WO 00/21954 WO 02/42299, WO 02/42300, WO 02/42301, WO 02/42302, WO 02/092596 and WO 03/004503 describe pyrazolopyridine derivatives as stimulators of soluble guanylate cyclase. Also described inter alia therein are pyrazolopyridines having a pyrimidine residue in position 3. Compounds of this type have very high in vitro activity in relation to stimulating soluble guanylate cyclase. However, it has emerged that these compounds have disadvantages in respect of their in vivo properties such as, for example, their behavior in the liver, their pharmacokinetic behavior, their dose-response relation or their metabolic pathway.

Other sGC activators are disclosed in O. V. Evgenov et al. Nature Rev. Drug Disc. 5 (2006), 755; and US Published Patent Application Publication Nos. 20110034450, 20100210643, 20100197680, 20100168240, 20100144864, 20100144675, 20090291993, 20090286882, 20090215843, 20080

### PDE Inhibitors

In some embodiments, the active agent comprises PDE inhibitors including, for example, nonselective phosphodiesterase inhibitors, PDE1 selective inhibitors, PDE2 selective inhibitors, PDE3 selective inhibitors, PDE4 selective inhibitors, PDE5 selective inhibitors, and PDE10 selective inhibitors.

PDE inhibitors are generally discussed in the following references which are each incorporated herein by reference: Uzunov, P. and Weiss, B.: Separation of multiple molecular forms of cyclic adenosine 3′,5′-monophosphate phosphodiesterase in rat cerebellum by polyacrylamide gel electrophoresis. Biochim. Biophys. Acta 284:220-226, 1972; Weiss, B.: Differential activation and inhibition of the multiple forms of cyclic nucleotide phosphodiesterase. Adv. Cycl. Nucl. Res. 5:195-211, 1975; Fertel, R. and Weiss, B.: Properties and drug responsiveness of cyclic nucleotide phosphodiesterases of rat lung. Mol. Pharmacol. 12:678-687, 1976; Weiss, B. and Hait, W. N.: Selective cyclic nucleotide phosphodiesterase inhibitors as potential therapeutic agents. Ann. Rev. Pharmacol. Toxicol. 17:441-477, 1977; Essayan D M. (2001). “Cyclic nucleotide phosphodiesterases.”. J Allergy Clin Immunol. 108 (5): 671-80; Deree J, Martins J O, Melbostad H, Loomis W H, Coimbra R. (2008). “Insights into the Regulation of TNF-α Production in Human Mononuclear Cells: The Effects of Non-Specific Phosphodiesterase Inhibition”. Clinics (Sao Paulo). 63 (3): 321-8; Marques L J, Zheng L, Poulakis N, Guzman J, Costabel U (February 1999). “Pentoxifylline inhibits TNF-alpha production from human alveolar macrophages”. Am. J. Respir. Crit. Care Med. 159 (2): 508-11; Peters-Golden M, Canetti C, Mancuso P, Coffey M J. (2005). “Leukotrienes: underappreciated mediators of innate immune responses”. J Immunol. 174 (2): 589.94; Daly J W, Jacobson K A, Ukena D. (1987). “Adenosine receptors: development of selective agonists and antagonists”. Prog Clin Biol Res. 230 (1): 41-63; MacCorquodale D W. THE SYNTHESIS OF SOME ALKYLXANTHINES. Journal of the American Chemical Society. 1929 July; 51(7):2245-2251; WO/1985/002540; U.S. Pat. No. 4,288,433; Daly J W, Padgett W L, Shamim M T (July 1986). “Analogues of caffeine and theophylline: effect of structural alterations on affinity at adenosine receptors”. Journal of Medicinal Chemistry 29 (7): 1305-8; Daly J W, Jacobson K A, Ukena D (1987). “Adenosine receptors: development of selective agonists and antagonists”. Progress in Clinical and Biological Research 230:41-63; Choi O H, Shamim M T, Padgett W L, Daly J W (1988). “Caffeine and theophylline analogues: correlation of behavioral effects with activity as adenosine receptor antagonists and as phosphodiesterase inhibitors”. Life Sciences 43 (5): 387-98; Shamim M T, Ukena D, Padgett W L, Daly J W (June 1989). “Effects of 8-phenyl and 8-cycloalkyl substituents on the activity of mono-, di-, and trisubstituted alkylxanthines with substitution at the 1-, 3-, and 7-positions”. Journal of Medicinal Chemistry 32 (6): 1231-7; Daly J W, Hide I, Müller C E, Shamim M (1991). “Caffeine analogs: structure-activity relationships at adenosine receptors”. Pharmacology 42 (6): 309-21; Ukena D, Schudt C, Sybrecht G W (February 1993). “Adenosine receptor-blocking xanthines as inhibitors of phosphodiesterase isozymes”. Biochemical Pharmacology 45 (4): 847-51. doi:10.1016/0006-2952(93)9168-V; Daly J W (July 2000). “Alkylxanthines as research tools”. Journal of the Autonomic Nervous System 81 (1-3): 44-52. doi:10.1016S0165-1838(00)00110-7; Daly J W (August 2007). “Caffeine analogs: biomedical impact”. Cellular and Molecular Life Sciences: CMLS 64 (16): 2153-69; González M P, Terán C, Teijeira M (May 2008). “Search for new antagonist ligands for adenosine receptors from QSAR point of view. How close are we?”. Medicinal Research Reviews 28 (3): 329-71; Baraldi P G, Tabrizi M A, Gessi S, Borea P A (January 2008). “Adenosine receptor antagonists: translating medicinal chemistry and pharmacology into clinical utility”. Chemical Reviews 108 (1): 238-63; de Visser Y P, Walther F J, Laghmani E H, van Wijngaarden S, Nieuwland K, Wagenaar G T. (2008). “Phosphodiesterase-4 inhibition attenuates pulmonary inflammation in neonatal lung injury”. Eur Respir J 31 (3): 633-644; Yu M C, Chen J H, Lai C Y, Han C Y, Ko W C. (2009). “Luteolin, a non-selective competitive inhibitor of phosphodiesterases 1-5, displaced [(3)H]-rolipram from high-affinity rolipram binding sites and reversed xylazine/ketamine-induced anesthesia”. Eur J Pharmacol. 627 (1-3):269-75; Bobon D, Breulet M, Gerard-Vandenhove M A, Guiot-Goffioul F, Plomtcux G, Sastre-y-Hernandez M, Schratzer M, Troisfontaines B, von Frenckell R, Wachtel H. (1988). “Is phosphodiesterase inhibition a new mechanism of antidepressant action? A double-blind double-dummy study between rolipram and desipramine in hospitalized major and/or endogenous depressives”. Eur Arch Psychiatry Neurol Sci. 238 (1): 2-6; Maxwell C R, Kanes S J, Abel T, Siegel S J. (2004). “Phosphodiesterse inhibitors: a novel mechanism for receptor-independent antipsychotic medications”. Neuroscience. 129 (1): 101-7; Kanes S J, Tokarczyk J, Siegel S J, Bilker W, Abel T, Kelly M P. (2006). “Rolipram: A specific phosphodiesterase 4 inhibitor with potential antipsychotic activity”. Neuroscience. 144 (1): 239-46; and Vecsey C G, Baillie G S, Jaganath D, Havekes R, Daniels A, Wimmer M, Huang T, Brown K M. Li X Y, Descalzi G, Kim S S, Chen T, Shang Y Z, Zhuo M, Houslay M D, Abel T. (2009). “Sleep deprivation impairs cAMP signaling in the hippocampus”. Nature. 461 (7267): 1122-1125.

In addition to activation of guanylyl cyclases, cGMP levels can be elevated and cells protected from chemotherapeutics and radiation therapy using PDE such as PDE 1, PDE2, PDE3, PDE4, PDE5 and PDE10 inhibitors. The breakdown of cGMP is controlled by a family of phosphodiesterase (PDE) isoenzymes. To date, seven members of the family have been described (PDE I-VII) the distribution of which varies from tissue to tissue (Beavo & Reifsnyder (1990) TIPS, 11:150-155 and Nicholson et al (1991) TIPS, 12: 19-27). Specific inhibitors of PDE isoenzymes may be useful to achieve differential elevation of cGMP in different tissues. Some PDE inhibitors specifically inhibit breakdown of cGMP while not effecting cAMP. In some embodiments, possible PDE inhibitors may be PDE3 inhibitors, PDE4 inhibitors, PDE5 inhibitors, PDE3/4 inhibitors or PDE3/4/5 inhibitors.

PDE inhibitors which elevate cGMP specifically are disclosed in U.S. Pat. Nos. 6,576,644, 7,384,958, 7,276,504, 7,273,868, 7,220,736, 7,098,209, 7,087,597, 7,060,721, 6,984,641, 6,930,108, 6,911,469, 6,784,179, 6,656,945, 6,642,244, 6,476,021, 6,326,379, 6,316,438, 6,306,870, 6,300,335, 6,218,392, 6,197,768, 6,037,119, 6,025,494, 6,018,046, 5,869,516, 5,869,486, 5,716,993. Other examples include compounds disclosed in WO 96/05176 and 6,087,368, U.S. Pat. Nos. 4,101,548, 4,001,238, 4,001,237, 3,920,636, 4,060,615, 4,209,623, 5,354,571, 3,031,450, 3,322,755, 5,401,774, 5,147,875, 4,885,301, 4,162,316, 4,047,404, 5,614,530, 5,488,055, 4,880,810, 5,439,895, 5,614,627, GB 2 063 249, EP 0 607 439, WO 97/03985, EP 0 395 328, EP 0 428 268, PCT WO 93/12095, WO 93/07149, EP 0 349 239, EP 0 352 960, EP 0 526 004, EP 0 463 756, EP 0 607 439, WO 94/05661, EP 0 351058, EP 0 347 146, WO 97/03985, WO 97/03675, WO 95/19978, WO 98/08848, WO 98/16521, EP 0 722 943, EP 0 722 937, EP 0 722 944, WO 98/17668, WO 97/24334, WO 98/06722, PCT/JP97/03592, WO 98/23597, WO 94/29277, WO 98/14448, WO 97/03070, WO 98/38168, WO 96/32379, and PCT/GB98/03712. PDE inhibitors may include those disclosed in the following patent applications and patents: DE1470341, DE2108438, DE2123328, DE2305339, DE2305575, DE2315801, DE2402908, DE2413935, DE2451417, DE2459090, DE2646469, DE2727481, DE2825048, DE2837161, DE2845220, DE2847621, DE2934747, DE3021792, DE3038166, DE3044568, EP000718, EP0008408, EP0010759, EP0059948, EP0075436, EP0096517, EP0112987, EP0116948, EP0150937, EP0158380, EP0161632, EP0161918, EP0167121, EP0199127, EP0220044, EP0247725, EP0258191, EP0272910, EP0272914, EP0294647, EP0300726, EP0335386, EP0357788, EP0389282, EP0406958, EP0426180, EP0428302, EP0435811, EP0470805, EP0482208, EP0490823, EP0506194, EP0511865, EP0527117, EP0626939, EP0664289, EP0671389, EP0685474, EP0685475, EP0685479, JP92234389, JP94329652, JP95010875, U.S. Pat. Nos. 4,963,561, 5,141,931, WO9117991, WO9200968, WO9212961, WO9307146, WO9315044, WO9315045, WO9318024, WO9319068, WO9319720, WO9319747, WO9319749, WO9319751, WO9325517, WO9402465, WO9406423, WO9412461, WO9420455, WO9422852, WO9425437, WO9427947, WO9500516, WO9501980, WO9503794, WO9504045, WO9504046, WO9505386, WO9508534, WO9509623, WO9509624, WO9509627, WO9509836, WO9514667, WO9514680, WO9514681, WO9517392, WO9517399, WO9519362, WO9522520, WO9524381, WO9527692, WO9528926, WO9535281, WO9535282, WO9600218, WO9601825, WO9602541, WO9611917, DE3142982, DE1116676, DE2162096, EP0293063, EP0463756, EP0482208, EP0579496, EP0667345 and WO9307124, EP0163965, EP0393500, EP0510562, EP0553174, WO9501338 and WO9603399.

Examples of nonselective phosphodiesterase inhibitors include: methylated xanthines and derivatives such as for examples: caffeine, a minor stimulant, aminophylline, IBMX (3-isobutyl-1-methylxanthine), used as investigative tool in pharmacological research, paraxanthine, pentoxifylline, a drug that has the potential to enhance circulation and may have applicability in treatment of diabetes, fibrotic disorders, peripheral nerve damage, and microvascular injuries, theobromine and theophylline, a bronchodilator. Methylated xanthines act as both competitive nonselective phosphodiesterase inhibitors which raise intracellular cAMP, activate PKA, inhibit TNF-alpha and leukotriene synthesis, and reduce inflammation and innate immunity and nonselective adenosine receptor antagonists. Different analogues show varying potency at the numerous subtypes, and a wide range of synthetic xanthine derivatives (some nonmethylated) have been developed in the search for compounds with greater selectivity for phosphodiesterase enzyme or adenosine receptor subtypes.

PDE inhibitors include 1-(3-Chlorophenylamino)-4-phenylphthalazine and dipyridamol. Another PDE1 selective inhibitor is, for example, Vinpocetine.

PDE2 selective inhibitors include for example, EHNA (erythro-9-(2-hydroxy-3-nonyl)adenine) and Anagrelide.

PDE3 selective inhibitors include for example, sulmazoe, ampozone, ciostamide, carbazeran piroximone, imazodan, siguazodan, adibendan, saterinone, emoradan, revizinone, and enoximone and milrinone. Some are used clinically for short-term treatment of cardiac failure. These drugs mimic sympathetic stimulation and increase cardiac output. PDE3 is sometimes referred to as cGMP-inhibited phosphodiesterase.

Examples of PDE3/4 inhibitors include benafentrine, trequinsin, zardaverine and tolafentrine.

PDE4 selective inhibitors include for example: winlcuder, denbufylline, rolipram, oxagrelate, nirtaquazone, motapizone, lixazinone, indolidan, olprinone, atizoram, dipamfylline, arofylline, filaminast, piclamilast, tibenelast, mopidamol, anagrelide, ibudilast, amrinone, pimobendan, cilostazol, quazinone and N-(3,5-dichloropyrid-4-yl)-3-cycopropylmethoxy4-difluoromethoxybenzamide. Mesembrine, an alkaloid from the herb Sceletium tortuosum; Rolipram, used as investigative tool in pharmacological research; Ibudilast, a neuroprotective and bronchodilator drug used mainly in the treatment of asthma and stroke (inhibits PDE4 to the greatest extent, but also shows significant inhibition of other PDE subtypes, and so acts as a selective PDE4 inhibitor or a non-selective phosphodiesterase inhibitor, depending on the dose); Piclamilast, a more potent inhibitor than rolipram; Luteolin, supplement extracted from peanuts that also possesses IGF-1 properties; Drotaverine, used to alleviate renal colic pain, also to hasten cervical dilatation in labor, and Roflumilast, indicated for people with severe COPD to prevent symptoms such as coughing and excess mucus from worsening. PDE4 is the major cAMP-metabolizing enzyme found in inflammatory and immune cells. PDE4 inhibitors have proven potential as anti-inflammatory drugs, especially in inflammatory pulmonary diseases such as asthma, COPD, and rhinitis. They suppress the release of cytokines and other inflammatory signals, and inhibit the production of reactive oxygen species. PDE4 inhibitors may have antidepressive effects [26] and have also recently been proposed for use as antipsychotics.

PDE5 selective inhibitors include for example: Sildenafil, tadalafil, vardenafil, vesnarinone, zaprinast lodenafil, mirodenafil, udenafil and avanafil. PDE5, is cGMP-specific is responsible for the degradation of cGMP in the corpus cavernosum (these phosphodiesterase inhibitors are used primarily as remedies for erectile dysfunction, as well as having some other medical applications such as treatment of pulmonary hypertension); Dipyridamole (results in added benefit when given together with NO or statins); and newer and more-selective inhibitors are such as icariin, an active component of Epimedium grandiflorum, and possibly 4-Methylpiperazine and Pyrazolo Pyrimidin-7-1, components of the lichen Xanthoparmelia scabrosa.

PDE10 is selective inhibited by Papaverine, an opium alkaloid. PDE10A is almost exclusively expressed in the striatum and subsequent increase in cAMP and cGMP after PDE10A inhibition (e.g. by papaverine) is “a novel therapeutic avenue in the discovery of antipsychotics”.

Additional PDE inhibitors include those set forth in U.S. Pat. Nos. 8,153,104, 8,133,903, 8,114,419, 8,106,061, 8,084,261, 7,951,397, 7,897,633, 7,807,803, 7,795,378, 7,750,015, 7,737,155, 7,732,162, 7,723,342, 7,718,702, 7,671,070, 7,659,273, 7,605,138, 7,585,847, 7,576,066, 7,569,553, 7,563,790, 7,470,687, 7,396,814, 7,393,825, 7,375,100, 7,363,076, 7,304,086, 7,235,625, 7,153,824, 7,091,207, 7,056,936, 7,037,257, 7,022,709, 7,019,010, 6,992,070, 6,969,719, 6,964,780, 6,875,575, 6,743,799, 6,740,306, 6,716,830, 6,670,394, 6,642,244, 6,610,652, 6,555,547, 6,548,508, 6,541,487, 6,538,005, 6,534,519, 6,534,518, 6,479,505, 6,476,025, 6,436,971, 6,436,944, 6,428,478, 6,423,683, 6,399,579, 6,391,869, 6,380,196, 6,376,485, 6,333,354, 6,306,869, 6,303,789, 6,294,564, 6,288,118, 6,271,228, 6,235,782, 6,235,776, 6,225,315, 6,177,471, 6,143,757, 6,143,746, 6,127,378, 6,103,718, 6,080,790, 6,080,782, 6,077,854, 6,066,649, 6,060,501, 6,043,252, 6,011,037, 5,998,428, 5,962,492, 5,922,557, 5,902,824, 5,891,896, 5,874,437, 5,871,780, 5,866,593, 5,859,034, 5,849,770, 5,798,373, 5,786,354, 5,776,958, 5,712,298, 5,693,659, 5,681,961, 5,674,880, 5,622,977, 5,580,888, 5,491,147, 5,426,119, and 5,294,626, which are each incorporated herein by reference. Additional PDE2 inhibitors include those set forth in U.S. Pat. Nos. 6,555,547, 6,538,029, 6,479,493 and 6,465,494, which are each incorporated herein by reference. Additional PDE3 inhibitors include those set forth in U.S. Pat. Nos. 7,375,100, 7,056,936, 6,897,229, 6,716,871, 6,498,173, and 6,110,471, which are each incorporated herein by reference. Additional PDE4 inhibitors include those set forth in U.S. Pat. Nos. 8,153,646, 8,110,682, 8,030,340, 7,964,615, 7,960,433, 7,951,954, 7,902,224, 7,846,973, 7,759,353, 7,659,273, 7,557,247, 7,550,475, 7,550,464, 7,538,127, 7,517,889, 7,446,129, 7,439,393, 7,402,673, 7,375,100, 7,361,787, 7,253,189, 7,135,600, 7,101,866, 7,060,712, 7,056,936, 7,045,658, 6,953,774, 6,884,802, 6,858,596, 6,787,532, 6,747,043, 6,740,655, 6,713,509, 6,630,483, 6,436,971, 6,288,118, and 5,919,801, which are each incorporated herein by reference. Additional PDE5 inhibitors include those set forth in U.S. Pat. Nos. 7,449,462, 7,375,100, 6,969,507, 6,723,719, 6,677,335, 6,660,756, 6,538,029, 6,479,493, 6,476,078, 6,465,494, 6,451,807, 6,143,757, 6,143,746 and 6,043,252, which are each incorporated herein by reference. Additional PDE10 inhibitors include those set forth in U.S. Pat. No. 6,538,029 which is incorporated herein by reference.

### MRP Inhibitors

The human multidrug resistance proteins MRP4 and MRP5 are organic anion transporters that have the unusual ability to transport cyclic nucleotides including cGM P. Accordingly, cGMP levels may be increased by inhibition of MRP4 and MRP5. Compounds that inhibit MRP4 and MRP5 may include dipyridamole, dilazep, nitrobenzyl mercaptopurine riboside, sildenafil, trequinsin, zaprinast and MK571 (3-[[[3-[(1E)-2-(7-Chloro-2-quinolinyl)ethenyl]phenyl][[3-(dimethylamino)-3-oxopropyl]thio]methylthio]propanoic acid). These compounds may be more effective at inhibiting MRP4 than MRP5. Other compounds which may be useful as MRP inhibitors include sulfinpyrazone, zidovudine-monophosphate, genistein, indomethacin, and probenecid.

Cyclic GMP and/or cGMP Analogues

In some embodiments, the active agent comprises cyclic GMP. In some embodiments, the active agent comprises cGMP analogues such as for example 8-bromo-cGMP and 2-chloro-cGMP.

### Controlled Release Formulations

Controlled release compositions are provided for delivering to tissues of the duodenum, small intestine, large intestine, colon and/or rectum. The controlled release formulations comprise one or more active agents selected from the group consisting of: Guanylyl cyclase A (GCA) agonists (ANP, BNP), Guanylyl cyclase B(GCB) agonists (CNP), Soluble guanylyl cyclase activators (nitric oxide, nitrovasodilators, protoprophyrin IX, and direct activators), Guanylyl cyclase C agonists, PDE Inhibitors, MRP inhibitors, cyclic GMP and cGMP analogues, wherein the active agents are formulated as a controlled release composition for controlled release to tissues of the duodenum, small intestine, large intestine, colon and/or rectum. Method of preventing GI syndrome in an individual undergoing chemotherapy or radiation therapy to treat cancer are provided which comprise the step of, prior to administration of chemotherapy or radiation to the individual, administering to the individual by oral administration an amount of the controlled release composition sufficient to elevate intracellular cGMP levels in gastrointestinal cells sufficient to arrest cell proliferation of said gastrointestinal cells and/or maintain genomic integrity by enhanced DNA damage sensing and repair for a period sufficient to prevent GI syndrome. Methods of reducing gastrointestinal side effects in an individual undergoing chemotherapy or radiation therapy to treat cancer are provided which comprise the step of, prior to administration of chemotherapy or radiation to the individual, administering to the individual by oral administration an amount of the controlled release composition sufficient to elevate intracellular cGMP levels in gastrointestinal cells sufficient to arrest cell proliferation of said gastrointestinal cells and/or maintain genomic integrity by enhanced DNA damage sensing and repair for a period sufficient to increase survival of gastrointestinal cells and reduce severity of chemotherapy or radiation therapy side effects. Methods of treating an individual who has cancer are provided that comprise the steps of administering by oral administration to the individual the controlled release composition in an amount that elevates intracellular cGMP levels in gastrointestinal cells sufficient to arrest cell proliferation of said gastrointestinal cells and/or maintain genomic integrity by enhanced DNA damage sensing and repair for a period sufficient to prevent GI syndrome; and administering to said individual chemotherapy or radiation an amount sufficient to treat cancer. Methods of treating an individual who has cancer are provided that comprise the steps of administering by oral administration to the individual the controlled release composition in an amount that elevates intracellular cGMP levels in gastrointestinal cells sufficient to arrest cell proliferation of said gastrointestinal cells and/or maintain genomic integrity by enhanced DNA damage sensing and repair for a period sufficient to increase survival of gastrointestinal cells and reduce severity of chemotherapy or radiation therapy side effects; and administering to said individual chemotherapy or radiation an amount sufficient to treat cancer. Methods of preventing GI syndrome in an individual who has been exposed to or who is at risk of exposure to sufficient doses of radiation to cause GI syndrome are provided that comprise the step of administering by oral administration to the individual who has been exposed to or who is at risk of exposure to sufficient doses of radiation to cause GI syndrome, an amount of the controlled release composition that elevates intracellular cGMP levels in gastrointestinal cells sufficient to prevent GI syndrome. Methods of treating an individual who has been exposed to a sufficient amount of radiation to cause radiation sickness are provided that comprise the step of administering to said individual by oral administration, an amount of the controlled release composition that elevates cGMP levels in gastrointestinal cells sufficient to elevate intracellular cGMP levels in gastrointestinal cells sufficient to arrest cell proliferation of said gastrointestinal cells and/or maintain genomic integrity by enhanced DNA damage sensing and repair for a period sufficient to reduce gastrointestinal damage. Methods of preventing side effects in an individual who is undergoing chemotherapy or radiation are provided that comprise the steps of administering to said individual by oral administration prior to administration of chemotherapy or radiation the controlled release composition that elevates cGMP levels in cells to be protected sufficient to arrest cell proliferation of said cells and/or maintain genomic integrity by enhanced DNA damage sensing and repair for a period sufficient to reduce damage to said cells. Methods of treating an individual who has cancer are provided that comprise the steps of administering to said individual an amount of the controlled release composition that elevates cGMP levels in cells to be protected sufficient to arrest cell proliferation of said cells and/or maintain genomic integrity by enhanced DNA damage sensing and repair for a period sufficient to reduce damage to said cells; and administering to said individual chemotherapy or radiation an amount sufficient to treat cancer.

In some embodiments, methods comprise delivery of one or more active agents selected from the group consisting of: Guanylyl cyclase A (GCA) agonists (ANP, BNP), Guanylyl cyclase B (GCB) agonists (CNP), Guanylyl cyclase C (GCC) agonists, Soluble guanylyl cyclase activators (nitric oxide, nitrovasodilators, protoprophyrin IX, and direct activators), PDE Inhibitors, MRP inhibitors, cyclic GMP and cGMP analogues wherein the active agents are formulated for controlled release such that the release of the at least some if not the majority or all of the active agent bypasses the stomach and is delivered to tissues of the duodenum, small intestine, large intestine, colon and/or rectum. These formulations are particularly useful in those cases in which the active agent is either inactivated by the stomach or taken up by the stomach, in either case thereby preventing the active agent from reaching the tissue downstream of the stomach where activity is desirable. In some embodiments, the preferred site of release the duodenum. In some embodiments, the preferred site of release the small intestine. In some embodiments, the preferred site of release the large intestine. In some embodiments, the preferred site of release the colon. Bypassing the stomach and releasing the drug after it has passed through the stomach ensures tissue specific delivery of active agent in effective amounts.

The methods provide more effective delivery of active agents to colorectal track including the duodenum, the small and large intestines and the colon. Formulations are provided to deliver active agent throughout the colorectal track or to specific tissue within in.

Some embodiments utilize GCC Agonists, Guanylyl cyclase A (GCA) agonists (ANP, BNP), Guanylyl cyclase B (GCB) agonists (CNP), Soluble guanylyl cyclase activators (nitric oxide, nitrovasodilators, protoprophyrin IX, and direct activators), PDE Inhibitors, MRP inhibitors and/or cyclic GMP and/or cGMP analogues and/or PDE inhibitors formulated from controlled release whereby the release of the at least some if not the majority or all of the active agent bypasses the stomach and is delivered to tissues of the duodenum, small intestine, large intestine, colon and/or rectum. These formulations are particularly useful in those cases in which the active agent is either inactivated by the stomach or taken up by the stomach, in either case thereby preventing the active agent from reaching the tissue downstream of the stomach where activity is desirable. In some embodiments, the preferred site of release the duodenum. In some embodiments, the preferred site of release the small intestine. In some embodiments, the preferred site of release the large intestine. In some embodiments, the preferred site of release the colon.

Most enteric coatings are intended to protect contents from stomach acid. Accordingly, they are designed to release active agent upon passing through the stomach. The coatings and encapsulations used herein are provided to release active agents upon passing the colorectal track. This can be accomplished in several ways.

Enteric formulations are described in U.S. Pat. Nos. 4,601,896, 4,729,893, 4,849,227, 5,271,961, 5,350,741, and 5,399,347. Oral and rectal formulations are taught in Remington's Pharmaceutical Sciences, 18th Edition, 1990, Mack Publishing Co., Easton Pa. which is incorporated herein by reference.

According to some embodiments, active agents are coated or encapsulated with a sufficient amount of coating material that the time required for the coating material to dissolve and release the active agents corresponds with the time required for the coated or encapsulated composition to travel from the mouth to the colorectal track.

According to some embodiments, the active agents are coated or encapsulated with coating material that does not fully dissolve and release the active agents until it comes in contact with conditions present in the colorectal track. Such conditions may include the presence of enzymes in the colorectal track, pH, tonicity, or other conditions that vary relative to the small intestine.

According to some embodiments, the active agents are coated or encapsulated with coating material that is designed to dissolve in stages as it passes from stomach to small intestine to large intestine. The active agents are released upon dissolution of the final stage which occurs in the colorectal track.

In some embodiments, the formulations are provided for release of active agent in specific tissues or regions of the colorectal track, for example, the duodenum, the small intestine, the large intestine or the colon.

Examples of technologies which may be used to formulate active agents for large intestine specific release when administered include, but are not limited to: U.S. Pat. No. 5,108,758 issued to Allwood, et al. on Apr. 28, 1992 which discloses delayed release formulations; U.S. Pat. No. 5,217,720 issued to Sekigawa, et al. on Jun. 8, 1993 which discloses coated solid medicament form having releasability in large intestine; U.S. Pat. No. 5,541,171 issued to Rhodes, et al. on Jul. 30, 1996 which discloses orally administrable pharmaceutical compositions; U.S. Pat. No. 5,688,776 issued to Bauer, et al. on Nov. 18, 1997 which discloses crosslinked polysaccharides, process for their preparation and their use; U.S. Pat. No. 5,846,525 issued to Maniar, et al. on Dec. 8, 1998 which discloses protected biopolymers for oral administration and methods of using same; U.S. Pat. No. 5,863,910 to Bolonick, et al. on Jan. 26, 1999 which discloses treatment of chronic inflammatory disorders of the gastrointestinal tract; U.S. Pat. No. 6,849,271 to Vaghefi, et al. on Feb. 1, 2005 which discloses microcapsule matrix microspheres, absorption-enhancing pharmaceutical compositions and methods; U.S. Pat. No. 6,972,132 to Kudo, et al. on Dec. 6, 2005 which discloses a system for release in lower digestive tract; U.S. Pat. No. 7,138,143 to Mukai, et al. Nov. 21, 2006 which discloses coated preparation soluble in the lower digestive tract; U.S. Pat. Nos. 6,309,666; 6,569,463, 6,214,378; 6,248,363; 6,458,383, 6,531,152, 5,576,020, 5,654,004, 5,294,448, 6,309,663, 5,525,634, 6,248,362, 5,843,479, and 5,614,220, which are each incorporated herein by reference.

Controlled release formulations are well known including those which are particularly suited for release of active agent into the duodenum. Examples of controlled release formulations which may be used include U.S. Patent Application Publication 2010/0278912, U.S. Pat. No. 4,792,452, U.S. Patent Application Publication 2005/0080137, U.S. Patent Application Publication 200610159760, U.S. Patent Application Publication 2011/0251231, U.S. Pat. No. 5,443,843, U.S. Patent Application Publication 2008/0153779, U.S. Patent Application Publication 2009/0191282, U.S. Patent Application Publication 2003/0228362, U.S. Patent Application Publication 2004/0224019, U.S. Patent Application Publication 2010/0129442, U.S. Patent Application Publication 2007/0148153, U.S. Pat. Nos. 5,536,507, 7,790,755, U.S. Patent Application Publication 2005/0058704, U.S. Patent Application Publication 2001/0026800, U.S. Patent Application Publication 2009/0175939, US 2002/0192285, U.S. Patent Application Publication 2008/0145417, U.S. Patent Application Publication 2009/0053308, U.S. Pat. No. 8,043,630, U.S. Patent Application Publication 2011/0053866, U.S. Patent Application Publication 2009/0142378, U.S. Patent Application Publication 2006/0099256, U.S. Patent Application Publication 2009/0104264, U.S. Patent Application Publication 2004/0052846, U.S. Patent Application Publication 2004/0053817, U.S. Pat. Nos. 4,013,784, 5,693,340, U.S. Patent Application Publication 2011/0159093, U.S. Patent Application Publication 2009/0214640, U.S. Pat. Nos. 5,133,974, 5,026,559, U.S. Patent Application Publication 2010/0166864, U.S. Patent Application Publication 2002/0110595, U.S. Patent Application Publication 2007/0148153, U.S. Patent Application Publication 2009/0220611, U.S. Patent Application Publication 2010/0255087 and U.S. Patent Application Publication 2009/0042889, each of which is incorporated herein by reference. Other examples of technologies which may be used to formulate active agents for sustained release when administered orally include, but are not limited to: U.S. Pat. Nos. 5,007,790, 4,451,260, 4,132,753, 5,407,686, 5,213,811, 4,777,033, 5,512,293, 5,047,248 and 5,885,616.

### Patient Populations

Prior to receiving anticancer chemotherapy or radiation, patients undergoing chemotherapy and/or radiation therapy may be provided with compositions which elevate cGMP levels in non-cancer tissues that comprise dividing cells such as gastrointestinal tissue in order to protect those tissues from deleterious side effects brought on by non-specific toxicity against dividing cells. Elevated levels of cGMP are maintained during the period of time chemotherapeutics and/or radiation is a present. By elevating cGMP levels in non-cancer cells, individual patients will experience reduced toxicity and side effects which often accompany chemotherapy and radiation. Higher doses of chemotherapy and radiation may be tolerated because of reduced side effects to non-cancer cells.

Individuals undergoing radiation therapy or treatment with one or more of chemotherapeutic drugs such as alkylating agents, antimetabolites, anthracyclines, plant alkaloids, topoisomerase inhibitors, and other antitumour agents which affect cell division or DNA synthesis and function in some way will typically benefit from protection of normally dividing non-cancer cells because the radiation and chemotherapy is not selective and will effect normally dividing non-cancer cells and well as cancer cells.

The patients in the present method have cancer. In some embodiments, the individual is identified as having cancer that lack functional guanylyl cyclase C. In some embodiments, the cancer which lacks functional guanylyl cyclase C is selected from the group consisting of: colorectal cancer which lacks functional guanylyl cyclase C, esophageal cancer which lacks functional guanylyl cyclase C, pancreatic cancer which lacks functional guanylyl cyclase C, liver cancer which lacks functional guanylyl cyclase C, stomach cancer which lacks functional guanylyl cyclase C, biliary system cancer which lacks functional guanylyl cyclase C, cancer of the peritoneum which lacks functional guanylyl cyclase C, bladder cancer which lacks functional guanylyl cyclase C, kidney cancer which lacks functional guanylyl cyclase C, cancer of the ureter which lacks functional guanylyl cyclase C, prostate cancer which lacks functional guanylyl cyclase C, ovarian cancer which lacks functional guanylyl cyclase C, uterus cancer which lacks functional guanylyl cyclase C and soft tissues of the abdomen and pelvis such as sarcomas which lack functional guanylyl cyclase C. In some embodiments, the individual is identified as having cancer that lack functional p53. In some embodiments, the cancer which lacks functional guanylyl cyclase C and functional p53. In some embodiments, the cancer is primary colorectal cancer which lacks functional p53.

### Toxic Chemotherapy

Alkylating agents are classified under L01A in the Anatomical Therapeutic Chemical Classification System. These agents function as anticancer agents by damaging DNA through their attachment to the alkyl group attached to the guanine base of DNA, at the number 7 nitrogen atom of the imidazole ring. Alkylating agents are toxic to normal cells and can cause severe side effects when used as anticancer agents. Classical alkylating agents include true alkyl groups, include the Nitrogen mustards such as Cyclophosphamide, Mechlorethamine or mustine (HN2), Uramustine or uracil mustard, Melphalan, Chlorambucil, Ifosfamidel the Nitrosoureas such as Carmustine, Lomustine, Streptozocin; and the Alkyl sulfonates such as Busulfan. Thiotepa and its analogues are often but not always considered classical. Alkylating-like Platinum-based chemotherapeutic drugs, sometimes referred to as platinum analogs, do not have an alkyl group, but nevertheless damage DNA. These compounds are sometimes described as “alkylating-like” because they coordinate to DNA to interfere with DNA repair. These agents also bind at N7 of guanine. Examples of Alkylating-like Platinum-based chemotherapeutic drugs include Cisplatin, Carboplatin, Nedaplatin, Oxaliplatin, Satraplatin, Triplatin, and tetranitrate. While the platinum agents are sometimes described as nonclassical, more typically, the nonclassical alkylating agents include procarbazine and altretamine. Tetrazines (dacarbazine, mitozolomide, temozolomide) are sometimes also listed in this category.

Antimetabolite agents are classified under L01B in the ATC system. They are toxic chemicals that inhibit the use of a metabolite that is part of normal metabolism, thus halting cell growth and cell division by interfering with DNA production and therefore cell division and the growth of tumors. Antimetabolite agents are toxic to normal dividing cells as well as cancer cells and can cause severe side effects when used as anticancer agents. Anti-metabolites include purine analogs such as azathioprine, mercaptopurine, thioguanine, fludarabine, pentostatin and cladribine; pyrimidine analogs such as 5-fluorouracil (5FU) a thymidylate synthase inhibitor, floxuridine, cytosine arabinoside (Cytarabine), and antifolates such as methotrexate, trimethoprim, pyrimethamine, pemetrexed, raltitrexed and pralatrexate.

Anthracyclines are a class of anti-cancer drugs derived from Streptomyces bacteria. Anthracycline mechanisms of action include inhibition of DNA and RNA synthesis by intercalating between base pairs of the DNA/RNA strand, and thus preventing the replication of rapidly-growing cancer cells; inhibition of topoiosomerase II enzyme, preventing the relaxing of supercoiled DNA and thus blocking DNA transcription and replication, and creation of iron-mediated free oxygen radicals that damage the DNA and cell membranes. Examples of anthracyclines include daunorubicin (Daunomycin), liposomal daunorubicin, doxorubicin (Adriamycin), liposomal doxorubicin, epirubicin, idarubicin, valrubicin, and the anthracycline analog mitoxantrone.

Alkaloids which block cell division by preventing microtubule function are useful as anticancer agents. Since microtubules are necessary for cell division, preventing their formation prevents cell division from occurring. Vinca alkaloids, which are classified under L01CA in the ATC system, bind to tubulin, and inhibit assembly of microtubules during the M phase of the cell cycle. The vinca alkaloids include vincristine, vinblastine, vinorelbine and vindesine. Colcemid and nocodazole, which are similar to vinca alkaloids, are anti-mitotic and anti-microtubule agents. drugs. Podophyllotoxin, which is classified under L01CB in the ATC system, is a plant-derived compound which is used to produce two other cytostatic drugs, etoposide and teniposide that prevent the cell from entering the GI phase (the start of DNA replication) and the S phase (the replication of DNA). Taxanes which is classified under L01CD in the ATC system, include taxane or paclitaxel (Taxol). Docetaxel is a semi-synthetic analogue of paclitaxel. Taxanes enhance stability of microtubules, preventing the separation of chromosomes during anaphase.

Some topoisomerase inhibitors are classified under L01CB in the ATC system which inhibit the topoisomerase enzymes that play essential rolls in maintaining DNA supercoiling. By upsetting proper DNA supercoiling, inhibition of either or the type I or type II topoisomerases interferes with both transcription and replication of DNA. Examples of type I topoisomerase inhibitors include camptothecins: irinotecan and topotecan. Examples of type II inhibitors include amsacrine, etoposide, etoposide phosphate, and teniposide which are semisynthetic derivatives of naturally occurring alkaloids, epipodophyllotoxins.

Other antineoplastic compounds function by generating free radicals. Examples include cytotoxic antibiotics such as bleomycin (L01DC01), plicamycin (L01DC02) and mitomycin (L01DC03).

### Toxic Radiation

Radiation therapy uses photons or charged particle to damage the DNA of cancerous cells. The damage may be direct or indirect ionizing the atoms which make up the DNA chain. Indirect ionization happens as a result of the ionization of water, forming free radicals, notably hydroxyl radicals, which then damage the DNA. Direct damage to DNA occurs through high-LET (linear energy transfer) charged particles such as proton, boron, carbon or neon ions which have an antitumor effect which is independent of tumor oxygen supply because these particles act mostly via direct energy transfer usually causing double-stranded DNA breaks. Conventional external beam radiotherapy is delivered via two-dimensional beams using linear accelerator machines. Stereotactic Radiation is a specialized type of external beam radiation therapy that uses focused radiation beams targeting a well-defined tumor using extremely detailed imaging scans.

In addition to radiation used in radiotherapy, GI syndrome and radiation sickness can occur when an individual is unintentionally exposed to large amounts of radiation such as the result of an accident or deliberate release of radioactive material. In such events, GI syndrome and radiation sickness can be prevented by administering compounds that elevate cGMP levels in gastrointestinal cells sufficient to elevate intracellular cGMP levels in gastrointestinal cells sufficient to arrest cell proliferation of gastrointestinal cells and/or maintain genomic integrity by enhanced DNA damage sensing and repair for a period sufficient to reduce damage to gastrointestinal cells and prevent GUI syndrome and/or radiation sickness. In some embodiments, the compounds that elevate cGMP levels may be administered starting immediately following exposure to radiation or, if in the case of emergency workers, prior to entering an area of high levels of radiation. In some embodiments, the compounds that elevate cGMP levels may be administered to individuals who are experiencing symptoms of radiation sickness.

### Protection of Normal-Dividing Non-Cancer Intestinal Cells

Protection of normally dividing non-cancer intestinal cells can be achieved by elevation of cGMP levels. The elevation of cGMP levels in normally dividing non-cancer intestinal cells may be achieved by administration of one or more compounds in amounts sufficient to achieve elevated cGMP levels. The one or more compounds are delivered to intestinal cells in amounts and frequency sufficient to sustain the cGMP at elevated levels prior to and during exposure to toxic chemotherapy and/or radiation.

In some embodiments, compounds which elevate cGMP do so through interaction with a cellular receptor present on the cells. GCC agonists may be delivered by routes that provide the agonist to contact the GCC expressed by intestinal cells in order to activate the receptors. In some embodiments, the compounds which elevate cGMP levels may be taken up by cell by other means. For example, cells which contain specific PDE or MRP isoforms would indicate the inhibitory compounds used. For example, cells expressing PDE5 would be protected by use of PDE5 inhibitors while cells expressing MRP5 would be protected by use of MRP5 inhibitors. In such embodiments, the compounds may be administered by any route such that they can be taken up by cells.

Regardless of the mechanism for delivery to the cell, the dose and route of delivery preferably minimizes uptake by cancer cells if the cancer cells are the type which are protected by elevated cGMP levels and if the compound used can affect such cells. In embodiments in which cGMP levels are to be increased in normal intestinal cells using GCC agonists, oral delivery to the gut is preferred. Compounds must be protected from degradation or uptake prior to reaching the gut. Many known peptide agonists of GCC are stable in the acidic environment of the stomach and will survive in active form when passing through the stomach to the gut. Some compounds may require enteric coating. In the case of GCC expression in cell lining the gut, the delivery of GCC agonist through local delivery directly to the interior of the intestinal, by oral or rectal administration for example, is particularly useful in that cells outside the gut will not be exposed to the GCC agonist since the tight junctions of intestinal tissue prevent direct passage of most GCC agonists.

The amount and duration of delivery of compounds which elevate cGMP levels in dividing, non-cancer intestinal cells are sufficient to maintain levels elevated to protective levels prior to and during exposure to toxic chemotherapy and radiation. The result will be the protection of a sufficient number of such cells through p53 mediated cell survival to effectively reduce the severity of side effects and/or allow for higher levels of chemotherapy and radiation to be used without being lethal or causing undesirable or intolerable levels of side effects.

In some embodiments the one or more compounds which increase cGMP levels is formulated as an injectable pharmaceutical composition suitable for parenteral administration such as by intravenous, intraarterial, intramuscular, intradermal or subcutaneous injection. Accordingly, the composition is a sterile, pyrogen-free preparation that has the structural/physical characteristics required for injectable products; i.e. it meets well known standards recognized by those skilled in the art for purity, pH, isotonicity, sterility, and particulate matter.

In some preferred embodiments, the one or more compounds which increase cGMP levels is administered orally or rectally and the compositions is formulated as pharmaceutical composition suitable for oral or rectal administration. Some embodiments providing the one or more compounds which increase cGMP levels are provided as suitable for oral administration and formulated for sustained release. Some embodiments providing the one or more compounds which increase cGMP levels are provided as suitable for oral administration and formulated by enteric coating to release the active agent in the intestine. Enteric formulations are described in U.S. Pat. Nos. 4,601,896, 4,729,893, 4,849,227, 5,271,961, 5,350,741, and 5,399,347. Oral and rectal formulation are taught in Remington's Pharmaceutical Sciences, 18th Edition, 1990, Mack Publishing Co., Easton Pa. which is incorporated herein by reference.

Alternative embodiments include sustained release formulations and implant devices which provide continuous delivery of. the one or more compounds which increase cGMP levels. In some embodiments, the one or more compounds which increase cGMP levels is administered topically, intrathecally, intraventricularly, intrapleurally, intrabronchially, or intracranially.

Generally, the one or more compounds which increase cGMP levels must be present at a sufficient level for a sustained amount of time to increase cGMP levels during the period the cells are potentially exposed to toxic chemotherapy or radiation. Generally, enough of the one or more compounds which increase cGMP levels must be administered initially and/or by continuous administration to maintain the concentration of sufficient to maintain elevated cGMP levels for most if not the entire period of time the patient is exposed to toxic chemotherapy or radiation. It is preferred that elevated cGMP levels sufficient to enhance p53 mediated cell survival be maintained for at least about 6 hours, preferably about for at least about 8 hours, more preferably about for at least about 12 hours, in some embodiments at least 16 hours, in some embodiments at least 20 hours, in some embodiments at least 24 hours, in some embodiments at least 36 hours, in some embodiments at least 48 hours, in some embodiments at least 72 hours, in some embodiments at least 96 hours, in some embodiments at least one week, in some embodiments at least two weeks, in some embodiments at least three weeks and up to about 4 weeks or more. It is important that the dosage and administration be sufficient for the cGMP level to be elevated in an amount sufficient for sufficient time to enhance p53 mediated cell survival such that the severity of side effects is reduced and/or the tolerable dose of chemotherapeutic or radiation can be increased. Dosage varies depending upon known factors such as the pharmacodynamic characteristics of the particular agent, and its mode and route of administration; age, health, and weight of the recipient; nature and extent of symptoms, kind of concurrent treatment, frequency of treatment, and the effect desired.

In some embodiments, a GCC agonist such as a peptide having SEQ ID NO:2, 3 or 5-60 is administered to the individual. In practicing the method, the compounds may be administered singly or in combination with other compounds. In the method, the compounds are preferably administered with a pharmaceutically acceptable carrier selected on the basis of the selected route of administration and standard pharmaceutical practice. It is contemplated that the daily dosage of a compound used in the method will be in the range of from about 1 microgram to about 10 grams per day. In some preferred embodiments, the daily dosage compound will be in the range of from about 10 mg to about 1 gram per day. In some preferred embodiments, the daily dosage compound will be in the range of from about 100 mg to about 500 mg per day. It is contemplated that the daily dosage of a compound used in the method that is the invention will be in the range of from about 1 μg to about 100 mg per kg of body weight, in some embodiments, from about 1 μg to about 40 mg per kg body weight; in some embodiments from about 10 μg to about 20 mg per kg per day, and in some embodiments 10 μg to about 1 mg per kg per day. Pharmaceutical compositions may be administered in a single dosage, divided dosages or in sustained release. In some preferred embodiments, the compound will be administered in multiple doses per day. In some preferred embodiments, the compound will be administered in 3-4 doses per day. The method of administering compounds include administration as a pharmaceutical composition orally in solid dosage forms, such as capsules, tablets, and powders, or in liquid dosage forms, such as elixirs, syrups, and suspensions. Compounds may be mixed with powdered carriers, such as lactose, sucrose, mannitol, starch, cellulose derivatives, magnesium stearate, and stearic acid for insertion into gelatin capsules, or for forming into tablets. Both tablets and capsules may be manufactured as sustained release products for continuous release of medication over a period of hours. Compressed tablets can be sugar or film coated to mask any unpleasant taste and protect the tablet from the atmosphere or enteric coated for selective disintegration in the gastrointestinal tract. In some preferred embodiments, compounds are delivered orally and are coated with an enteric coating which makes the compounds available upon passing through the stomach and entering the intestinal tract, preferably upon entering the large intestine. U.S. Pat. No. 4,079,125, which is incorporated herein by reference, teaches enteric coating which may be used to prepare enteric coated compound of the inventions useful in the methods of the invention. Liquid dosage forms for oral administration may contain coloring and flavoring to increase patient acceptance, in addition to a pharmaceutically acceptable diluent such as water, buffer or saline solution. For parenteral administration, a compound may be mixed with a suitable carrier or diluent such as water, an oil, saline solution, aqueous dextrose (glucose), and related sugar solutions, and glycols such as propylene glycol or polyethylene glycols. Solutions for parenteral administration contain preferably a water-soluble salt of the compound. Stabilizing agents, antioxidizing agents and preservatives may also be added. Suitable antioxidizing agents include sodium bisulfite, sodium sulfite, and ascorbic acid, citric acid and its salts, and sodium EDTA. Suitable preservatives include benzalkonium chloride, methyl- or propyl-paraben, and chlorbutanol

### Sensitizing Activity in Some Cancers

As noted above, cGMP promotes cell death in response to DNA damage by chemotherapy or radiation therapy in a variety of cancer cells including lung, breast, prostate, colorectal, and liver cancer cells. In view of the tissue specific effect of cGMP on cell death in the intestine, increase in cGMP in intestinal cells in conjunction with chemotherapy or radiation therapy to reduce GI side effects and in some cases may potentiate the therapeutic efficacy for lung, breast, prostate, colorectal, and liver cancers.

In the treatment of cancer of a type which is rendered more susceptible to chemotherapy- or radiotherapy-induced cell death when cGMP levels are elevated, compounds which elevate cGMP may be administered in doses and by routes of administration a manner which delivered sufficient compound to cancer cells to increase the effectiveness of chemotherapy and radiotherapy to kill the cancer cells. In some embodiments, the compounds may potentiate chemotherapy- or radiotherapy-induced cell death in cancer cells while protecting non-cancer cells from chemotherapy or radiation therapy through p53 mediated cell survival.

### Other Cell Types

In some embodiments, the normal non-dividing cells may be other types of cells for which elevated cGMP can enhance p53 mediated cell survival. In some embodiments, the normal non-dividing cells may be hair follicles, skin, lungs, nasal passages, other mucosae or tissue in the oral cavity. Compounds may be delivered topically to the scalp or to tissue of the oral cavity including mouth, tongue, gums, and buccal tissue, preferably formulated for local uptake with minimal system uptake. Compounds may be delivered using an inhalation device and/or nasal spray, preferably formulated for local uptake with minimal system uptake. Similarly, compounds which elevate cGMP levels in normal dividing non-cancer cells such as other cells of the mucosae or such as skin cells may be formulated for preferential uptake and delivered directly to such cells. Such delivery may include intraocularly, intravaginally, intraurethraly, rectal/anal or topically.

The amount and duration of delivery of compounds which elevate cGMP levels in dividing, non-cancer cells which can be protected by p53 mediated cell survival by elevated cGMP is sufficient to maintain levels elevated to protective levels prior to and during exposure to toxic chemotherapy and radiation. The result will be the protection of a sufficient number of such cells by p53 mediated cell survival to effectively reduce the severity of side effects and/or allow for higher levels of chemotherapy and radiation to be used without being lethal or causing undesirable or intolerable levels of side effects.

### EXAMPLES

### Example 1

Therapeutic radiation and genotoxic chemotherapeutics are part of the armamentarium of cancer treatment. These genotoxic agents are generally limited in their dose by damage to normal tissues. We have discovered that the cell signaling molecule cyclic GMP can prevent genotoxic damage to cells through a p53-dependent mechanism. Here, we describe a method to improve colorectal tumor treatment with radiation or chemotherapy by identifying tumors that are either GUCY2C-negative or carry mutant p53. For these tumors, GUCY2C activating agents (e.g., ST, linaclotide (SEQ ID NO:59, plecanatide SEQ ID NO:60) can be used to spare normal intestinal epithelium without impacting the therapeutic efficacy of genotoxic agents (e.g., radiation, chemotherapy). In this way, higher doses of genotoxic therapy can be applied to kill tumor s without causing normal tissue damage. Additionally, provided herein are methods to improve extra-intestinal tumor therapy, for tumors with mutant p53, using genotoxic agents in combination with agents that elevate cyclic GMP in tissues (e.g., nitric oxide, natriuretic peptides, phosphodiesterase inhibitors) to increase the therapeutic dose of these agents while sparing normal tissues with wild type p53.

Currently, there are no cytoprotective agents that permit selective killing of tumors but selective sparing of normal tissues. This discovery leverages unique insights into the cytoprotective effects of cyclic GMP, and its dependence on wild type p53, to achieve this unique selectivity. Currently, one of the greatest limitations to anti-tumor therapy is the therapeutic window—the difference between doses that kill tumor and those that kill normal tissues. This invention provides an opportunity to improve that therapeutic window.

In some embodiments, for tumors arising in the intestine-if they are negative for GCC or mutant for p53, GCC (also referred to as GUCY2C) ligands are used to create resistance in the normal intestinal epithelium but maintain the genotoxic effects in the tumor. This improves the therapeutic window of the genotoxic therapy.

In some embodiments, for tumors arising outside the intestine, and which carry a mutation in p53, agents that elevate cyclic GMP in tissues (e.g., nitric oxide-generating agents, natriuretic peptides and analogs, phosphodiesterase inhibitors) are used to improve the therapeutic window for genotoxic therapies that would permit tumor cell killing but spare normal tissues.

The therapeutic window is the rate limiting factor in nearly all tumor cell therapeutic paradigms.

High doses of ionizing radiation induce acute damage to epithelial cells of the gastrointestinal (GI) tract, mediating toxicities restricting the therapeutic efficacy of radiation in cancer and morbidity and mortality in nuclear disasters. There is no approved prophylaxis or therapy, in part reflecting an incomplete understanding of mechanisms contributing to the acute radiation induced GI syndrome (RIGS). Guanylate cyclase C (GUCY2C) and its hormones guanylin and uroguanylin have recently emerged as one paracrine axis defending intestinal mucosal integrity against mutational, chemical, and inflammatory injury. Here, we reveal a role for the GUCY2C paracrine axis in compensatory mechanisms opposing RIGS. Eliminating GUCY2C signaling exacerbates RIGS, amplifying radiation-induced mortality, weight loss, mucosal bleeding, debilitation and intestinal dysfunction. In that context, durable expression of GUCY2C, guanylin and uroguanyin mRNA and protein by intestinal epithelial cells was preserved following lethal irradiation inducing RIGS. Moreover, oral delivery of the heat-stable enterotoxin (ST), an exogenous GUCY2C ligand, opposed RIGS, a process requiring p53 activation mediated by dissociation from MDM2. In turn, p53 activation prevented cell death by selectively limiting mitotic catastrophe, but not apoptosis. These studies reveal a role for the GUCY2C paracrine hormone axis as a novel compensatory mechanism opposing RIGS. They highlight the potential of oral GUCY2C agonists (Linzess™; Trulance™) to prevent and treat RIGS in cancer therapy and nuclear disasters.

Introduction

Exposure to radiation in the context of terrorist attacks or natural disasters produces death within about 10 days reflecting toxicity to the gastrointestinal (GI) tract, constituting the acute radiation-induced GI syndrome (RIGS)(1-3). In contrast to radiation-induced bone marrow toxicity, in which death can be prevented by bone marrow transplantation, there are no approved management paradigms to prevent or treat RIGS (4). Importantly, radiation therapy remains a mainstay in the management of cancer, a leading cause of death worldwide.

Radiation therapy destroys rapidly proliferating cancer cells and, inevitably, normal tissues characterized by continuous regeneration programs, including hair follicles, bone marrow, the GI tract as well as other glandular epithelia (5). In that context, dose-limiting toxicities of radiation discourage patients from completing therapy; restrict maximum doses of radiation which limits the efficacy of treatment; and can lead to chronic morbidity and mortality (5). Inadequate management in part reflects an incomplete understanding of mechanisms underlying RIGS. Indeed, critical molecular mechanisms and cellular targets mediating epithelial toxicity underlying RIGS remain controversial (6-14). Recent studies suggest that p53 in intestinal epithelial cells principally controls radiation-induced GI toxicity in mice, independently of apoptosis (7). In that context, deletion of the intrinsic apoptotic pathway from intestinal endothelial or epithelial cells failed to protect mice from GI toxicity-related death (7).

In contrast, tissue-specific targeted deletion of intestinal epithelial cell p53 exacerbates, while its over-expression rescues, RIGS in mice (7,14). However, mechanisms underlying radiation induced intestinal epithelial cell death and intestinal mucosa damage remain undefined (7). GUCY2C is the intestinal receptor for the endogenous paracrine hormones guanylin (GUCA2A) and uroguanylin (GUCA2B) and the heat-stable enterotoxins (STs) produced by diarrheagenic bacteria (5-17). This signaling axis plays a central role in mucosal physiology, regulating fluid and electrolyte secretion (15,16), and in coordinating crypt-surface homeostasis, regulating enterocyte proliferation, differentiation, metabolism, apoptosis, DNA repair, and epithelial mesenchymal cross-talk (18-20). Further, this axis maintains the intestinal barrier, opposing epithelial injury induced by carcinogens, inflammation, and radiation, and its dysfunction contributes to the pathophysiology of inflammatory bowel disease and tumorigenesis (19-30). While the GUCY2C signaling axis has emerged as one guardian of intestinal epithelia integrity, the role of this axis in responses to lethal radiation, and its utility as a therapeutic target to prevent and treat RIGS remains undefined (22).

Here, we define a novel role for the GUCY2C paracrine hormone axis in compensatory responses opposing RIGS. Indeed, eliminating GUCY2C signaling amplifies radiation-induced GI toxicity. In that context, durable expression of GUCY2C, GUCA2A, and GUCA2B mRNA and protein is preserved following high doses of radiation that induce RIGS. Moreover, oral administration of the GUYC2C ligand ST opposed RIGS through a p53-dependent mechanism associated with the rescue of intestinal epithelial cells selectively from mitotic catastrophe, but not from apoptosis. These observations reveal a previously unrecognized compensatory mechanism to epithelial injury induced by high-dose radiation, involving signaling by the GUCY2C paracrine axis that opposes RIGS. They highlight the potential for oral GUCY2C targeted agents to prevent or treat RIGS in the setting of cancer radiotherapy or environmental exposure through nuclear accident or terrorism. The opportunity to immediately translate these approaches is underscored by the recent regulatory approval of linactotide (Linzess™) and plecanatide (Trulance™), oral GUCY2C ligands that treat chronic constipation (31).

### Materials and Methods

Animal Models

Mice with a targeted germline deletion of GUCY2C (Gucy2c−/−) are well-characterized, and were used after >14 generations of backcrossing onto the C57BL/6 background (15,16,18-20,26,32). p53FL-vil-Cre-ERT2 mice were generated by crossbreeding vil-Cre-ERT2 (provided by S. Robine, Institut Curie-CNRS, France) with p53FL transgenic mice (mixed FVB.129 and C57BL/6 backgrounds, kindly provided by Dr. Karen Knudsen, Thomas Jefferson University, Philadelphia, Pa.). Biallelic loss of p53 in intestinal epithelial cells (p53int−/−) was induced by IP administration of tamoxifen (75 mg tamoxifen/kg/d×5 d) to F2 p53FL-vil-Cre-ERT2 and control littermate p53+-vil-Cre-ERT2, and deletion confirmed structurally by immunoblot analysis of phosphorylated p53 and functionally by radiation-induced mortality. All experiments were carried out with mice that were between 2 to 3.5 months old (mixed males and females) and all mice were on mixed genetic backgrounds as described above. Where appropriate, age-matched and litternate controls were utilized to minimize the effect of genetic backgrounds. C57BL/6 mice used in oral ST or control peptide supplementation studies were obtained from NIH (NCI-Frederick) while those used for GUCY2C and ligand expression analysis were obtained from the Jackson Laboratory (Bar Harbor, Me.). This study was approved by the Institutional Animal Care and Use Committee of Thomas Jefferson University (protocol 01518).

Gamma Irradiation-Induced GI Toxicity

Anesthetized mice were irradiated with total-body gamma irradiation (TBI) or with back limbs to tail and front limbs to head shielded with lead covers for subtotal-body irradiation (STBI) with exposure of abdominal area (approximately 1 inch2 from xiphoid to pubic symphysis). Mice were irradiated with a 137Cs irradiator (Gammacell 40) at a dose rate of approximately 70 cGy/min for different doses from 8 to 25 Gy/mice. Mice had free access to regular food and water before and after irradiation. The severity of GI toxicity was evaluated by mortality, debilitation (untidy fur coats), body weight, visible diarrhea, fecal occult blood, stool formation, stool water accumulation, and histopathology.

ST and Control Peptides

ST1-18 and control peptide (CP; inactive ST analog contains the same primary amino acid sequence, but with cysteines at positions 5, 6, 9, 10, 14, 17 replaced by alanine) were purchased from Bachem Co. (customer order; purity >99.0%). ST and control peptides were resuspended in 1× phosphate-buffered saline (PBS) at a concentration of 50 ng/μL. Mice were orally gavaged with 10 μg of CP or ST (in 200 μL solution) using a feeding needle (cat. #01-208-88, Fisher Scientific) (26) daily for 14 d before and 14 d after irradiation. ST and CP were prepared by solid phase synthesis and purified by reverse phase HPLC, their structure confirned by mass spectrometry by Bachem Co. (customer order; purity >99.0%), and their activities confirmed by quantifying competitive ligand binding, guanylate cyclase activation and secretion in the suckling mouse assay (16,33).

Reagents

McCoy's 5A and Dulbecco's Modified Eagle Medium (DMEM) containing 10% fetal bovine serum and other reagents for cell culture were obtained from Life Technologies (Rockville, Md.). 8-Bromoguanosine 3′, 5′-cyclic monophosphate (8-Br-cGMP), a cell-permeant analog of cGMP, was obtained from Sigma (St. Louis, Mo.) and 500 μM was used in all experiments (18,20,25,26,34).

Cell Lines

C57BL/6-derived EL4 lymphoma cells (lymphoblasts in mouse thymus; thymoma) and C57BL/6-derived B16 melanoma cells were obtained from ATCC. HCT116 (wild-type p53) human colon cancer cells, which lack GUCY2C ((19,34,35), were purchased from ATCC. Isogenic HCT116-p53-null cells were a gift from Dr. Bert Vogelstein (Johns Hopkins University, MD) (36).

Ectopic Tumor Seeding and Growth Measurement

EL4 and B16 cells (104 cells per injection) were injected subcutaneously in mouse flanks (EL4, left and B16, right). Tumor growth was measured once every 3 d and tumor volume was calculated by multiplying 3 tumor dimensions. No significant differences in tumor growth before and after subtotal body irradiation was observed in mice treated with ST compared to CP.

Immunoblot Analyses

Protein was extracted from mouse small intestine and colon mucosa in T-Per reagent (Pierce, Dallas, Tex.), or from in vitro cell lysates in Laemmli buffer, and supplemented with protease and phosphatase inhibitors (Roche, Indianapolis, Ind.). Protein was quantified by immunoblot analysis employing antibodies to: phosphorylated histone H2AX (cat. #2577, 1:200 dilution), phosphorylated p53 (cat. #9284, 1:200 dilution), cleaved caspase 3 (cat. #9579, 1:200 dilution), Mdm2 (cat. #3521, 1:200 dilution), and GAPDH (cat. #2118, 1:200 dilution) from Cell Signaling Technology (Danvers, Mass.), phosphorylated histone H2AX (cat. #05-636, 1:1000 dilution) from Millipore (Billerica, Mass.), and p53 (cat. #sc-126, 1:1000 dilution) from Santa Cruz (Santa Cruz, Calif.). The antibody to GUCY2C was validated previously (25,26). Antisera to GUCA2A and GUCA2B were generously provided by Dr. Michael Goy (University of North Carolina, Chapel Hill, N.C.) (37,38). Secondary antibodies conjugated to horseradish peroxidase were from Jackson Immunoresearch Laboratories (West Grove, Pa.). Staining intensity of specific bands quantified by densitometry was normalized to that for GAPDH using a Kodak imaging system. Average relative intensity reflects the mean of at least three animals in each group and the mean of at least two independent experiments. Molecular weight markers (Cat. #10748010, 5 μL per run, or Cat. #LC5800, 10 μL per run) for immunoblot analyses were from Invitrogen (Grand Island. N.Y.). Secondary antibodies specific to light chains, including goat anti-mouse IgG (cat. #115-065-174) and mouse anti-rabbit IgG (cat. #211-062-171), were from Jackson Immunoresearch Laboratories (Suffolk, UK) for immunoblot analysis following immunoprecipitation.

### Immunoprecipitation

Protein from 8-10×106 HCT116 cells was extracted in 1% NP40 imnunoprecipitation (IP) lysis buffer supplemented with protease and phosphatase inhibitors and incubated with antibodies to Mdm2 (cat. #3521, 5 μg) from Cell Signaling Technology and p53 (cat. #sc-126, 1 μg) from Santa Cruz and protein A beads (Invitrogen, Grand Island, N.Y.) overnight followed by six washes. Precipitated proteins were collected in Laemmli buffer (with 5% beta mercaptoethanol) supplemented with protease and phosphatase inhibitors (Roche) and quantified by immunoblot analysis employing antibodies to Mdm2 (cat. #3521, 1:200 dilution) from Cell Signaling Technology and p53 (cat. #sc-126, 1:1000 dilution) from Santa Cruz. Mouse IgG (5 μg, cat. #10400C, Invitrogen) and rabbit IgG (5 μg, cat. #10400C, Invitrogen) were isotype controls for immunoprecipitation.

### Immunohistochemistry and Immunofluorescence

Antigens were unmasked in paraffin-embedded sections (5 μm) by heating at 100° C. for 10 min in 10 mM citric buffer, ph 6.0. In addition to those already described, antibodies to antigens probed here included: phosphorylated histone H2AX from Cell Signaling (cat. #2577, 1:200 dilution), or from Millipore (cat. #05-636, 1:1000 dilution), cleaved caspase 3 from Cell Signaling (cat. #9579, 1:200 dilution), and β-catenin from Santa Cruz (cat. #sc-7199, 1:50 dilution). The antibody to GUCY2C (25,26) and the antisera to GUCA2A and GUCA2B were described previously (37,38). Fluorescent secondary antibodies were from Invitrogen. Tyramide signal amplification was used to detect GUCY2C and GUCA2A; secondary antibodies conjugated to horseradish peroxidase were from Jackson Immunoresearch Laboratories (cat #115-035-206 and #111-036-046, 1:1000 dilution), and fluorescein-conjugated tyramine was prepared from tyramine HCl (cat #T2879, Sigma) and NHS-fluorescein (cat #46410, Thermo Scientific)(39).

Phosphorylated histone H2AX-positive cells were quantified in 200-1000 crypts per section per animal and positive cells normalized to crypt number. Results reflect the means±SEM if at least 3 animals in each group. Immunofluorescence stains were performed in HCT116 and HCT116 p53-null cells using antibodies to the following antigens included: α/β-tubulin from Cell Signaling (cat. #2148, 1:200 dilution) and γ-tubulin from Abcam (cat. #ab11317, 1:100 dilution, Cambridge, Mass.). Fluorescence images were captured with an EVOS FL auto cell imaging system from Life Technologies-Thermo Fischer Scientific (Waltham, Mass.).

### Cell Treatment, Irradiation and Colony Formation Assay

HCT116 and HCT116 p53-null cells were plated in 6-well dishes at 1×104 cells/well followed by treatment for 7 d with vehicle or cell permeable cGMP (8-Br-cGMP, 500 μM). Media containing different treatments were changed every other day. After exposure to radiation (0-4 Gy) cells were trypsinized and plated in 6-well dishes at different densities depending on the potency of the treatments (104 cells/well for HCT116 exposed at 0, 1 and 2 Gy; 4×104 cells/well for HCT116 p53-null exposed at 0, 1 and 2 Gy; 50×104 cells/well for HCT116 exposed at 3 and 4 Gy; 200×104 cells/well for HCT116 p53-null exposed at 3 and 4 Gy). Cells were treated with vehicle or 8-Br-cGMP for 7 d after irradiation, then fixed and stained with 10% methylene blue in 70% ethanol. The number of colonies, defined as >50 cells/colony were counted, and the surviving fraction was calculated as the ratio of the number of colonies in the treated sample to the number of colonies produced by cells that were not irradiated. Triplicates were used for each condition in three independent experiments.

### Anaphase Bridge Index (ABI) and Aneuploidy

Cells preconditioned with 8-Br-cGMP, or control cells, were irradiated (5 Gy), then seeded on coverslips in 24-well plates (5×104 cells per well). ABI and aneuploidy were quantified 2 d after irradiation.

ABI: Cells were fixed in 4% PFA and stained with DAPI. Anaphase cells were analyzed and abnormal anaphase cells were calculated under a fluorescence microscope. More than 200 anaphase cells were analyzed in each treatment group in each independent experiment. Any abnormal anaphase cells with anaphase bridges or anaphase lag showing extended chromosome bridging between two spindle poles were enumerated and the ABI was calculated as percentage of abnormal anaphase cells over total anaphase cells.

Aneuploidy: Cells were fixed in 4% PFA and stained with DAPI, and immunofluorescence stains were performed using α/β-tubulin-specific antibodies and centromere-specific γ-tubulin antibodies, detected with Alexa Fluor® 555 or Alexa Fluor® 488 labeled secondary antibodies from Invitrogen. Images were acquired with a laser confocal microscope (Zeiss 510M and Nikon C1 Plus, Thomas Jefferson University Bioimaging Shared Resource), and 0.5 μm optical sections in the z axis were collected with a 100×1.3 NA oil immersion objective at room temperature. Iterative restoration was performed using LSM Image Brower (Zeiss), and images represent three to four merged planes in the z axis. Abnormal anaphase chromatids were counted if cells contained more than two centrosomes or two centrosomes located in the same direction to the spindle midzone separated from kinetochores at the poles.

### Quantitative RT-PCR Analysis

Transcripts for GUCY2C, GUCA2A, and GUCA2B were quantified by RT-PCR employing primers and conditions described previously (25,26).

### 125I-Labeled ST Binding

Binding of 125I-labeled ST to GUCY2C was performed as described previously (33). Briefly, membranes were prepared from cells as described previously (33) and ST was iodinated (125ITyr4-ST) to a final specific activity of 2,000 Ci/mmol (33). Total binding was measured by counts per minute (CPM) in the absence of unlabeled ST competition, whereas nonspecific binding was measured in the presence of 1×10−5 M unlabeled ST. Specific binding was calculated by subtracting nonspecific binding from total binding (33). Assays were performed at least in triplicate.

### Statistical Analyses

Statistical significance was determined by unpaired two-tailed Student's t test unless otherwise indicated. Results represent means t SEM from at least 3 animals or 3 experiments performed in triplicate. Survival and disease-free survival were analyzed by Kaplan-Meier analysis. Body weight was analyzed using a frailty model combining a segmented linear longitudinal model of body weight, a log-normal model for survival time, and a log-normal model for random break point for body weight (inflection point). Analyses of fecal occult blood and untidy fur were performed by Cochran-Mantel-Hansel test. Colony formation was analyzed by a pairwise comparison in four treatments with isotherm slopes by linear regression.

### Results

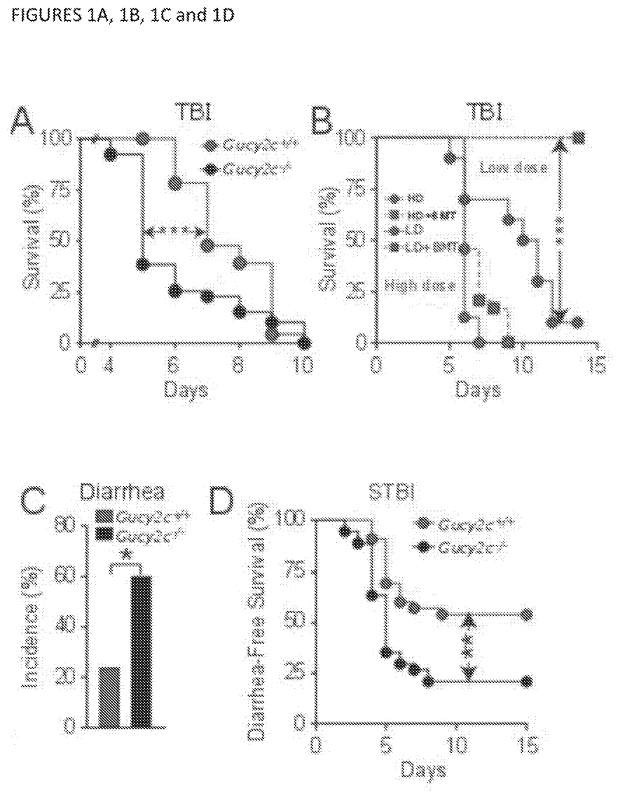

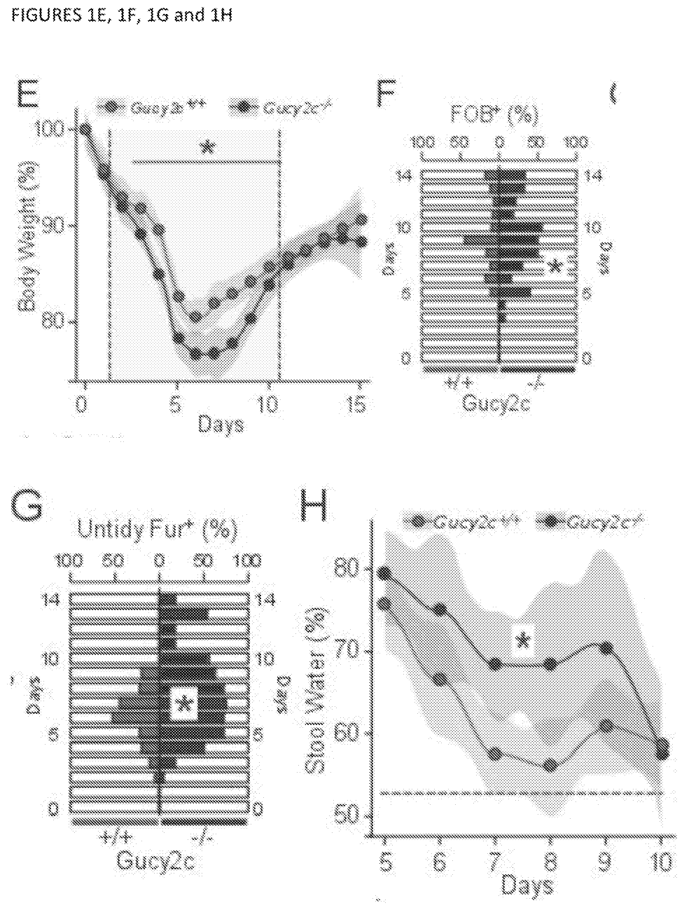

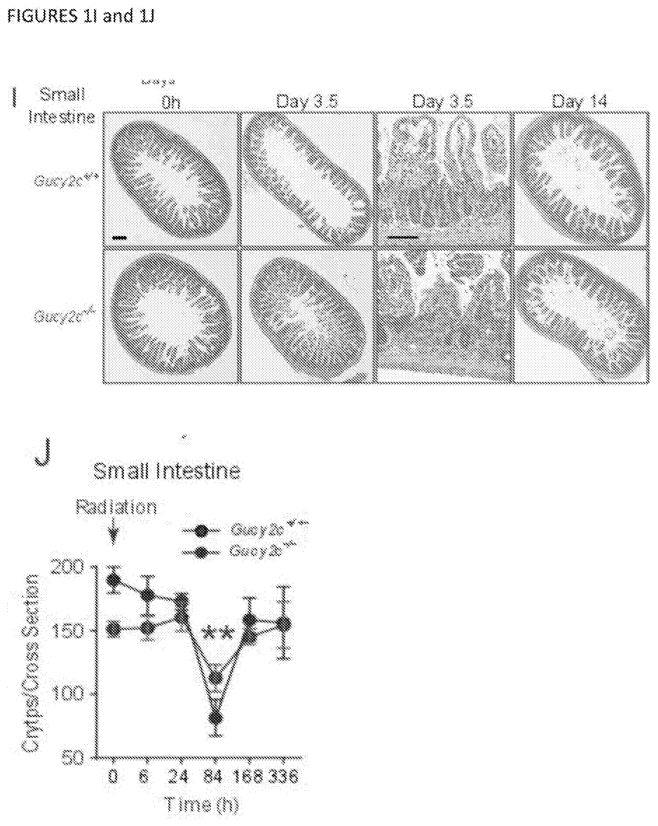

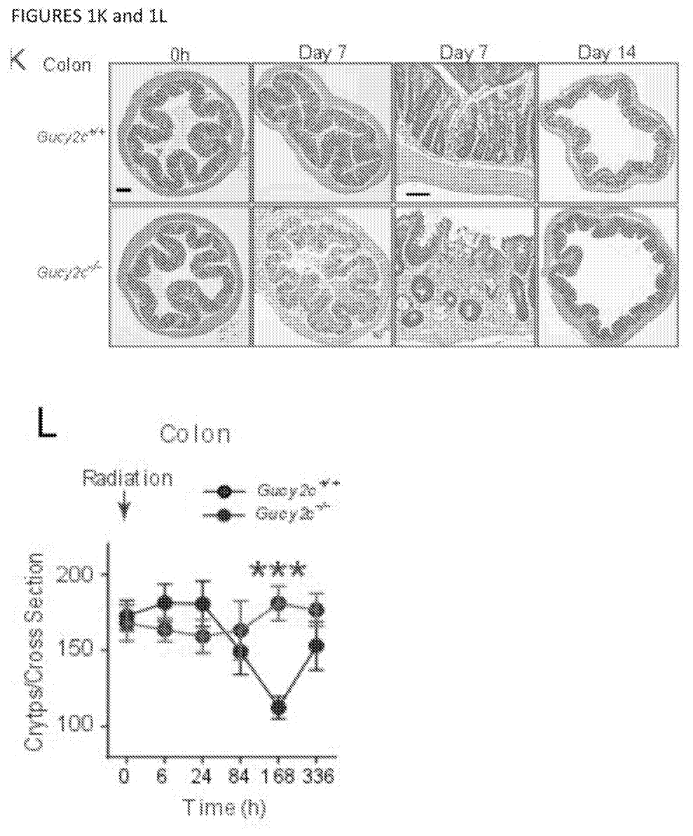

Silencing GUCY2C exacerbates RIGS. A role for GUCY2C in opposing epithelial cell apoptosis induced by low doses of ionizing radiation (22) suggested that this receptor may play a role in RIGS. Targeted germline deletion of Gucy2c (Gucy2c−/− mice) (15,16,18-20,26,32) accelerated the death of mice following exposure to a lethal dose (high dose, 15 Gy) of total body irradiation (TBI; FIG. 1A). This dose of radiation produced death by inducing RIGS which could not be rescued by bone marrow transplantation, in contrast to low dose (8 Gy) radiation which produced the hematopoietic, but not the GI, syndrome (FIG. 1B). Similarly, silencing GUCY2C signaling exacerbated acute GI toxicity quantified by diarrhea (FIG. 1C) and decreased survival (FIG. 1D) following 18 Gy sub-total abdominal irradiation (STBI), with bone marrow preservation by shielding. Exacerbation of RIGS in the absence of GUCY2C signaling was associated with increased intestinal dysfunction, including weight loss (FIG. 1E), intestinal bleeding (FIG. 1F), debilitation (untidy fur, FIG. 1G) (40), and stool water accumulation (FIG. 1H) (41). Silencing GUCY2C signaling amplified intestinal epithelial disruption, quantified as crypt loss, produced by STBI in small intestine (FIG. 1I-J). Moreover, it created novel epithelial vulnerability in the colon, which is relatively resistant to RIGS (FIG. 1K-L) (1-5). Together, these observations reveal that the GUCY2C signaling axis plays a compensatory role in modulating mechanisms contributing to RIGS.

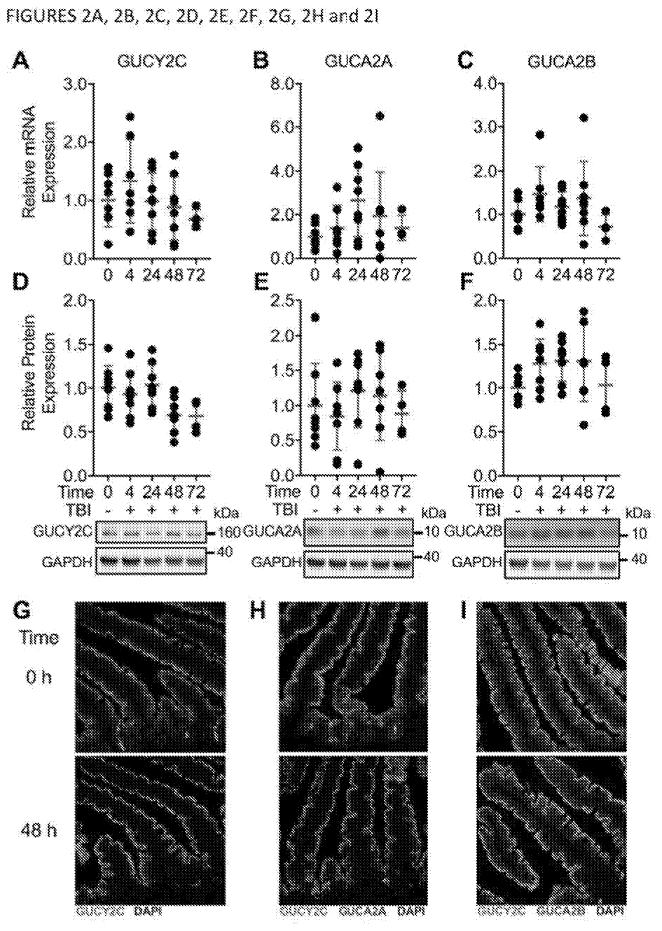

GI-toxic irradiation preserves durable expression of GUCY2C and its paracrine hormones. A role for the GUCY2C paracrine hormone axis in compensatory mechanisms opposing RIGS is predicated on the persistence of expression of the receptor and its hormones following high doses of radiation. Indeed, GUCY2C mRNA and protein, characteristically expressed along dhe entire crypt-villus axis (17), was durably preserved following lethal TBI (FIG. 2A, D, G), a result that is similar to other conditions disrupting epithelial integrity, including tumorigenesis (18-20,25). Unexpectedly, GI-toxic TBI preserved expression of GUCA2A (FIG. 2B, E, H) and GUCA2B (FIG. 2C, F, I), in contrast to other modes of disrupting epithelial integrity, including tumorigenesis, inflammatory bowel disease, and metabolic stress in which ligand expression is lost (18-21,25,26). Indeed, expression of GUCA2A, which is low in small intestine, was retained primarily in isolated epithelial cells, as described previously (42). In contrast, expression of GUCA2B, which is the predominant GUCY2C hormone in the small intestine, was retained principally by differentiated epithelial cells in distal villi (42). Preservation of receptor and hormone expression was durable, and there were no significant differences in mRNA or protein levels across the time course of injury response (FIG. 2). Moreover, preservation of hormone expression was independent of GUCY2C expression. These observations are consistent with a role for the GUCY2C paracrine hormone signaling axis in compensatory responses that oppose acute radiation-induced GI toxicity.

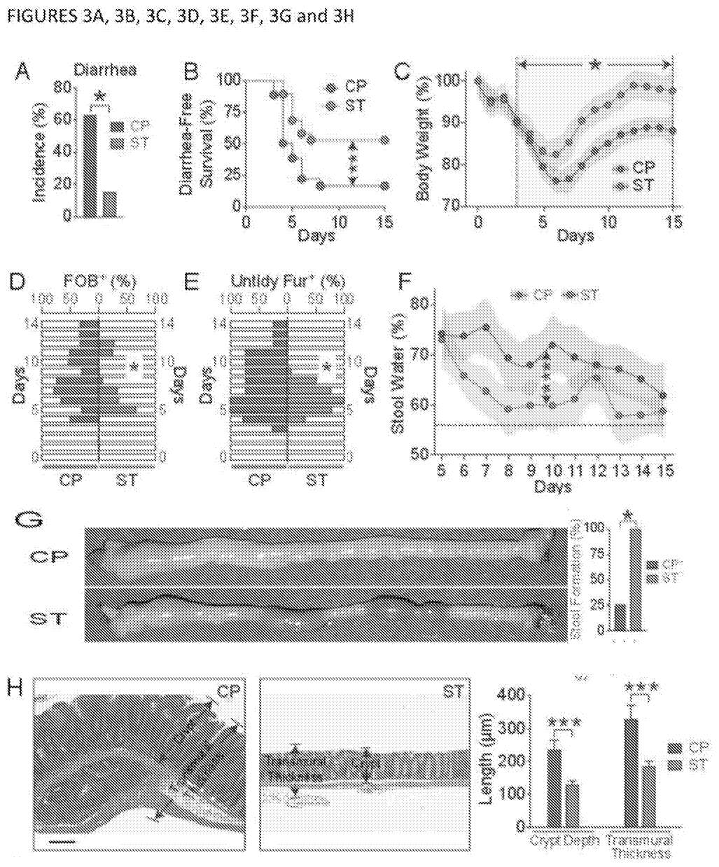

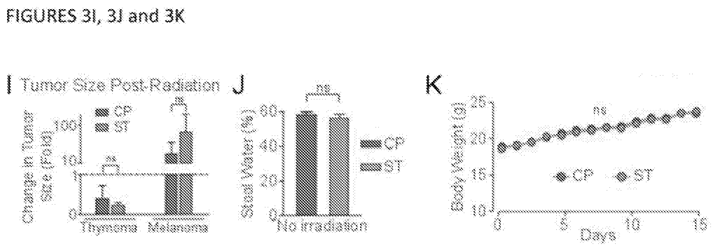

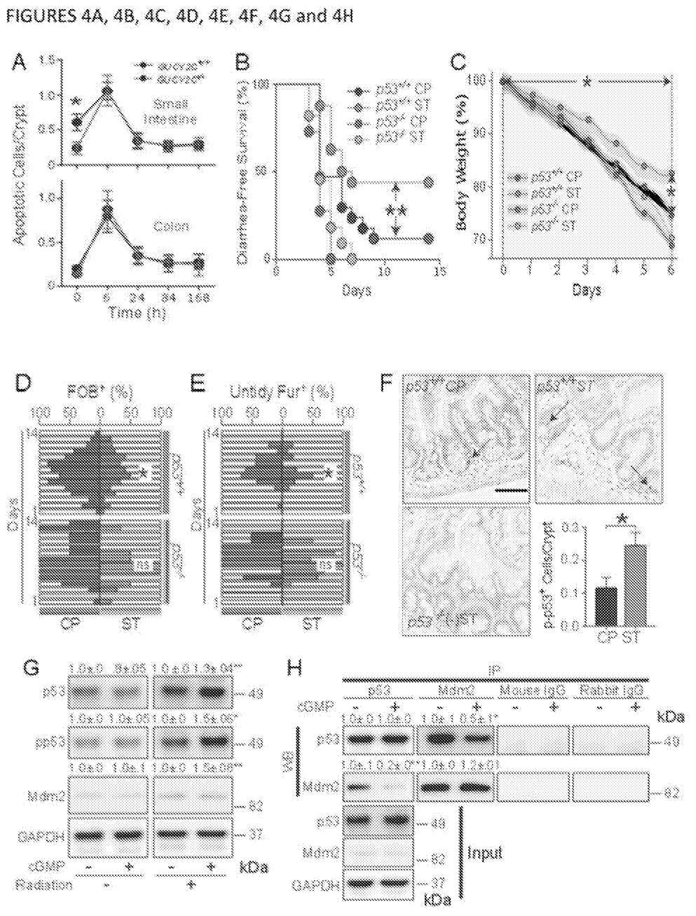

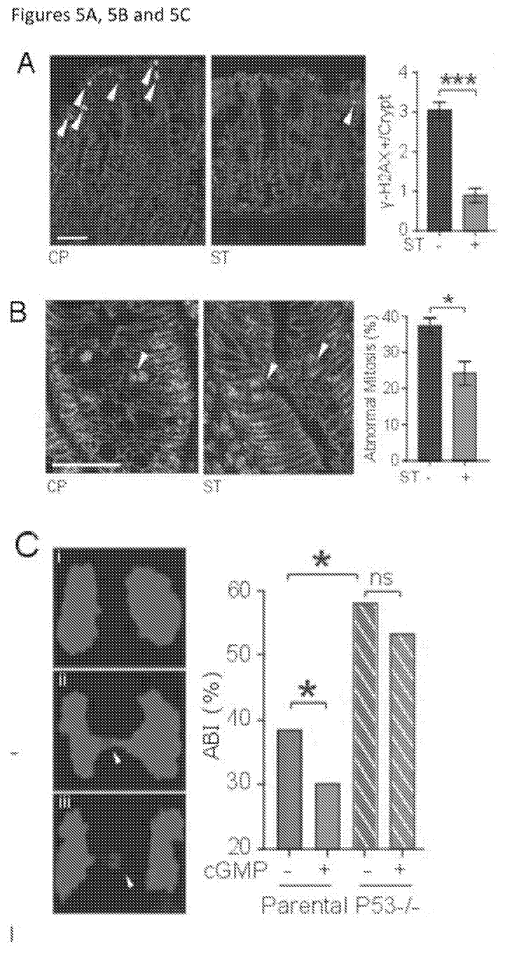

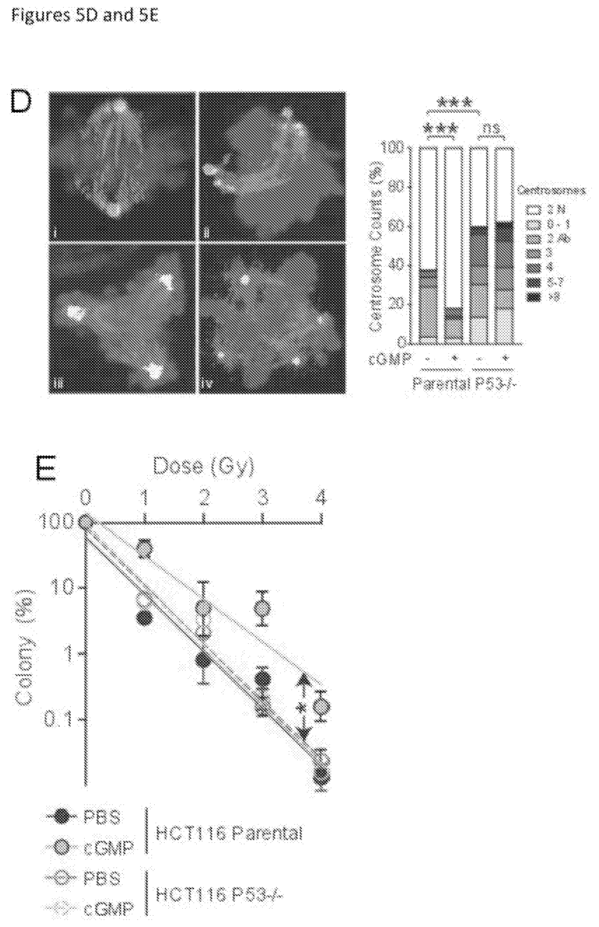

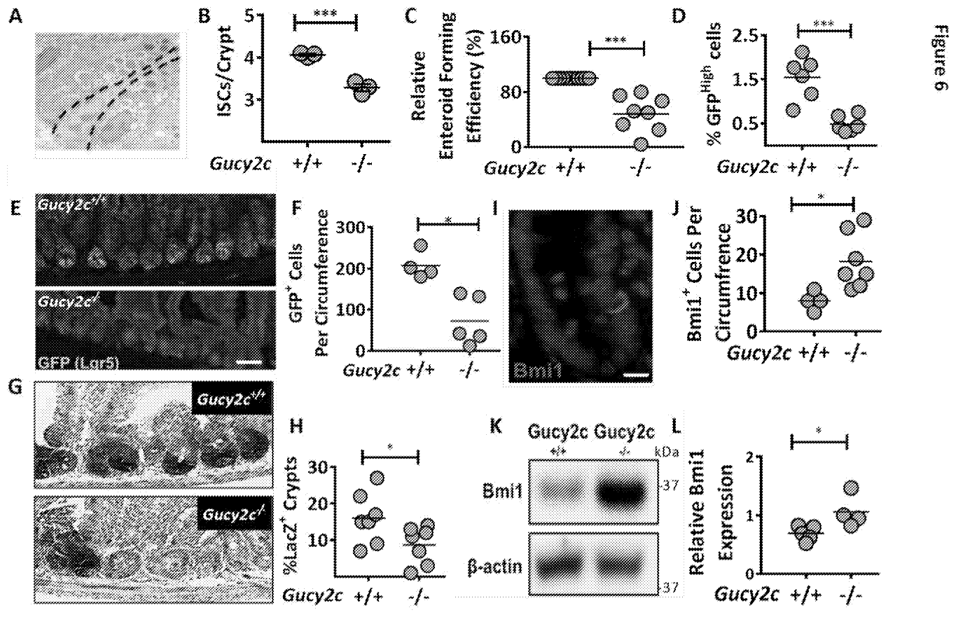

Moreover, the persistence of receptor expression across the continuum of injury response suggests the potential utility of GUCY2C as a therapeutic target to prevent RIGS. GUCY2C activation by oral ligand rescues RIGS, but not extra-GI tumor responses to radiation. In wild type mice, oral administration of ST, an exogenous GUCY2C ligand, reduced morbidity and mortality induced by STBI, quantified by the incidence of diarrhea (FIG. 3A) and survival (FIG. 3B) respectively. Similarly, oral ST opposed STBI-induced intestinal dysfunction, including weight loss (FIG. 3C), intestinal bleeding (FIG. 3D), debilitation (FIG. 3E), and stool water accumulation (FIG. 3F). Further, oral ST rescued intestinal morphology and stool formation (FIG. 3G), and water reabsorption associated with preservation of normal histology (FIG. 3H) after STBI. In contrast, oral ST did not rescue RIGS in Gucy2c−/− mice. Furthermore, oral ST did not alter therapeutic radiation responses of radiation-sensitive thymoma or radiation-resistant melanoma (FIG. 3I). Moreover, chronic oral ST was safe, without adverse pharmacological effects like diarrhea (FIG. 3J) or growth retardation (FIG. 3K). These observations support the suggestion that GUCY2C signaling comprises a compensatory mechanism opposing RIGS that can be engaged by orally administered ligands. Indeed, GUCY2C ligands safely protect intestinal epithelial cells specifically (26), without altering therapeutic responses to radiation of tumors outside the intestine. GUCY2C signaling opposing RIGS requires p53. GUCY2C signaling protects intestinal epithelial cells from apoptosis induced by low dose radiation (22). However, while silencing GUCY2C signaling increased basal levels of apoptosis in small intestine, as demonstrated previously (18), it did not alter apoptosis associated with RIGS along the rostral-caudal axis of the intestine across the continuum of injury responses (FIG. 4A). In that context, p53 also opposes RIGS through mechanisms that are independent of apoptosis (7). Indeed, eliminating p53 phenocopied GUCY2C silencing, exacerbating RIGS-related mortality (FIG. 1D). Further, activation of GUCY2C with oral ST improved survival in wild type, but not p53int−/−, mice following STBI (FIG. 4B). Moreover, GUCY2C activation opposed STBI-induced intestinal dysfunction quantified by body weight loss (FIG. 4C), intestinal bleeding (FIG. 4D) and debilitation (FIG. 4E) in wild type, but not in p53int−/−, mice. These observations suggest that the GUCY2C signaling axis opposes RIGS through a mechanism requiring p53. GUCY2C signaling opposing RIGS is associated with amplification of p53 responses. Consistent with a role for p53 in mediating the effects of GUCY2C signaling on radiation-induced intestinal toxicity, oral ST increased levels of phosphorylated p53 in mouse intestinal epithelial cells in RIGS induced by STBI (FIG. 4F). Recapitulating these in vivo results, the GUCY2C second messenger, cGMP, increased total and phosphorylated p53 induced by radiation in HCT116 human colon carcinoma cells (FIG. 4G), an in vitro model of intestinal epithelial cells that express wild type p53, but not GUCY2C (19,34,35). Amplification of p53 responses to radiation induced by cGMP signaling in HCT116 cells was associated with reduced interactions between p53 and the inhibitory protein Mdm2 (FIG. 4H). GUCY2C signaling opposing RIGS is associated with p53-dependent rescue of mitotic catastrophe. Induction of GUCY2C signaling by oral ST opposed chromosomal instability in intestinal epithelial cells following STBI, reducing double-strand DNA breaks (FIG. 5A) and abnormal mitoses characteristically associated with mitotic catastrophe (FIG. 5B). Similarly, chromosomal instability produced by irradiation, quantified by centrosome counts or the anaphase bridge index (43), was reduced in HCT116 cells treated with 8-Br-cGMP (FIG. 5C-D). In contrast, elimination of p53 (HCT116 p53−/−) amplified chromosomal instability produced by irradiation, and this damage was insensitive to 8-Br-cGMP (FIG. 5C-D). Moreover, cell death by mitotic catastrophe reflecting radiation-induced aberrant mitosis was reduced by cGMP in parental, but not in p53−/−, HCT116 cells (FIG. 5E).

### Discussion

RIGS refers to radiation-induced genotoxic stress in intestinal epithelial cells (7-9,11,12,14). Radiation produces DNA damage, directly and through reactive oxygen species (23), activating p53 (7,9,14,44). In turn, p53 mediates a bifurcated injury response. Cells damaged beyond repair undergo caspase-dependent apoptosis initiated by p53 activation of PUMA (6,9,12,13). Further, in cells that can be rescued, p53 induces the expression of p21, a key inhibitor of cyclin-dependent kinases which regulates cell cycle checkpoints (7,9,14). Inhibition of proliferation associated with these checkpoints permits cells to repair damaged DNA (7-9,12,14,45). However, p53 responses are limited and cells with damaged DNA escape checkpoints within days of irradiation, enter prematurely into the cell cycle with damaged DNA, and undergo mitotic catastrophe (7,9,12,14,46). In turn, this produces epithelial loss and mucositis, disrupting barrier function associated with fluid and electrolyte loss and infection which are principle mechanisms of death from RIGS (47). Here, we reveal an unexpected compensatory mechanism that opposes this pathophysiology, involving the GUCY2C signaling axis at the intersection of radiation injury and p53 responses.

GUCY2C is selectively expressed by intestinal epithelial cells and activation by the endogenous hormones guanylin and uroguanylin, or the diarrheagenic bacterial STs, increases intracellular cGMP accumulation (17). While there is evidence for GUCY2C signaling in other tissues (32,48), the effect of oral ST in ameliorating RIGS in the present study is consistent with a primary effect on intestinal receptors, reflecting the absence of bioavailability of oral GUCY2C ligands (31). GUCY2C-cGMP signaling modulates intestinal secretion, one mechanism by which bacteria induce diarrhea, and the oral GUCY2C ligands linaclotide (Linzess™) and plecanatide (Trulance™) improve constipation and relieve abdominal pain in patients with irritable bowel syndrome (31,49). Further, GUCY2C signaling regulates proliferation and DNA damage repair, processes that are canonically disrupted in RIGS (26). Indeed, signaling through the GUCY2C-cGMP axis inhibits DNA synthesis and prolongs the cell cycle, imposing a G1-S delay in part by regulating p21, key injury responses to radiation (18-20,50). Further, silencing GUCY2C increases DNA oxidation and double strand breaks, amplifying mutations induced by chemical or genetic DNA damage, reflecting ROS and inadequate repair (20). Moreover, silencing GUCY2C disrupts the intestinal barrier (26), a key pathophysiological mechanism contributing to RIGS (47).

Conversely, GUCY2C ligands block that damage, enhancing barrier integrity and accelerating recovery from injury (23,24,26,27,30). This role in promoting mucosal barrier integrity supports GUCY2C as a therapeutic target for RIGS. The present observations suggest a previously unrecognized compensatory mechanism opposing RIGS in which the paracrine hormones guanylin and uroguanylin activate GUCY2CcGMP signaling to defend the integrity of the intestinal epithelial barrier. In that model, paracrine hormone stimulation of the GUCY2C-cGMP signaling axis supports p53 responses to radiation injury by disrupting interactions with Mdm2, a key regulator of responses to genotoxic stress which binds to the amino terminal of 18-19. p53, inhibiting its transactivation function and targeting it for proteasomal degradation (45,51,52). In turn, amplified p53 responses contribute to resolving DNA damage, limiting mitotic catastrophe (7). Beyond these compensatory responses, the durable preservation of GUCY2C expression following high dose irradiation across the rostral-caudal axis of the intestine and the continuum of injury responses offers an opportunity to target this receptor for mitigation of RIGS by oral GUCY2C hormone administration. Indeed, it creates the unique possibility of transforming RIGS from a syndrome of irreversible DNA damage to one that can be reversed or prevented by oral GUCY2C ligand supplementation.

These studies stand in contradistinction to other models of intestinal injury in which homeostasis is disrupted through paracrine hormone loss silencing GUCY2C. Indeed, guanylin and uroguanylin are the most commonly lost gene products in sporadic colorectal cancer and these hormones are lost at the earliest stages of neopasia (29,53,54). Hormone loss silences the GUCY2C signaling axis and interrupts canonical homeostatic mechanisms that regulate the continuously regenerating intestinal epithelium and whose disruption is essential for tumorigenesis (17-20,25,26,34). Similarly, while obesity and colorectal cancer are associated, underlying mechanisms have remained unclear. Recent studies revealed that over-consumption of calories, which is an essential mechanism contributing to obesity, produces ER stress leading to guanylin loss silencing the GUCY2C tumor suppressor (25). Indeed, replacing guanylin suppressed by calories eliminated tumorigenesis (25). Moreover, oral dextran sulfate injures intestinal mucosa, producing inflammatory bowel disease (IBD), and silencing GUCY2C amplifies injury in IBD, increasing mortality in mice (24,26,30). Indeed, IBD is associated with GUCY2C paracrine hormone loss in humans (21). In the context of this emerging paradigm of intestinal epithelial injury, the present results demonstrating the preservation of paracrine hormone expression in the context of high dose irradiation was unexpected. However, they are consistent with a role for the GUCY2C paracrine hormone axis in compensatory mechanisms opposing RIGS. Previous studies revealed that silencing GUCY2C amplified apoptosis induced by low doses of radiation (5 Gy)(22). These radiation doses are below G-toxic levels which produce RIGS or bone marrow failure. Further, silencing GUCY2C (Gucy2c−/− mice) did not alter the induction of apoptosis in small or large intestine in RIGS, in contrast to those earlier studies (see FIG. 4A). Moreover, silencing GUCY2C recapitulated the effects of eliminating p53 signaling (p53−/− mice), which also had no effect on apoptosis in small or large intestine in RIGS as reported previously (7). In the context of the role of GUCY2C in optimizing p53 injury responses, reinforced by the ability of Gucy2c−/− mice to phenocopy p53−/− mice (7), the primary mechanism amplifying epithelial disruption in RIGS in the absence of GUCY2C appears to be mitotic catastrophe, rather than apoptosis. Targeting p53 directly to prevent and treat RIGS has been uniquely challenging and treatments exploiting this mechanism have not yet emerged (7,9,12,14). The therapeutic challenge arises from the paradoxically opposite roles of p53 in RIGS and the radiation-induced hematopoietic syndrome, the two principal toxicities associated with radiation. Protection of epithelial cells by p53 has made its activation a target to treat RIGS (7-9,14,46). In striking contrast, radiation toxicity mediated by p53 in bone marrow has made its inhibition a target to treat the hematopoietic syndrome (8,46,55). Hence, p53 has remained an elusive target, requiring tissue specific strategies for appropriate directional regulation. The present study provides insights into novel molecular mechanisms underlying the pathophysiology of RIGS that can be readily translated into p53-targeted medical countermeasures to prevent and treat acute radiation induced GI toxicity. Thus, GUCY2C has a narrow tissue distribution, selectively expressed by intestinal epithelial cells from the duodenum to the rectum (15-17). Further, GUCY2C is anatomically privileged, expressed in lumenal membranes of those cells, directly accessible to oral agents but inaccessible to the systemic compartment (17,31). Moreover, linaclotide (Linzess™) and plecanatide (Trulance™) are oral GUCY2C ligands recently approved for the treatment of chronic constipation syndromes, with negligible oral bioavailability or bioactivity outside the GI tract (31). The anatomical privilege of GUCY2C coupled with the compartmentalized activity of linaclotide and plecanatide, confined only to the intestinal lumen, offers a uniquely targeted approach to specifically engage p53-dependent mechanisms, compared to other available approaches, to prevent and treat RIGS. In turn, this offers prophylactic and therapeutic solutions to civilians, first-responders, and military personnel at risk from radiation disasters, like Chernobyl or Fukushima. Similarly, it provides a clinically tractable approach for targeted prevention of GI toxicity from abdominopelvic radiotherapy for cancer, reducing dose-limiting toxicities and permitting greater radiation fractions to be administered, without altering the therapeutic radiosensitivity of extra-intestinal tumors (see FIG. 3I)(5).

### REFERENCES (WHICH ARE HEREBY INCORPORATED HEREIN BY REFERENCE)

1. Terry N H, Travis E L. The influence of bone marrow depletion on intestinal radiation damage. Int J Radiat Oncol Biol Phys 1989; 17:569-73
2. Mason K A, Withers H R, Davis C A. Dose dependent latency of fatal gastrointestinal and bone marrow syndromes. Int J Radiat Biol 1989; 55:1-5
3. Mason K A, Withers H R, McBride W H, Davis C A, Smathers J B. Comparison of the gastrointestinal syndrome after total-body or total-abdominal irradiation. Radiat Res 1989; 117:480-8
4. Berger M E, Christensen D M, Lowry P C, Jones O W, Wiley A L. Medical management of radiation injuries: current approaches. Occup Med (Lond) 2006; 56:162-72
5. Rodriguez M L, Martin M M, Padellano L C, Palomo A M, Puebla Y I. Gastrointestinal toxicity associated to radiation therapy. Clin Transl Oncol 2010; 12:554-61
6. Jeffers J R, Parganas E, Lee Y, Yang C, Wang J, Brennan J, et al. Puma is an essential mediator of p53-dependent and -independent apoptotic pathways. Cancer Cell 2003; 4:321-8
7. Kirsch D G, Santiago P M, di Tomaso E, Sullivan J M, Hou W S, Dayton T, et al. p53 controls radiation-induced gastrointestinal syndrome in mice independent of apoptosis. Science 2010; 327:593-6
8. Komarova E A, Kondratov R V, Wang K, Christov K, Golovkina T V, Goldblum J R. of al. Dual effect of p53 on radiation sensitivity in vivo: p53 promotes hematopoietic injury, but protects from gastro-intestinal syndrome in mice. Oncogene 2004; 23:3265-71
9. Leibowitz B J, Qiu W, Liu H, Cheng T, Zhang L, Yu J. Uncoupling p53 functions in adiation induced intestinal damage via PUMA and p21. Molecular cancer research: MCR 2011; 9:616-25
10. Paris F, Fuks Z, Kang A, Capodieci P, Juan G, Ehleiter D, et al. Endothelial apoptosis as the primary lesion initiating intestinal radiation damage in mice. Science 2001; 293:293-7
11. Potten C S. Radiation, the ideal cytotoxic agent for studying the cell biology of tissues such as the small intestine. Radiat Res 2004; 161:123-36
12. Qiu W, Carson-Walter E B, Liu H, Epperly M, Greenberger J S, Zambetti G P, et al. PUMA regulates intestinal progenitor cell radiosensitivity and gastrointestinal syndrome. Cell Stem Cell 2008; 2:576-83
13. Villunger A, Michalak E M, Coultas L, Mullauer F, Bock G, Ausserlechner M J, et al. p53-and drug-induced apoptotic responses mediated by BH3-only proteins puma and noxa. Science 2003; 302:1036-8
14. Sullivan J M, Jeffords L B, Lee C L, Rodrigues R, Ma Y, Kirsch D G. p21 protects “Super p53” mice from the radiation-induced gastrointestinal syndrome. Radiat Res 2012; 177:307-10
15. Schulz S, Green C K, Yuen P S, Garbers D L. Guanylyl cyclase is a heat-stable enterotoxin receptor. Cell 1990; 63:941-8
16. Schulz S, Lopez M J, Kuhn M, Garbers D L. Disruption of the guanylyl cyclase-C gene leads to a paradoxical phenotype of viable but heat-stable enterotoxin-resistant mice. J Clin Invest 1997; 100:1590-5
17. Kuhn M. Molecular physiology of membrane guanylyl cyclase receptors. Physiol Rev 2016; 96:751-804
18. Li P, Lin J E, Chervoneva 1, Schulz S, Waldman S A, Pitari G M. Homeostatic control of the crypt-villus axis by the bacterial enterotoxin receptor guanylyl cyclase C restricts the proliferating compartment in intestine. Am J Pathol 2007; 171:1847-58
19. Li P, Schulz S, Bombonati A, Palazzo J P, Hyslop T M, Xu Y. et al. Guanylyl cyclase C suppresses intestinal tumorigenesis by restricting proliferation and maintaining genomic integrity. Gastroenterology 2007; 133:599-607
20. Lin J E, Li P, Snook A E, Schulz S, Dasgupta A, Hyslop T M, et al. The hormone receptor GUCY2C suppresses intestinal tumor formation by inhibiting AKT signaling. Gastroenterology 2010; 138:241-54
21. Brenna O, Bruland T, Fumes M W, Granlund A, Drozdov I, Emgard J, et al. The guanylate cyclase-C signaling pathway is down-regulated in inflammatory bowel disease. Scand J Gastroenterol 2015:50:1241-52
22. Garin-Laflam M P, Steinbrecher K A, Rudolph J A, Mao J, Cohen M B. Activation of guanylate cyclase C signaling pathway protects intestinal epithelial cells from acute radiation-induced apoptosis. Am J Physiol Gastrointest Liver Physiol 2009; 296:G740-9
23. Han X, Mann E, Gilbert S, Guan Y, Steinbrecher K A, Montrose M H, et al. Loss of guanylyl cyclase C (GCC) signaling leads to dysfunctional intestinal barrier. PLoS One 2011; 6:e16139
24. Flannel-Laws E, Mann E A, Cohen M B, Steinbrecher K A. Guanylate cyclase C deficiency causes severe inflammation in a murine model of spontaneous colitis. PLoS One 2013; 8:e79180
25. Lin J E, Colon-Gonzalez F, Blomain E, Kim G W, Aing A, Stoecker B, et al. Obesity-induced colorectal cancer is driven by caloric silencing of the guanylin-GUCY2C paracrine signaling axis. Cancer Res 2016; 76:339-46
26. Lin J E, Snook A E, Li P, Stoecker B A, Kim G W, Magee M S, et al. GUCY2C opposes systemic genotoxic tumorigenesis by regulating AKT-dependent intestinal barrier integrity. PLoS One 2012; 7:e31686
27. Mann E A, Harmel-Laws E, Cohen M B, Steinbrecher K A. Guanylate cyclase C limits systemic dissemination of a murine enteric pathogen. BMC Gastroenterol 2013:13:135
28. Shailubhai K, Palejwala V, Arunan K P, Saykhedkar S, Nefsky B, Foss J A, et al. Plecanatide and dolcanatide, novel guanylate cyclase-C agonists, ameliorate gastrointestinal inflammation in experimental models of murine colitis. World J Gastrointest Pharmacol Ther 2015; 6:213-22
29. Shailubhai K, Yu H H, Karunanandaa K, Wang J Y, Eber S L, Wang Y, et al. Uroguanylin treatment suppresses polyp formation in the Apc(Min/+) mouse and induces apoptosis in human colon adenocarcinoma cells via cyclic GMP. Cancer Res 2000; 60:5151-7
30. Steinbrecher K A, Harmel-Laws E, Garin-Laflam M P, Mann E A, Bezerra L D, Hogan S P, et al. Murine guanylate cyclase C regulates colonic injury and inflammation. J Immunol 2011; 186:7205-14
31. Camilleri M. Guanylate cyclase C agonists: emerging gastrointestinal therapies and actions. Gastroenterology 2015; 148:483-7
32. Valentino M A, Lin J E, Snook A E, Li P, Kim G W, Marszlowicz G, et al. A uroguanylin-GUCY2C endocrine axis regulates feeding in mice. J Clin Invest 2011; 121:3578-88
33. Waldman S A, Barber M, Pearlman J, Park J, George R, Parkinson S J. Heterogeneity of guanylyl cyclase C expressed by human colorectal cancer cell lines in vitro. Cancer Epidemiol Biomarkers Prev 1998; 7:505-14
34. Gibbons A V, Lin J E, Kim G W, Marszalowicz G P, Li P, Stoecker B A, et al. Intestinal GUCY2C prevents TGF-beta secretion coordinating desmoplasia and hyperproliferation in colorectal cancer. Cancer Res 2013; 73:6654-66
35. Zuzga D S, Pelta-Heller J, Li P, Bombonati A, Waldman S A, Pitari G M. Phosphorylation of vasodilator-stimulated phosphoprotein Ser239 suppresses filopodia and invadopodia in colon cancer. Int J Cancer 2012; 130:2539-48
36. Bunz F, Dutriaux A, Lengauer C, Waldman T, Zhou S, Brown J P, et al. Requirement for p53 and p21 to sustain G2 arrest after DNA damage. Science 1998; 282:1497-501
37. Li Z. Taylor-Blake B, Light A R, Goy M F. Guanylin an endogenous ligand for C-type guanylate cyclase, is produced by goblet cells in the rat intestine. Gastroenterology 1995; 109:1863-75
38. Perkins A, Goy M F, Li Z. Uroguanylin is expressed by enterochromaffin cells in the rat gastrointestinal tract. Gastroenterology 1997; 113:1007-14
39. Faget L, Hnasko T S. Tyramide signal amplification for immunofluorescent enhancement. Methods Mol Biol 2015; 1318:161-72
40. Moreau M R, Wijetunge D S, Bailey M L, Gongati S R, Goodfield L L, Hewage E M, et al Growth in egg yolk enhances Salmonella enteritidis colonization and virulence in a mouse model of human colitis. PLoS One 2016; 11:e0150258
41. Fierer J, Okamoto S, Banerjee A, Guiney D G. Diarrhea and colitis in mice require the Salmonella pathogenicity island 2-encoded secretion function but not SifA or Spv effectors. Infect Immun 2012; 80:3360-70
42. Brenna O, Fumes M W, Munkvold B, Kidd M, Sandvik A K, Gustafsson B. Cellular localization of guanylin and uroguanylin mRNAs in human and rat duodenal and colonic mucosa. Cell Tissue Res 2016; 365:331-41
43. Ganem N J, Godinho S A, Pelman D. A mechanism linking extra centrosomes to chromosomal instability. Nature 2009; 460:278-82
44. Marusyk A, Casas-Selves M, Henry C J, Zaberezhnyy V, Klawitter J, Christians U, et al. Irradiation alters selection for oncogenic mutations in hematopoietic progenitors. Cancer Res 2009; 69:7262-9
45. Vousden K H, Prives C. Blinded by the Light: The growing complexity of p53. Cell 2009; 137:413-31
46. Lee C L, Blum J M, Kirsch D G. Role of p53 in regulating tissue response to radiation by mechanisms independent of apoptosis. Translational cancer research 2013; 2:412-21
47. Hauer-Jensen M, Denham J W, Andreyev H J. Radiation enteropathy—pathogenesis, treatment and prevention. Nature reviews Gastroenterology & hepatology 2014; 11:470-9
48. Carrithers S L, Hill M J, Johnson B R, O'Hara S M, Jackson B A, Ott C E, et al. Renal effects of uroguanylin and guanylin in vivo. Brazilian journal of medical and biological research=Revista brasileira de pesquisas medicas e biologicas 1999; 32:1337-44
49. Castro J, Harrington A M, Hughes P A, Martin C M, Ge P, Shea C M, et al. Linaclotide inhibits colonic nociceptors and relieves abdominal pain via guanylate cycase-C and extracelular cyclic guanosine 3′,5′-monophosphate. Gastroenterology 2013; 145:1334-46 el-11
50. Basu N, Saha S, Khan I, Ramachandra S G, Visweswariah S S. Intestinal cell proliferation and senescence are regulated by receptor guanylyl cyclase C and p21. The Journal of biological chemistry 2014; 289:581-93
51. Chene P. Inhibiting the p53-MDM2 interaction: an important target for cancer therapy. Nature reviews Cancer 2003; 3:102-9
52. Prives C. Signaling to p53: breaking the MDM2-p53 circuit. Cell 1998; 95:5-8
53. Wilson C, Lin J E, Li P, Snook A E, Gong J, Sato T, et al. The paracrine hormone for the GUCY2C tumor suppressor, guanylin, is universally lost in colorectal cancer. Cancer Epidemiol Biomarkers Prev 2014; 23:2328-37
54. Steinbrecher K A, Tuohy T M, Heppner Goss K, Scott M C, Witte D P, Groden J, et al. Expression of guanylin is downregulated in mouse and human intestinal adenomas. Biochem Biophys Res Commun 2000; 273:225-30
55. Lotem J, Sachs L. Hematopoietic cells from mice deficient in wild-type p53 are more resistant to induction of apoptosis by some agents. Blood 1993; 82:1092-6

### Example 2

Long-lived multipotent stem cells (ISCs) at the base of intestinal crypts adjust their phenotypes to accommodate normal maintenance and post-injury regeneration of the epithelium. Their long life, lineage plasticity, and proliferative potential underlie the necessity for tight homeostatic regulation of the ISC compartment. In that context, the guanylate cycase C (GUCY2C) receptor and its paracrine ligands regulate intestinal epithelial homeostasis, including proliferation, lineage commitment, and DNA damage repair. However, a role for this axis in maintaining ISCs remains unknown. Transgenic mice enabling analysis of ISCs (Lgr5-GFP) in the context of GUCY2C elimination (Gucy2c−/−) were combined with immunodetection techniques and pharmacological treatments to define the role of the GUCY2C signaling axis in supporting ISCs. ISCs were reduced in Gucy2c−/− mice, associated with loss of active Lgr5+ cells but a reciprocal increase in reserve Bmi1+ cells. GUCY2C was expressed in crypt base Lgr5+ cells in which it mediates canonical cyclic (c)GMP-dependent signaling. Endoplasmic reticulum (ER) stress, typically absent from ISCs, was elevated throughout the crypt base in Gucy2c−/− mice. The chemical chaperone tauroursodeoxycholic acid resolved this ER stress and restored the balance of ISCs, an effect mimicked by the GUCY2C effector 8Br-cGMP. Reduced ISCs in Gucy2c−/− mice was associated with greater epithelial injury and impaired regeneration following sub-lethal doses of irradiation. These observations suggest that GUCY2C provides homeostatic signals that modulate ER stress and cell vulnerability as part of the machinery contributing to the integrity of ISCs.

Introduction

The intestinal epithelium is highly dynamic, undergoing continuous cycles of renewal and repair. Stem cells at the base of crypts give rise to progenitor cells that continue to divide, migrate up the crypt-villus axis, and differentiate into the specialized epithelial cell types of the intestine [56]. Absorptive cells are sloughed off into the intestinal lumen in a conveyor belt fashion on a weekly basis, while secretory cells such as tuft cells and Paneth cells survive for weeks [57, 58]. Beyond this programmed turnover, intestinal insults, such as inflammation, oxidative damage, and radiation [59, 60] induce cell death, requiring replacement to maintain the epithelial barrier. These processes of turnover and regeneration are driven by an equally dynamic population of intestinal stem cells (ISCs) whose characteristics are only beginning to emerge [57].

The highly organized ISC compartment at the base of crypts contains cell types with distinct marker expression and functional phenotypes. Lgr5+, or crypt base columnar (CBC), cells are long-lived multipotent stem cells located at crypt cell positions 0-4 that divide daily to drive weekly turnover of the epithelium, making them the “active” stem cells [61]. These cells are exquisitely sensitive to insult and are intimately associated with differentiated cells that supply essential regulatory signals, including Paneth cells [61-63]. Another long-lived, multipotent stem cell type located higher up the crypt axis around cell positions 4-8 commonly expresses the marker Bmi1 [64]. These Bmi1+ cells are quiescent and contribute minimally to tissue homeostasis [61]. However, upon injury, Bmi1+ cells can restore both the more active CBCs as well as all of the differentiated cell types of the intestinal epithelium, earning them the label of “reserve” ISC [61, 65]. Despite the sensitivity of Lgr5+ cells to death upon intestinal insult and the contribution of Bmi1+ cells to regeneration, Lgr5+ cells are required for recovery from radiation-induced gastrointestinal damage [60]. While the identity and function of intestinal stem cell populations are emerging, mechanisms contributing to their maintenance and relative balance continue to be refined [61-63, 66].

GUCY2C is a membrane-associated guanylate cyclase receptor selectively expressed in apical membranes of intestinal epithelial cells from the duodenum to the distal rectum [67]. Cognate ligands are structurally similar peptides and include the paracrine hormones guanylin, produced throughout the intestine, and uroguanylin, produced selectively in small intestine, and the heat-stable enterotoxins (STs) produced by diarrheagenic bacteria [67]. GUCY2C originally was identified as a mediator of intestinal fluid and electrolyte secretion contributing to the pathophysiology of enterotoxigenic diarrhea [67]. However, the GUCY2C-paracrine hormone axis has emerged as an essential regulator of key homeostatic processes, including cell proliferation [68, 69], lineage commitment [70], and DNA damage repair [69], functions that are essential to the integrity of the crypt [71]. Further, in murine models of tumorigenesis or inflammatory bowel disease, in which injury and recovery characteristically involve ISCs [72], silencing GUCY2C amplifies pathophysiology, tissue damage, and mortality [69, 73-76]. Here, we explore the role for GUCY2C signaling in maintaining ISCs.

### Results

### Eliminating GUCY2C Expression Disrupts ISC Numbers

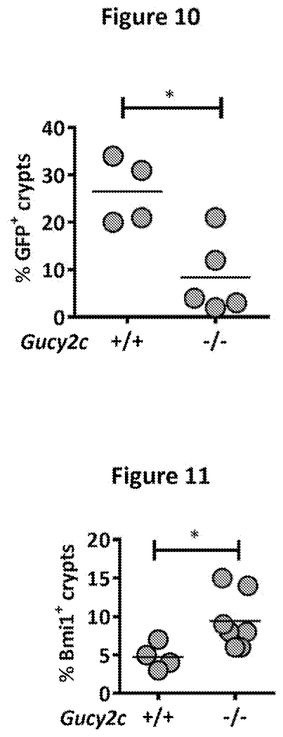

Stem cells were enumerated in small intestinal crypts from Gucy2c^{+/+} and Gucy2c^{-/-} mice by electron microscopy. Wedge-shaped cells in crypt positions 0 to 5 were included, and Paneth cells were excluded by their vesicular morphology (FIG. 6 panel A) [61-63]. The total number of ISCs in the crypt base was reduced in the absence of GUCY2C (FIG. 6 panel B). Similarly, ex vivo enteroid formation, a measure of ISC number and function [77], was reduced in the absence of GUCY2C (p<0.001; FIG. 6 panel C). FACS analyses revealed fewer Lgr5⁺/GFP^{High} cells in Lgr5-EGFP-IRES-CreERT2 mice in which GUCY2C was eliminated (Lgr5-EGF-Cre-Gucy2c^{-/-}; FIG. 6 panel D), confirmed by immunofluorescence microscopy (FIG. 6 panels E-F; FIG. 10). Moreover, lineage tracing in Lgr5-EGFP-Cre-Gucy2c^{+/+} and −Gucy2c^{-/-} mice crossed onto the Rosa-STOP^{fl}-LacZ background revealed that Gucy2c^{-/-} mice had fewer LacZ-labeled crypts (FIG. 6 panels G-H). Conversely, Gucy2c^{-/-} mice exhibited an expanded population of Bmi1⁺ cells by immunofluorescence microscopy (FIG. 6 panels I-J; FIG. 11) which was confirmed by immunoblot analysis (FIG. 6 panels K-L). Together, these results suggest that eliminating GUCY2C signaling rebalances stem cell populations, favoring a “reserve” ISC phenotype.

### Functional Expression of the GUCY2C Signaling Axis in ISCs

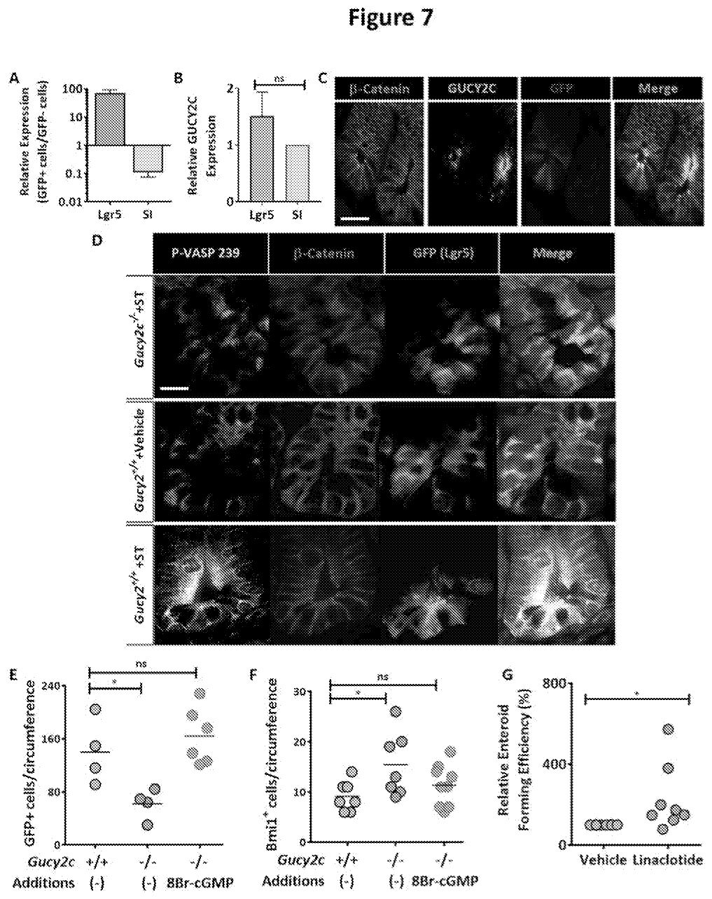

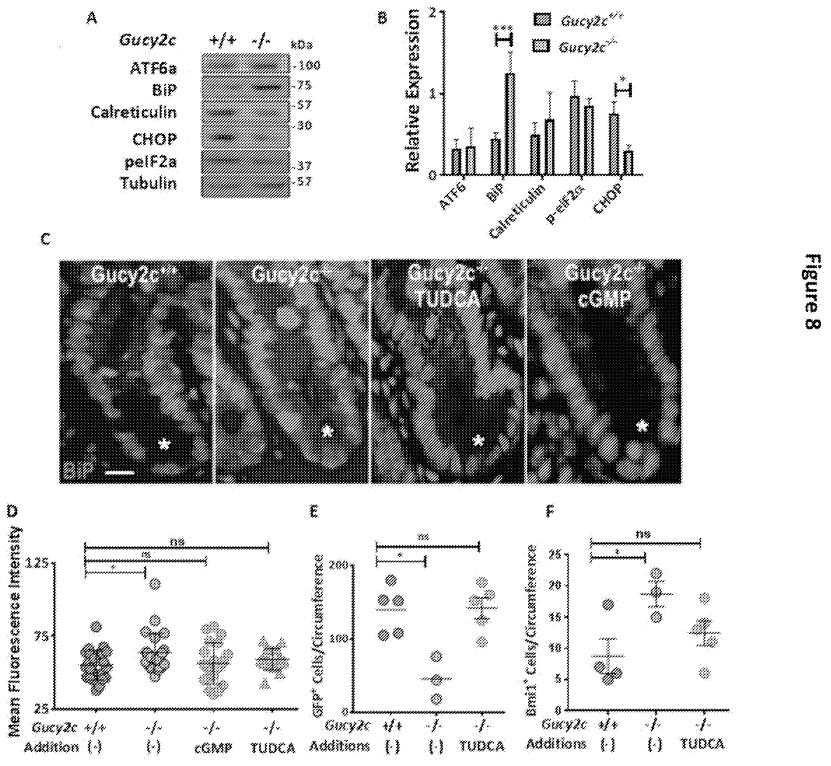

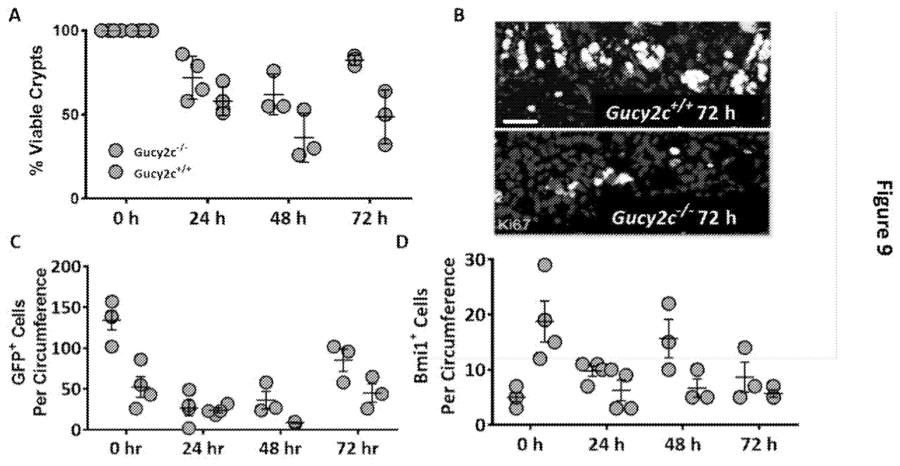

Lgr5⁺GFP⁺ cells were collected by FACS from Lgr5-EGFP-Cre-Gucy2c^{+/+} and −Gucy2c^{-/-} mice [78] and enrichment verified by RT-qPCR of stem (Lgr5) and differentiated cell [sucrose isomaltase (SI)] mRNA markers (FIG. 7 panel A). Expression of Gucy2c mRNA in stem (Lgr5^{High}/SI^{Low}) cells was quantitatively similar to that of differentiated (Lgr5^{Low}/SI^{High}) cells suggesting similar levels of expression in stem cell and differentiated compartments (FIG. 7 panel B). Immunofluorescence microscopy confirmed specific co-localization of GUCY2C in Lgr5⁺GFP⁺ stem cells (FIG. 7 panel C). To confirm functionality of the GUCY2C receptor in ISCs, ST was injected into segments of intestinal lumen of Lgr5-EGFP-Cre-Gucy2c^{+/+} and Lgr5-EGFP-Cre-Gucy2c^{-/-} mice [79]. Luminal exposure to this GUCY2C agonist [80] produced cGMP accumulation and cGMP-specific phosphorylation of the downstream target of cGMP-dependent protein kinase, vasodilator-stimulated phosphoprotein (VASP), in Lgr5⁺GFP⁺ cells, in Gucy2c^{+/+}, but not in Gucy2c^{-/-}, mice (FIG. 7 panel D) highlighting the functionality of GUCY2C in ISCs. Further, 8Br-cGMP, a cell-permeable analog of the GUCY2C second messenger cGMP [81], restored the balance of ISCs, returning Lgr5⁺GFP⁺ (FIG. 7 panel E) and Bmi1⁺ (FIG. 7 panel F) cells in Gucy2c^{-/-} mice to levels that were comparable to those in Gucy2c^{+/+} mice. Moreover, the oral GUCY2C agonist linaclotide (Linzess™ Ironwood, Cambridge, Mass.) amplified the efficiency of enteroid formation in Gucy2c^{+/+} mice (FIG. 7 panel G). These observations reinforce the role of GUCY2C signaling in maintaining ISCs.

### GUCY2C Signaling Opposes Crypt ER Stress

The normal ISC compartment minimizes endoplasmic reticulum (ER) stress, and prolonged exposure induces ISCs to shift from the stem cell compartment into the proliferating progenitor cell pool [82, 83], an effect which is phenocopied by eliminating GUCY2C signaling [68-70, 75, 84]. Here, elimination of GUCY2C expression induced over-expression of the chaperone protein BiP (Grp78), a canonical marker of ER stress [85], in crypts in Gucy2c^{-/-} mice (FIG. 8 panels A-D). Interestingly, markers of the unfolded protein response induced by ER stress, including ATF6, caireticulin, and phosphorylated eIF2α (p-eIF2α), were unchanged in those crypts [86] (FIG. 8 panels A, B). Moreover, the pro-apoptotic protein CHOP, which eliminates cells with irreversible ER stress [87], was paradoxically reduced in those crypts (FIG. 8 panels A, B). This pattern of markers specifically reflects adaptive ER stress, in which chaperones like BiP are over-expressed to relieve chronic ER stress, minimizing the unfolded protein response, while CHOP transcription is down-regulated to prevent cell death [88, 89]. In that context, tauroursodeoxycholic acid (TUDCA), a bile salt that mimics the chaperone protein BiP to reduce ER stress by relieving protein misfolding [90], restored normal BiP expression in crypts in Gucy2c^{-/-} mice, an effect which was mimicked by 8Br-cGMP (FIG. 8 panels C-D). Moreover, like 8Br-cGMP (FIG. 7 panels F-G), TUDCA also restored Lgr5⁺GFP⁺ (FIG. 8 panel E) and Bmi1⁺ (FIG. 8 panel F) cells to normal levels in Gucy2c^{-/-} mice. These observations underscore the role of GUCY2C signaling in opposing ER stress that is essential to maintaining ISCs.

GUCY2C Maintains ISCs Supporting Regeneration after Radiation Injury

Intestinal irradiation is an established model to quantify ISC vulnerability and regenerative capacity [91]. Lgr5⁺ cells are exquisitely sensitive to, and depleted by, irradiation while Bmi1⁺ cells are recruited to expand and repopulate the crypt base to support regeneration [61]. A single sub-lethal 10 Gy dose of whole-body radiation produced massive crypt death quantified by the microcolony assay [92] in small intestines of Gucy2c^{+/+} and Gucy2c^{-/-} mice (FIG. 4A). However, Gucy2c^{-/-} mice displayed greater fractional crypt loss compared to Gucy2c^{+/+} mice 48 hours after irradiation (36% vs 62%, p<0.05), consistent with increased susceptibility to radiation-induced ISC cell death in the absence of GUCY2C signaling (FIG. 9 panel A). Further, the absence of GUCY2C signaling was associated with a regenerative lag; Gucy2c^{+/+} mice recovered only 49% of their crypts compared to 82% in Gucy2c^{+/+} mice at 72 hours (p<0.01), consistent with enhanced vulnerability of the crypt in the absence of GUCY2C (FIG. 9 panels A-B), Indeed, the absolute number of Lgr5⁺GFP⁺ cells 48 hours after irradiation was lower (31 vs 9, p<0.05) in Gucy2c^{-/-}, compared to Gucy2c^{+/+}, mice (FIG. 9 panel C). In contrast, Bmi1⁺ cells expanded to repopulate the crypt after radiation in Gucy2c^{+/+} mice, achieving a maximum response at 48 h, while in Gucy2c^{-/-} mice there was a paradoxical loss of those cells, without recovery, (p<0.01; FIG. 9 panel D), paralleling the regenerative lag (FIG. 9 panel A). Together, these observations support the hypothesis that GUCY2C signaling, at least in part, protects Lgr5⁺ and Bmi1⁺ stem cells required for regenerative responses to radiation injury.

### Discussion

An emerging paradigm suggests that the crypt harbors populations of multi-potent stem cells which support the unique homeostatic requirements of the continuously regenerating intestinal epithelium. While several intestinal stem cell populations have been suggested, reflecting phenotypic and functional characteristics, there is consensus on two broad categories [93]. Active crypt base stem cells at position 0-4 which are rapidly proliferating and sensitive to insults like radiation are the source of transit amplifying cells which ultimately replace differentiated epithelial cells in routine mucosal maintenance [61, 94]. In contrast, stem cells residing at positions above 4, which are slowly proliferating and relatively resistant to insults, comprise a reserve population that regenerates the intestinal epithelium following injury [95]. While several protein markers have been purported to identify discreet stem cell populations, all are variably expressed by ISCs in crypts [96]. However, Lgr5 and Bmi1 appear to be relatively selective as markers of active and reserve stem cell populations, respectively [93]. This heterogeneity in marker expression likely reflects the plasticity of ISCs. Indeed, rather than discreet stable populations, ISCs likely transition between active and reserve phenotypes to meet the instantaneous needs of normal or injured epithelium [97]. This plasticity creates functional capacity to accommodate wide variations in environmental challenges to the integrity of the mucosa [98]. In turn, this plasticity requires specific mechanisms that maintain the quantity and relative balance of active and reserve stem cells and are only now being discovered.

Here, we reveal that GUCY2C is one key determinant of the quantity and relative balance of active and reserve ISCs. In the absence of GUCY2C, there is a reduction in the quantity of ISCs, reflected in their overall number and in their ability to form enteroids ex vivo. Also, there is a shift in the relative balance of these cells with a decrease in active Lgr5⁺ cells and a reciprocal increase in reserve Bmi1⁺ cells. Regulation of the quantity and relative balance of ISCs is associated with the functional co-expression of GUCY2C in stem cells. In that context, reconstitution of cGMP signaling by oral delivery of 8Br-cGMP in Gucy2c^{-/-} mice restored the quantity and relative balance of active and reserve stem cells. Eliminating GUCY2C is associated with chronic ER stress in crypts, a process associated with loss of stem cells in intestine [89, 99]. ER stress may contribute to ISC loss in Gucy2c^{-/-} mice since 8Br-cGMP or TUDCA, a chemical chaperone [90], resolved ER stress and restored the quantity and balance of Lgr5⁺ and Bmi1⁺ stem cells. Importantly, silencing GUCY2C increased ISC vulnerability, stem cell loss, and epithelial injury and delayed regeneration in Gucy2c^{-/-} mice exposed to sub-lethal doses of radiation. These observations highlight a previously unknown role for GUCY2C in maintaining and balancing pools of active and reserve stem cells which, in turn, impacts regenerative epithelial responses to environmental insults.

Mechanisms regulating ISC pools by GUCY2C are likely complex and multi-factorial. Generally, GUCY2C effects are mediated by luminocentric paracrine and autocrine signaling driven by the hormones guanylin and uroguanylin [74]. In ISCs, this regulation may be mediated selectively by guanylin, whose mRNA is expressed in intestinal crypts [100]. The effects of hormone signaling may be cell-autonomous, mediated directly by ISCs, which express GUCY2C in apical membranes making them accessible to luminocentric hormone secretion. Alternatively, these effects may be non-autonomous reflecting the essential role of Paneth cells in maintaining ISCs [57, 63, 78, 91] and the loss of those cells when GUCY2C is silenced [69]. Also, loss of ISCs in the absence of GUCY2C may reflect the associated ER stress, which exits stem cells out of the active Lgr5⁺ pool and into the proliferating progenitor (transit amplifying) pool as part of the canonical differentiation program that renews the intestinal epithelium [89]. Indeed, these observations provide one mechanistic explanation for expansion of the proliferating progenitor cell compartment in intestinal crypts in Gucy2c^{-/-} mice [68-70, 75, 84]. Further, loss of ISCs in the absence of GUCY2C may reflect an increase in stem cell vulnerability to environmental insults, again likely reflecting the associated chronic ER stress which amplifies stem cell susceptibility to apoptosis [99]. In that regard, GUCY2C signaling enhances resistance of intestinal epithelial cells to chemical, inflammatory and radiation-induced injury [69, 73, 76, 101-103]. Moreover, here we reveal that active Lgr5⁺ cells and reserve Bmi1⁺ cells, which are typically resistant to insults [61], are sensitized to radiation injury in the absence of GUCY2C signaling. Beyond exiting stem cells from the ISC pool and amplifying their vulnerability, the impact of GUCY2C signaling on the plasticity of ISCs and their ability to shift between active and reserve pools remains to be defined. In that context, while there is a reciprocal increase in the reserve Bmi1⁺ cell pool in Gucy2c^{-/-} mice, these cells fail to fully compensate for the loss of, or restore, active Lgr5⁺ cells in the normal or irradiated epithelium, respectively. These observations suggest that GUCY2C signaling may play a role in the interconversion of Bmi1⁺ and Lgr5⁺ cells that, in part, defines the functional capacity to regenerate in response to environmental insults.

Based on the present observations, it is tempting to speculate that the role of GUCY2C signaling in pathophysiological mechanisms reflects, at least in part, a contribution of dysregulation of the ISC compartment. The GUCY2C signaling axis is universally silenced in colorectal cancer reflecting loss of expression of guanylin in transforming crypts [104-106]. Conversely, eliminating GUCY2C expression promotes intestinal tumorigenesis [69, 75, 107]. The current pathophysiological paradigm of intestinal cancer suggests that initiating transformational events occur in the stem cell compartment [108]. Further, Bmi1 has been identified as an important transcription factor supporting the transformation of cancer stem cells in a variety of tumors [109, 110]. Moreover, GUCY2C is a key component of mechanisms regulating DNA damage repair [69]. These observations suggest a hypothesis in which loss of guanylin silences GUCY2C, shifting ISC pools from active Lgr5⁺ cells to Bmi1⁺ cells which, in the absence of cGMP signaling, may be particularly vulnerable to genotoxic insults amplifying the risk of transformation and cancer. Similarly, inflammatory bowel disease (IBD) is associated with a loss of components of the GUCY2C signaling axis [111]. Conversely, eliminating GUCY2C signaling amplifies tissue injury and mortality in rodent models of IBD [73, 76, 102, 103]. These data suggest a hypothesis in which the loss of GUCY2C signaling in IBD changes the quantity, balance, and quality of stem cells which, in turn, contributes to their vulnerability to injury and attenuates regenerative responses restoring the damaged epithelium. These considerations suggest previously unanticipated pathophysiological paradigms underlying colorectal cancer and IBD which can be explored in future studies.

Beyond pathophysiology, these observations suggest correlative translational opportunities to develop novel therapeutic and preventive approaches that target ISCs. In that context, there are several oral GUCY2C ligands approved, or in development, to treat chronic constipation syndromes [112]. Lumenal expression of GUCY2C by stem cells highlights the feasibility of targeting this receptor using oral replacement strategies to correct paracrine hormone insufficiencies creating dysfunction in the ISC compartment. Indeed, here the FDA-approved oral GUCY2C ligand linaclotide (Linzess™) amplified the enteroid-forming capacity, a measure of stem cell quantity and quality, in wild type mice (see FIG. 7 panel H). Further, lumenal GUCY2C ligand replacement attenuates intestinal tumorigenesis in mice, and oral GUCY2C ligands are being examined as a novel chemoprevention strategy for colorectal cancer in humans [107, 113, 114]. Additionally, lumenal replacement of GUCY2C ligands ameliorates inflammation in mice, and these agents are in early clinical development in IBD patients [76, 115]. Moreover, the present study reveals that silencing the GUCY2C axis exacerbates the radiation-induced gastrointestinal syndrome (RIGS), the pathophysiology of which has been largely attributed to damage and death of ISCs [116, 117]. The present observations underscore the potential for therapeutic targeting of this signaling axis using oral GUCY2C ligands to defend the crypt to attenuate or prevent RIGS.

In conclusion, we demonstrate that the guanylate cyclase C (GUCY2C) paracrine signaling axis, a key regulator of intestinal epithelial homeostasis, maintains the integrity and balance of active and reserve intestinal stem cells by modulating endoplasmic reticulum stress. These studies reveal a novel role for GUCY2C in supporting intestinal stem cells, Importantly, they underscore the therapeutic potential of oral GUCY2C ligands to prevent or treat diseases reflecting intestinal stem cell dysfunction, including the radiation-induced gastrointestinal syndrome.

### Materials and Methods

### Mice and Treatments

Gucyc^{-/-} (Glucy2^{ctm1Gar}[63]), Lgr5-EGFP-CreERT2 (B6.129P2-Lgr5^{tm1(cre/ERT2)Cle}/J; Jax, Bar Harbor, Me., #008875) and Rosa-STOP^{fl}-LacZ (B6.129S4-Gt(ROSA)26Sor^{tm1Sor}/J; Jax #003474) transgenic mouse lines were interbred to generate offspring with the desired alleles. All mice were co-housed and Gucy2c^{+/+} (wild type) littermates with the appropriate alleles were used as controls. Tissues were harvested from adult mice (12-16 wk of age). Cre was induced with a single 200 μL dose of tamoxifen (Sigma; Billerica, Mass.; T5648) in sunflower oil at 10 mg/ml. Tauroursodeoxycholic Acid (TUDCA, Millipore 580549) treatments were administered daily for 3 d at 100 mg/kg/day intraperitoneally. Mice were exposed to a single 10 Gy dose of whole-body γ-irradiation with a PanTak, 310 kVe x-ray machine and tissues were harvested at noted time points after irradiation. In some experiments, mice were gavaged daily for 7 d with 100 μL of 20 mM 8-cpt-cGMP. Each point in a graph (n) represents one mouse unless otherwise noted. All animal protocols were approved by the Thomas Jefferson University Institutional

### Animal Care and Use Committee.

### Immunohistochemistry and Immunofluorescence

Intestines were harvested from mice, fixed in formalin, and embedded in paraffin as previously described [75]. Sections (4 μM) were cut then rehydrated in a sequential ethanol-to-water bath and stained with hematoxylin and eosin or antigen-specific primary and secondary antibodies. Primary antibodies for immunofluorescence included: anti-GFP, anti-Bmi1, and anti-GRP78 (Abcam; Cambridge, M A); anti-phospho VASP Ser239 (Sigma; Billerica, Mass.); and anti-GUCY2C (prepared and validated in-house) [119]. Secondary antibodies were from Life Technologies (Waltham, Mass.) and specific to the primary hosts. Tyramide signal amplification [120] was used to detect GUCY2C; secondary antibodies conjugated to horseradish peroxidase were from Jackson immunoresearch Laboratories (cat #115-035-206 and #111-036-046, 1:1000 dilution), and fluorescein-conjugated tyramine was prepared from tyramine HCl (cat #T2879, Sigma) and NHS-fluorescein (cat #46410, Thermo Scientific) as described [121]. For visualization of Rosa-LacZ lineage tracing, tamoxifen-induced recombinant Cre intestines were prepared as described previously [122]. At least 4 intestinal circumference sections were evaluated per mouse.

### Crypt Isolation and Culture

Crypt isolation for subsequent analyses (enteroid assay, florescence-activated cell sorting (FACS), immunoblot) was performed using a variation of the chelation dissociation method [123]. Briefly, intestines were harvested, villi were gently scraped off for the small intestine, and tissues were minced and incubated in a 10 mM EDTA/Ca-free, Mg-free Hank's Balanced Salt Solution (HBSS) on ice for a total of 40 min, Throughout this time, solutions were intermittently shaken by hand at the speed of two shakes/second, supernatant was disposed a total of six times, and fresh EDTA/HBSS was added after each disposal. Tissue was incubated undisturbed for 30 min on ice followed by vigorous pipetting with a 10 mL pipet to dissociate the remaining crypts. Crypts were filtered through a 70 μM strainer and pelleted. For enteroid culture, the same number of crypts for each genotype (ranging from 300-1500 crypts/well) were resuspended in a matrigel droplet (BD, 354230), pipetted briefly with a 1000 μL micropipette, plated in 30 μL, and overlaid with 350 μL of intesticult media (Stem Cell Technologies, Vancouver. Canada: 06005). For FACS, crypts were incubated in 0.25% trypsin (Thermo Scientific, Philadelphia, Pa.; 15050065) at 37° C. until a single cell suspension was obtained (not more than 10 min). Cells were then filtered a second time using a 40 μM strainer and kept in EDTA solution for sorting.

### Fluorescence-Activated Cell Sorting

Cell populations from Lgr5-EGFP-CreERT2 mice were collected using a Coulter MoFlo Cell Sorter or analyzed using a BD LSRIL Live cells, determined by forward scatter, side scatter, and propidium iodide (PI, Roche), were gated negatively on CD45 (BD Pharmingen, San Jose, Calif.), then positively gated on CD241^{Low} (BD Pharmingen) [124, 125]. Finally, cells were gated negatively (for differentiated cells) and positively (for Lgr5⁺ cells) gated on endogenous eGFP fluorescence.

### Quantitative Reverse Transcriptase-Polymerase Chain Reaction (RT-qPCR)

RNA from sorted cells was amplified and reverse transcribed in situ using total RNA from the CD45⁻/CD24^{Low}/EGFP⁺ population. RNA was amplified using MessageBOOSTER cDNA Synthesis Kit for qPCR (Epicentre, Madison, Wis.) and then subjected to one-step reverse transcription polymerase chain reaction using TaqMan EZ reverse-transcription polymerase chain reaction Core Reagents and appropriate primer/probes for TaqMan GeneExpression Assays in an ABI 7900 (Applied Biosystems, Norwalk, Conn.).

### Immunoblot

Protein was extracted as described [107], quantitated using BCA assay (Pierce) and subjected to immunoblot analysis using anti-Bmi1 (Abcam; Cambridge, Mass.), anti-CHOP, anti-calreticulin, anti-phospho-EIF2α, anti-β-tubulin (Cell Signaling, Danvers, Mass.) and anti-Grp78 (Abcam). Secondary antibodies were from Santa Cruz Biotechnology (Dallas, Tex.). Molecular weight markers (Cat. #10748010, 5 μL per run, or Cat. #LC5800, 10 μL per run) for immunoblot analyses were from Invitrogen (Grand Island, N.Y.).

### Transmission Electron Microscopy

Pieces (3 cm) of intestinal tissue were placed in fixative containing 2.5% glutaraldehyde, 0.1% tannic acid, and 0.1 mol/L phosphate buffer for 5 min three times and stored at 4° C. Tissues were mounted in plastic blocks, processed through 0.1 mol/L phosphate buffer supplied with 2% OsO4 (Osmium), uranyl acetate, then dehydrated through a graded acetone sequence. After being embedded in Spurrs media, blocks were sectioned and visualized using a FEI Tecnai 12 microscope and images will be captured with an AMT digital camera. Representative electron micrographs of each group were taken (kindly performed by Timothy Schneider, Department of Pathology, Thomas Jefferson University). Cells from at least 30 crypts were enumerated per mouse.

### Statistical Analyses

All analyses were conducted in a blinded fashion. Two-tailed student's t-tests were used for single comparisons, and two-way analysis of variance (ANOVA) for multiple comparisons, unless otherwise indicated. Cohort sizes were calculated to be sufficient to detect two-tailed statistically significant differences with 95% confidence and 80% power, assuming unequal variances and allowing for unequal sample sizes between groups. P<0.05 was considered significant. Statistical analyses were performed with GraphPad Prism 6 software. Data represent mean±SEM.

Abbreviations: CBC, crypt base columnar; cGMP, cyclic GMP; ER, endoplasmic reticulum; GUCY2C, guanylyl cyclase C; ISC, intestinal stem cells; ST, bacterial heat-stable enterotoxin

### REFERENCES (WHICH ARE HEREBY INCORPORATED HEREIN BY REFERENCE)

56. Umar S. Intestinal stem cells. Curr Gastroenterol Rep. 2010; 12(5):340-348.
57. Barker N. Adult intestinal stem cells: critical drivers of epithelial homeostasis and regeneration. Nature reviews Molecular cell biology. 2014; 15(1):19-33.
58. Westphalen C B, Asfaha S, Hayakawa Y, Takemoto Y, Lukin D J, Nuber A H, Brandtner A, Setlik W, Remotti H, Muley A, Chen X, May R, Houchen C W, Fox J G, Gershon M D, Quante M, et al. Long-lived intestinal tuft cells serve as colon cancer-initiating cells. The Journal of clinical investigation. 2014; 124(3):1283-1295.
59. Luo K and Cao S S. Endoplasmic reticulum stress in intestinal epithelial cell function and inflammatory bowel disease. Gastroenterol Res Pract. 2015; 2015:328791.
60. Metcalfe C, Kljavin N M, Ybarra R and de Sauvage F J. Lgr5+ stem cells are indispensable for radiation-induced intestinal regeneration. Cell stem cell. 2014; 14(2):149-159.
61. Yan K S, Chia L A, Li X, Ootani A, Su J, Lee J Y, Su N, Luo Y, Heilshom S C, Amieva M R. Sangiorgi E, Capecchi M R and Kuo C J. The intestinal stem cell markers Bmi1 and Lgr5 identify two functionally distinct populations. Proceedings of the National Academy of Sciences of the United States of America. 2012; 109(2):466-471.
62. Davidson L A, Goldsby J S, Callaway E S, Shah M S, Barker N and Chapkin R S. Alteration of colonic stem cell gene signatures during the regenerative response to injury. Biochimica et biophysica acta. 2012; 1822(10):1600-1607.
63. Tan D W and Barker N. Intestinal stem cells and their defining niche. Current topics in developmental biology. 2014; 107:77-107.
64. Sangiorgi E and Capecchi M R. Bmi1 is expressed in vivo in intestinal stem cells. Nature genetics. 2008; 40(7):915-920.
65. Tian H, Biehs B, Warming S, Leong K G, Rangell L, Klein O D and de Sauvage F J. A reserve stem cell population in small intestine renders Lgr5-positive cells dispensable. Nature. 2011; 478(7368):255-259.
66. Takeda N, Jain R, LeBoeuf M R, Wang Q, Lu M M and Epstein J A. Interconversion between intestinal stem cell populations in distinct niches. Science. 2011: 334(6061):1420-1424.
67. Kuhn M. Molecular physiology of membrane guanylyl cyclase receptors. Physiol Rev. 2016; 96(2):751-804.
68. Pitari G M, Di Guglielmo M D, Park J, Schulz S and Waldman S A. Guanylyl cyclase C agonists regulate progression through the cell cycle of human colon carcinoma cells. Proceedings of the National Academy of Sciences of the United States of America. 2001; 98(14):7846-7851.
69. Li P, Schulz S Bombonati A, Palazzo J P, Hyslop T M, Xu Y, Baran A A, Siracusa L D, Pitari G M and Waldman S A. Guanylyl cyclase C suppresses intestinal tumorigenesis by restricting proliferation and maintaining genomic integrity. Gastroenterology. 2007; 133(2):599-607.
70. Li P, Lin J E, Chervoneva I, Schulz S, Waldman S A and Pitari G M. Homeostatic control of the crypt-villus axis by the bacterial enterotoxin receptor guanylyl cyclase C restricts the proliferating compartment in intestine. The American journal of pathology. 2007; 171(6):1847-1858.
71. Marshman E, Booth C and Potten C S. The intestinal epithelial stem cell. Bioessays. 2002; 24(1):91-98.
72. Biswas S, Davis H, Irshad S, Sandberg T, Worthley D and Leedham S. Microenvironmental control of stem cell fate in intestinal homeostasis and disease. J Pathol. 2015; 237(2):135-145.
73. Han X, Mann E, Gilbert S, Guan Y, Steinbrecher K A, Montrose M H and Cohen M B. Loss of guanylyl cyclase C (GCC) signaling leads to dysfunctional intestinal barrier. PloS one. 2011; 6(1):e16139.
74. Kuhn M. Molecular physiology of membrane guanylyl cyclase receptors. Physiological Reviews. 2016; 96(2):751-804.
75. Lin J E, Li P, Snook A E, Schulz S, Dasgupta A, Hyslop T M, Gibbons A V, Marszlowicz G, Pitari G M and Waldman S A. The hormone receptor GUCY2C suppresses intestinal tumor formation by inhibiting AKT signaling. Gastroenterology. 2010; 138(1):241-254.
76. Lin J E, Snook A E, Li P, Stoecker B A, Kim G W, Magee M S, Garcia A V, Valentino M A, Hyslop T, Schulz S and Waldman S A. GUCY2C opposes systemic genotoxic tumorigenesis by regulating AKT-dependent intestinal barrier integrity. PLoS One. 2012; 7(2):e31686.
77. Leushacke M and Barker N. Ex vivo culture of the intestinal epithelium: strategies and applications. Gut. 2014; 63(8):1345-1354.
78. Barker N, van Es J H, Kuipers J, Kujala P, van den Bom M, Cozijnsen M, Haegebarth A, Korving J, Begthel H, Peters P J and Clevers H. Identification of stem cells in small intestine and colon by marker gene Lgr5. Nature. 2007; 449(7165):1003-1007.
79. Punyashthiti K and Finkelstein R A. Enteropathogenicity of Escherichia coli. 1. Evaluation of mouse intestinal loops. Infect Immun. 1971: 4(4):473478.
80. Pitari G M. Pharmacology and clinical potential of guanylyl cyclase C agonists in the treatment of ulcerative colitis. Drug design, development and therapy. 2013; 7:351-360.
81. Poppe H, Rybalkin S D, Rehmann H, Hinds T R, Tang X B, Christensen A E, Schwede F, Genieser H G, Bos J L, Doskeland S O, Beavo J A and Butt E. Cyclic nucleotide analogs as probes of signaling pathways. Nat Methods. 2008; 5(4):277-278.
82. Heijmans J, van Lidth de Jeude J F, Koo B K, Rosekrans S L, Wielenga M C, van de Wetering M, Ferrante M, Lee A S, Onderwater J J, Paton J C, Paton A W, Mommaas A M, Kodach L L, Hardwick J C, Hommes D W, Clevers H, et al. E R stress causes rapid loss of intestinal epithelial sternness through activation of the unfolded protein response. Cell reports. 2013; 3(4):1128-1139.
83. Niederreiter L, Fritz T M, Adolph T E, Krismer A M, Offner F A, Tschurtschenthaler M. Flak M B, Hosomi S. Tomczak M F, Kaneider N C, Sarcevic E, Kempster S L, Raine T, Esser D, Rosenstiel P, Kohno K, et al. E R stress transcription factor Xbp suppresses intestinal tumorigenesis and directs intestinal stem cells. The Journal of experimental medicine. 2013; 210(10):2041-2056.
84. Pitari G M, Zingman L V, Hodgson D M, Alekseev A E, Kazerounian S, Bienengraeber M, Hajnoczky G, Terzic A and Waldman S A. Bacterial enterotoxins are associated with resistance to colon cancer. Proceedings of the National Academy of Sciences of the United States of America. 2003; 100(5):2695-2699.
85. Lee A S. The E R chaperone and signaling regulator GRP78/BiP as a monitor of endoplasmic reticulum stress. Methods. 2005; 35(4):373-381.
86. Xu C, Bailly-Maitre B and Reed J C. Endoplasmic reticulum stress: cell life and death decisions. The Journal of clinical investigation. 2005; 115(10):2656-2664.
87. Urra H, Dufey E, Lisbona F, Rojas-Rivera D and Hetz C. When ER stress reaches a dead end. Biochimica et biophysica acta. 2013; 1833(12):3507-3517.
88. Rutkowski D T, Arnold S M, Miller C N, Wu J, Li J, Gunnison K M, Mori K, Sadighi Akha A A, Raden D and Kaufman R J. Adaptation to E R stress is mediated by differential stabilities of pro-survival and pro-apoptotic mRNAs and proteins. PLoS Biol. 2006; 4(11):e374.
89. Tsang K Y, Chan D, Bateman J F and Cheah K S. In vivo cellular adaptation to ER stress: survival strategies with double-edged consequences. J Cell Sci. 2010; 123(Pt 13):2145-2154.
90. Cortez L and Sim V. The therapeutic potential of chemical chaperones in protein folding diseases. Prion. 2014; 8(2).
91. Bach S P, Renehan A G and Potten C S. Stem cells: the intestinal stem cell as a paradigm. Carcinogenesis. 2000; 21(3):469-476.
92. Withers H R and Elkind M M. Micmcolony survival assay for cells of mouse intestinal mucosa exposed to radiation. Int J Radiat Biol Relat Stud Phys Chem Med. 1970; 17(3):261-267.
93. Li N, Yousefi M, Nakauka-Ddamba A, Jain R, Tobias J, Epstein J A, Jensen S T and Lengner C J. Single-cell analysis of proxy reporter allele-marked epithelial cells establishes intestinal stem cell hierarchy. Stem Cell Reports. 2014; 3(5):876-891.
94. Kim E, Davidson L A, Zoh R S, Hensel M E, Patil B S, Jayaprakasha G K, Callaway E S, Allred C D, Turner N D, Weeks B R and Chapkin R S. Homeostatic responses of colonic LGR5+ stem cells following acute in vivo exposure to a genotoxic carcinogen. Carcinogenesis. 2016; 37(2):206-214.
95. Mills J C and Sansom O J. Reserve stem cells: Differentiated cells reprogram to fuel repair, metaplasia, and neoplasia in the adult gastrointestinal tract. Sci Signal. 2015; 8(385):re8.
96. itzkovitz S, Lyubimova A, Blat I C, Maynard M, van Es J, Lees J, Jacks T, Clevers H and van Oudenaarden A. Single-molecule transcript counting of stem-cell markers in the mouse intestine. Nat Cell Biol. 2012; 14(1):106-114.
97. Tetteh P W, Farin H F and Clevers H. Plasticity within stem cell hierarchies in mammalian epithelia. Trends Cell Biol. 2015; 25(2):100-108.
98. Donati G and Watt F M. Stem cell heterogeneity and plasticity in epithelia. Cell stem cell. 2015; 16(5):465-476.
99. Qiu W, Wang X. Buchanan M, He K, Sharma R, Zhang L, Wang Q and Yu J. ADAR1 is essential for intestinal homeostasis and stem cell maintenance. Cell Death Dis. 2013; 4:e599.
100. Brenna O, Fumes M W, Munkvold B, Kidd M, Sandvik A K and Gustafsson B I. Cellular localization of guanylin and uroguanylin mRNAs in human and rat duodenal and colonic mucosa. Cell Tissue Res. 2016.
101. Garin-Laflam M P, Steinbrecher K A, Rudolph J A, Mao J and Cohen M B. Activation of guanylate cyclase C signaling pathway protects intestinal epithelial cells from acute radiation-induced apoptosis. Am J Physiol Gastrointest Liver Physiol. 2009; 296(4):G740-749.
102. Harmel-Laws E, Mann E A, Cohen M B and Steinbrecher K A. Guanylate cyclase C deficiency causes severe inflammation in a murine model of spontaneous colitis. PloS one. 2013; 8(11):e79180.
103. Steinbrecher K A, Harmel-Laws E, Garin-Laflam M P, Mann E A, Bezerra L D, Hogan S P and Cohen M B. Murine guanylate cyclase C regulates colonic injury and inflammation. J Immunol. 2011; 186(12):7205-7214.
104. Notterman D A, Alon U, Sierk A J and Levine A J. Transcriptional gene expression profiles of colorectal adenoma, adenocarcinoma, and normal tissue examined by oligonucleotide arrays. Cancer Res. 2001; 61(7):3124-3130.
105. Steinbrecher K A, Tuohy T M, Heppner Goss K, Scott M C, Witte D P, Groden J and Cohen M B. Expression of guanylin is downregulated in mouse and human intestinal adenomas. Biochem Biophys Res Commun. 2000; 273(1):225-230.
106. Wilson C, Lin J E, Li P, Snook A E, Gong J, Sato T, Liu C, Girondo M A, Rui H, Hyslop T and Waldman S A. The paracrine hormone for the GUCY2C tumor suppressor, guanylin, is universally lost in colorectal cancer. Cancer Epidemiol Biomarkers Prev. 2014 23(11):2328-2337.
107. Lin J E, Colon-Gonzalez F, Blomain E, Kim G W, Aing A, Stoecker B, Rock J, Snook A E, Zhan T, Hyslop T M, Tomczak M, Blumberg R S and Waldman S A. Obesity-induced colorectal cancer Is driven by caloric silencing of the guanylin-GUCY2C paracrine signaling axis. Cancer Res. 2016; 76(2):339-346.
108. Barker N, Ridgway R A, van Es J H, van de Wetering M, Begthel H, van den Born M, Danenberg E, Clarke A R, Sansom O J and Clevers H. Crypt stem cells as the cells-of-origin of intestinal cancer. Nature. 2009; 457(7229):608-611.
109. Proctor E, Waghray M, Lee C J, Heidt D G, Yalamanchili M, Li C, Bednar F and Simeone D M. Bmi1 enhances tumorigenicity and cancer stem cell function in pancreatic adenocarcinoma. PloS one. 2013; 8(2):eSS820.
110. Zhu D, Wan X, Huang H, Chen X, Liang W, Zhao F, Lin T, Han J and Xie W. Knockdown of Bmi1 inhibits the stemness properties and tumorigenicity of human bladder cancer stem cell-like side population cells. Oncol Rep. 2014; 31(2):727-736.
111. Brenna O, Bruland T, Fumes M W, Granlund A, Drozdov 1, Emgard J, Bronstad G. Kidd M, Sandvik A K and Gustafsson B I. The guanylate cyclase-C signaling pathway is down-regulated in inflammatory bowel disease. Scand J Gastroenterol. 2015; 50(10):1241-1252.
112. Camilleri M. Guanylate cyclase C agonists: emerging gastrointestinal therapies and actions. Gastroenterology. 2015; 148(3):483-487.
113. Shailubhai K, Yu H H, Karunanandaa K, Wang J Y, Eber S L, Wang Y, Joo N S, Kim H D, Miedema B W, Abbas S Z, Boddupalli S S, Currie M G and Forte L R. Uroguanylin treatment suppresses polyp formation in the Apc(Min/+) mouse and induces apoptosis in human colon adenocarcinoma cells via cyclic GMP. Cancer Res. 2000; 60(18):5151-5157.
114. Weinberg D S, Lin J E, Foster N R, Della'Zanna G, Umar A, Seisler D, Kraft W K, Kastenberg D M, Katz L C, Limburg P J and Waldman S A. Bioactivity of oral linaclotide in human colorectum for cancer chemoprevention. Cancer Prev Res (Phila). 2017; 10(6):345-354.
115. Shailubhai K, Palejwala V, Arjunan K P, Saykhedkar S, Nefsky B, Foss J A, Comiskey S, Jacob G S and Plevy S E. Plecanatide and dolcanatide, novel guanylate cyclase-C agonists, ameliorate gastrointestinal inflammation in experimental models of murine colitis. World J Gastrointest Pharmacol Ther. 2015; 6(4):213-222.
116. Kantara C, Moya S M, Houchen C W, Umar S, Ullrich R L, Singh P and Camey D H. Novel regenerative peptide TP508 mitigates radiation-induced gastrointestinal damage by activating stem cells and preserving crypt integrity. Lab Invest. 2015; 95(11):1222-1233.
117. Booth C, Tudor G, Tudor J, Katz B P and MacVittie T J. Acute gastrointestinal syndrome in high-dose irradiated mice. Health Phys. 2012; 103(4):383-399.
118. Schulz S, Lopez M J, Kuhn M and Garbers D L. Disruption of the guanylyl cyclase-C gene leads to a paradoxical phenotype of viable but heat-stable enterotoxin-resistant mice. The Journal of clinical investigation. 1997; 100(6):1590-1595.
119. Marszalowicz G P, Snook A E, Magee M S, Merlino D, Berman-Booty L D and Waldman S A. GUCY2C lysosomotropic endocytosis delivers immunotoxin therapy to metastatic colorectal cancer. Oncotarget. 2014; 5(19):9460-9471.
120. Faget L and Hnasko T S. Tyramide Signal Amplification for Immunofluorescent Enhancement. Methods in molecular biology (Clifton, N.J.). 2015; 1318:161-172.
121. Hopman A H, Ramaekers F C and Speel E J. Rapid synthesis of biotin-, digoxigenin-, trinitrophenyl-, and fluorochrome-labeled tyramides and their application for In situ hybridization using CARD amplification. The journal of histochemistry and cytochemistry official journal of the Histochemistry Society. 1998; 46(6):771-777.
122. el Marjou F, Janssen K P, Chang B H, Li M, Hindie V, Chan L, Louvard D, Chambon P, Metzger D and Robine S. Tissue-specific and inducible Cre-mediated recombination in the gut epithelium. Genesis. 2004; 39(3):186-193.
123. Yilmaz O H, Katajisto P, Lamming D W, Gultekin Y, Bauer-Rowe K E, Sengupta S, Birsoy K, Dursun A, Yilmaz V O, Selig M, Nielsen G P, Mino-Kenudson M, Zukerberg L R, Bhan A K, Deshpande V and Sabatini D M. mTORC1 in the Paneth cell niche couples intestinal stem-cell function to calorie intake. Nature. 2012; 486(7404):490-495.
124. von Furstenberg R J, Gulati A S, Baxi A, Doherty J M, Stappenbeck T S, Gracz A D, Magness S T and Henning S J. Sorting mouse jejunal epithelial cells with CD24 yields a population with characteristics of intestinal stem cells. Am J Physiol Gastrointest Liver Physiol. 2011; 300(3):G409-417.
125. Sato T, van Es J H, Snippert H J, Stange D E, Vries R G, van den Born M, Barker N, Shroyer N F, van de Wetering M and Clevers H. Paneth cells constitute the niche for Lgr5 stem cells in intestinal crypts. Nature. 2011; 469(7330):415-418.

1. A method of treating an individual who has cancer in an individual who has been identified as having cancer which lacks functional guanylyl cyclase C, the method comprising:
administering to gastrointestinal cells in the individual who has been identified as having cancer which lacks functional guanylyl cyclase C, an amount of one or more guanylyl cyclase C agonist compounds sufficient to activate guanylyl cyclase C of the gastrointestinal cells and elevate intracellular cGMP in the gastrointestinal cells to a level that protects gastrointestinal cells from genotoxic damage by causing
arrest of cell proliferation of the gastrointestinal cells, and/or
inhibition of DNA synthesis and prolongation of cell cycle of the gastrointestinal cells by imposing a G1-S delay and/or
genomic integrity of the gastrointestinal cells to be maintained by enhanced DNA damage sensing and repair; and
administering chemotherapy and/or radiation therapy to kill cancer cells that lack functional guanylyl cyclase C,
wherein the chemotherapy and/or radiation is administered when normal gastrointestinal cells are protected from genotoxic damage cell by the effects of elevated intracellular cGMP in the gastrointestinal cells.

2. The method of claim 1 wherein the cancer which lacks functional guanylyl cyclase C is selected from the group consisting of: colorectal cancer which lacks functional guanylyl cyclase C, esophageal cancer which lacks functional guanylyl cyclase C, pancreatic cancer which lacks functional guanylyl cyclase C, liver cancer which lacks functional guanylyl cyclase C, stomach cancer which lacks functional guanylyl cyclase C, biliary system cancer which lacks functional guanylyl cyclase C, cancer of the peritoneum which lacks functional guanylyl cyclase C, bladder cancer which lacks functional guanylyl cyclase C, kidney cancer which lacks functional guanylyl cyclase C, cancer of the ureter which lacks functional guanylyl cyclase C, prostate cancer which lacks functional guanylyl cyclase C, ovarian cancer which lacks functional guanylyl cyclase C, uterus cancer which lacks functional guanylyl cyclase C and soft tissues of the abdomen and pelvis such as sarcomas which lack functional guanylyl cyclase C.

3. The method of claim 1 further comprising identifying the cancer as lacking functional p53 and administering one or more active agents selected from the group consisting of: Guanylyl cyclase A (GCA) agonists (ANP, BNP), Guanylyl cyclase B (GCB) agonists (CNP), Soluble guanylyl cyclase activators (nitric oxide, nitrovasodilators, protoprophyrin IX, and direct activators), PDE Inhibitors, MRP inhibitors, cyclic GMP and cGMP analogues and optionally.

4. The method of claim 1 further comprising identifying the cancer as lacking functional p53 and administering one or more active agents selected from the group consisting of: Guanylyl cyclase A (GCA) agonists (ANP, BNP), Guanylyl cyclase B (GCB) agonists (CNP), Soluble guanylyl cyclase activators (nitric oxide, nitrovasodilators, protoprophyrin IX, and direct activators), PDE Inhibitors, MRP inhibitors, cyclic GMP and cGMP analogues, wherein the cancer is identified as lacking functional p53 by detecting the absence of p53 or RNA that encodes p53 in a sample of cancer cells from the individual.

5. The method of claim 1, comprising:
identifying the individual as having cancer which lacks functional guanylyl cyclase C.

6. The method of claim 5 comprising the step of identifying the individual as having cancer which lacks functional guanylyl cyclase C by detecting the absence of guanylyl cyclase C or RNA that encodes guanylyl cyclase C in a sample of cancer cells from the individual.

7. The method of claim 5 comprising the step of identifying the individual as having cancer which lacks functional guanylyl cyclase C by detecting the absence of guanylyl cyclase C in a sample of cancer cells from the individual by contacting the sample of cancer cells with a reagent that binds to guanylyl cyclase C and detecting the absence of binding of the reagent to the sample cancer cells.

8. The method of claim 5 comprising the step of identifying the individual as having cancer which lacks functional guanylyl cyclase C by detecting the absence of guanylyl cyclase C in a sample of cancer cells from the individual by contacting the sample of cancer cells with a reagent that binds to guanylyl cyclase C and detecting the absence of binding of the reagent to the sample cancer cells, wherein the reagent is an anti-guanylyl cyclase C or a guanylyl cyclase C ligand.

9. The method of claim 5 the individual as having cancer which lacks functional guanylyl cyclase C by detecting the absence of RNA that encodes guanylyl cyclase C in a sample of cancer cells from the individual by performing PCR on mRNA from the sample of cancer cells using PCR primers that amplify RNA that encodes guanylyl cyclase C and detecting the absence of amplified RNA in the sample cancer cells or by contacting an oligonucleotide with mRNA from the sample of cancer cells wherein the oligonucleotide has a sequence that hybridizes to RNA that encodes guanylyl cyclase C and detecting the absence of oligonucleotide hybridized to mRNA from the sample of cancer cells.

10-12. (canceled)

13. A method of treating an individual who has primary colorectal cancer in an individual who has been identified as having primary colorectal cancer which lacks functional p53, the method comprising:
administering to gastrointestinal cells in the individual who has been identified as having primary colorectal cancer which lacks functional p53, an amount of the one or more guanylyl cyclase C agonist compounds sufficient to activate guanylyl cyclase C of the gastrointestinal cells and elevate intracellular cGMP in the gastrointestinal cells to a level that protects gastrointestinal cells from genotoxic damage by causing
arrest of cell proliferation of the gastrointestinal cells, and/or
inhibition of DNA synthesis and prolongation of cell cycle of the gastrointestinal cells by imposing a G1-S delay and/or
genomic integrity of the gastrointestinal cells to be maintained by enhanced DNA damage sensing and repair; and
administering chemotherapy and/or radiation therapy to kill primary colorectal cancer cells that lack functional p53,
wherein the chemotherapy and/or radiation is administered when normal gastrointestinal cells are protected from genotoxic damage cell by the effects of elevated intracellular cGMP in the gastrointestinal cells.

14. The method of claim 13, further comprising:
identifying the individual as having primary colorectal cancer which lacks functional p53.

15. A method of treating an individual who has cancer, the method comprising:
administering to intestinal stem cells in the individual an amount of one or more guanylyl cyclase C agonist compounds sufficient to activate guanylyl cyclase C of the intestinal stem cells and elevate intracellular cGMP in the intestinal stem cells to a level that that causes an increase in intestinal stem cell number and a shift of relative balance of intestinal stem cells to increase intestinal stem cells with a Lgr5+ active phenotype and to decrease intestinal stem cells with a Bmi1+ reserve phenotype,
administering chemotherapy and/or radiation therapy to kill cancer cells when intestinal stem cell number is increased and relative balance of intestinal stem cells is shifted to increase intestinal stem cells with a Lgr5+ active phenotype and to decrease intestinal stem cells with a Bmi1+ reserve phenotype,
wherein the chemotherapy and/or radiation administered when intestinal stem cell number is increased and relative balance of intestinal stem cells is shifted to increase intestinal stem cells with a Lgr5+ active phenotype and to decrease intestinal stem cells with a Bmi1+ reserve phenotype results in fewer gastrointestinal side effects.

16. The method of claim 1 wherein the individual is administered chemotherapy.

17. The method of claim 1 wherein the individual is administered radiation.

18. The method of claim 1 wherein the individual is administered abdominopelvic radiation.

19. The method of claim 1 comprising administering to said individual a GCC agonist peptide.

20. The method of claim 1 comprising administering to said individual a GCC agonist peptide selected from the group consisting of guanylin, uroguanylin, SEQ ID NOs:2, 3 and 5-60.

21. (canceled)

22. The method of claim 1 wherein the GCC agonist compound is administered by oral administration.

23. The method of claim 1 wherein the GCC agonist compound is administered by oral administration in a controlled release composition.

24. The method of claim 1 wherein GCC agonist compound is administered to said individual 24 hours prior to administering to said individual chemotherapy or radiation an amount sufficient to treat cancer 48 hours prior to administering to said individual chemotherapy or radiation an amount sufficient to treat cancer 72 hours prior to administering to said individual chemotherapy or radiation an amount sufficient to treat cancer; or 96 hours prior to administering to said individual chemotherapy or radiation an amount sufficient to treat cancer.

25. The method of claim 1 wherein the individual is administered a guanylyl cyclase C agonist daily for 2, 3, 4, 5, 6, 7, 8, 9, 10, 11, 12, 13 or 14 days.

26. The method of claim 1 wherein the GCC agonist compound is administered in multiple doses.

27. The method of claim 1 wherein tumor is surgically removed from the individual prior to administration of the guanylyl cyclase C agonist.

28. The method of claim 1 wherein the individual is identified as responding to protective action of guanylyl cyclase C agonist compound by detecting changes in bowel movements of the individual following administration of the guanylyl cyclase C agonist, wherein treatment proceeds upon detection changes in bowel movements of the individual following administration of the guanylyl cyclase C agonist.

29. A method of treating an individual who has been identified as having cancer which lacks functional p53, the method comprising:
identifying the individual as having cancer which lacks functional p53;
administering to gastrointestinal cells in the individual an amount of one or more compounds selected from the group consisting of: Guanylyl cyclase A (GCA) agonists (ANP, BNP), Guanylyl cyclase B (GCB) agonists (CNP), Soluble guanylyl cyclase activators (nitric oxide, nitrovasodilators, protoprophyrin IX, and direct activators), PDE Inhibitors, MRP inhibitors, cyclic GMP and cGMP analogues in an amount sufficient to elevate intracellular cGMP in normal cells and protect the normal cells from genotoxic effects of chemotherapy and/or radiation; and
administering chemotherapy and/or radiation therapy to kill cancer cells, wherein the chemotherapy and/or radiation is administered when the normal cells are protected from genotoxic effects of chemotherapy and/or radiation.

30. The method of claim 29 comprising the step of identifying the individual as having cancer which lacks functional p53 by detecting the absence of p53 or RNA that encodes p53 in a sample of cancer cells from the individual.

31-36. (canceled)
